SEQUENCE LISTING (96 sequences)

SEQ ID NO: 1 - protein, 746 aa, Mus musculus
MGQTGKKSEKGPVCWRKRVKSEYMRLRQLKRFRRADEVKTMFSSNRQKILERTETLNQEWKQRRIQPVHIMTSVSSLRGTRECSVTSDLDFPAQVIPLKTLNAVASVPIMYSWSPLQQNFMVEDETVLHNIPYMGDEVLDQDGTFIEELIKNYDGKVHGDRECGFINDEIFVELVNALGQYNDDDDDDDGDDPDEREEKQKDLEDNRDDKETCPPRKFPADKIFEAISSMFPDKGTAEELKEKYKELTEQQLPGALPPECTPNIDGPNAKSVQREQSLHSFHTLFCRRCFKYDCFLHPFHATPNTYKRKNTETALDNKPCGPQCYQHLEGAKEFAAALTAERIKTPPKRPGGRRRGRLPNNSSRPSTPTISVLESKDTDSDREAGTETGGENNDKEEEEKKDETSSSSEANSRCQTPIKMKPNIEPPENVEWSGAEASMFRVLIGTYYDNFCAIARLIGTKTCRQVYEFRVKESSIIAPVPTEDVDTPPRKKKRKHRLWAAHCRKIQLKKDGSSNHVYNYQPCDHPRQPCDSSCPCVIAQNFCEKFCQCSSECQNRFPGCRCKAQCNTKQCPCYLAVRECDPDLCLTCGAADHWDSKNVSCKNCSIQRGSKKHLLLAPSDVAGWGIFIKDPVQKNEFISEYCGEIISQDEADRRGKVYDKYMCSFLFNLNNDFVVDATRKGNKIRFANHSVNPNCYAKVMMVNGDHRIGIFAKRAIQTGEELFFDYRYSQADALKYVGIEREMEIP

SEQ ID NO: 2 - protein, 265 aa, Mus musculus
TEDVDTPPRKKKRKHRLWAAHCRKIQLKKDGSSNHVYNYQPCDHPRQPCDSSCPCVIAQNFCEKFCQCSSECQNRFPGCRCKAQCNTKQCPCYLAVRECDPDLCLTCGAADHWDSKNVSCKNCSIQRGSKKHLLLAPSDVAGWGIFIKDPVQKNEFISEYCGEIISQDEADRRGKVYDKYMCSFLFNLNNDFVVDATRKGNKIRFANHSVNPNCYAKVMMVNGDHRIGIFAKRAIQTGEELFFDYRYSQADALKYVGIEREMEIP

SEQ ID NO: 3 - protein, 45 aa, Homo sapiens
MSRRKQSNPRQIKRSLGDMEAREEVQLVGASHMEQKATAPEAPSP

SEQ ID NO: 4 - protein, 337 aa, Homo sapiens
PRQNLKCVRILKQFHKDLERELLRRHHRSKTPRHLDPSLANYLVQKAKQRRALRRWEQELNAKRSHLGRITVENEVDLDGPPRAFVYINEYRVGEGITLNQVAVGCECQDCLWAPTGGCCPGASLHKFAYNDQGQVRLRAGLPIYECNSRCRCGYDCPNRVVQKGIRYDLCIFRTDDGRGWGVRTLEKIRKNSFVMEYVGEIITSEEAERRGQIYDRQGATYLFDLDYVEDVYTVDAAYYGNISHFVNHSCDPNLQVYNVFIDNLDERLPRIAFFATRTIRAGEELTFDYNMQVDPVDMESTRMDSNFGLAGLPGSPKKRVRIECKCGTESCRKYLF

SEQ ID NO: 5 - protein, 381 aa, Homo sapiens
GSAAIAEVLLNARCDLHAVNYHGDTPLHIAARESYHDCVLLFLSRGANPELRNKEGDTAWDLTPERSDVWFALQLNRKLRLGVGNRAIRTEKIICRDVARGYENVPIPCVNGVDGEPCPEDYKYISENCETSTMNIDRNITHLQHCTCVDDCSSSNCLCGQLSIRCWYDKDGRLLQEFNKIEPPLIFECNQACSCWRNCKNRVVQSGIKVRLQLYRTAKMGWGVRALQTIPQGTFICEYVGELISDAEADVREDDSYLFDLDNKDGEVYCIDARYYGNISRFINHLCDPNIIPVRVFMLHQDLRFPRIAFFSSRDIRTGEELGFDYGDRFWDIKSKYFTCQCGSEKCKHSAEAIALEQSRLARLDPHPELLPELGSLPPVN

SEQ ID NO: 6 - protein, 313 aa, Homo sapiens
RAPSRLQMFFANNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACV

SEQ ID NO: 7 - protein, 74 aa, Homo sapiens
TLVTFKDVFVDFTREEWKLLDTAQQIVYRNVMLENYKNLVSLGYQLTKPDVILRLEKGEEPWLVEREIHQETHP

SEQ ID NO: 8 - protein, 1508 aa, Artificial Sequence
  Description of Artificial Sequence Synthetic polypeptide
MPKKKRKVGGSGGSDYKDHDGDYKDHDIDYKDDDDKGGGSGGGSGTGGSGGSGGSGGSGGSGRPMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQKKKRKVGLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGSGGSGGSGGSGGSASGGGSGGGSKRPAATKKAGQAKKKKGGSGSGATNFSLLKQAGDVEENPGPAAA

SEQ ID NO: 9 - protein, 2256 aa, Artificial Sequence
  Description of Artificial Sequence Synthetic polypeptide
  MOD_RES (47)..(792): This region may encompass one of the following sequences “MGQTGKKSEKGPVCWRKRVKSEYMRLRQLKRFRRADEVKTMFSSNRQKILERTETLN QEWKQRRIQPVHIMTSVSSLRGTRECSVTSDLDFPAQVIPLKTLNAVASVPIMYSWS PLQQNFMVEDETVLHNIPYMGDEVLDQDGTFIEELIKNYDGKVHGDRECGFINDEIF
  MOD_RES (47)..(792): CONT. FROM ABOVE VELVNALGQYNDDDDDDDGDDPDEREEK QKDLEDNRDDKETCPPRKFPADKIFEAISSMFPDKGTAEELKEKYKELTEQQLPGALPPECTPNI DGPNAKSVQREQSLHSFHTLFCRRCFKYDCFLHPFHATPNTYKRKNTETALDNKPCGPQCYQHLE GAKEFAAALTAERIKTPPKRPGGRRRGRLPNNSSRPSTPTISVLESKDTDSD
  MOD_RES (47)..(792): CONT. FROM ABOVE REAGTETGGENNDKEEEEKKDETSSSSE ANSRCQTPIKMKPNIEPPENVEWSGAEASMFRVLIGTYYDNFCAIARLIGTKTCRQVYEFRVKES SIIAPVPTEDVDTPPRKKKRKHRLWAAHCRKIQLKKDGSSNHVYNYQPCDHPRQPCDSSCPCVIA QNFCEKFCQCSSECQNRFPGCRCKAQCNTKQCPCYLAVRE
  MOD_RES (47)..(792): CONT. FROM ABOVE CDPDLCLTCGAADHWDSKNVSCKNCSIQ RGSKKHLLLAPSDVAGWGIFIKDPVQKNEFISEYCGEIISQDEADRRGKVYDKYMCSFLFNLNND FVVDATRKGNKIRFANHSVNPNCYAKVMMVNGDHRIGIFAKRAIQTGEELFFDYRYSQADALKYV GIEREMEIP” or “TEDVDTPPRKKKRKHRLWAAHCRKIQLKKDGSSNHVYN
  MOD_RES (47)..(792): CONT. FROM ABOVE YQPCDHPRQPCDSSCPCVIAQNFCEKFC QCSSECQNRFPGCRCKAQCNTKQCPCYLAVRECDPDLCLTCGAADHWDSKNVSCKNCSIQRGSKK HLLLAPSDVAGWGIFIKDPVQKNEFISEYCGEIISQDEADRRGKVYDKYMCSFLFNLNNDFVVDA TRKGNKIRFANHSVNPNCYAKVMMVNGDHRIGIFAKRAIQTGEELFFDYRY
  MOD_RES (47)..(792): CONT. FROM ABOVE SQADALKYVGIEREMEIP” or “MSRRKQSNPRQIKRSLGDMEAREEVQLVGASHMEQKATAPEAPSP” or “PRQNLKCV RILKQFHKDLERELLRRHHRSKTPRHLDPSLANYLVQKAKQRRALRRWEQELNAKRSHLG RITVENEVDLDGPPRAFVYINEYRVGEGITLNQVAVGCECQDCLWAPTGGCCPGASLHKF AYNDQGQVRL
  MOD_RES (47)..(792): CONT. FROM ABOVE RAGLPIYECNSRCRCGYDCPNRVVQKGI RYDLCIFRTDDGRGWGVRTLEKIRKNSFVMEYVGEIITSEEAERRGQIYDRQGATYLFDLDYVED VYTVDAAYYGNISHFVNHSCDPNLQVYNVFIDNLDERLPRIAFFATRTIRAGEELTFDYNMQVDP VDMESTRMDSNFGLAGLPGSPKKRVRIECKCGTESCRKYLF” or “GSAAIA
  MOD_RES (47)..(792): CONT. FROM ABOVE EVLLNARCDLHAVNYHGDTPLHIAARES YHDCVLLFLSRGANPELRNKEGDTAWDLTPERSDVWFALQLNRKLRLGVGNRAIRTEKIICRDVA RGYENVPIPCVNGVDGEPCPEDYKYISENCETSTMNIDRNITHLQHCTCVDDCSSSNCLCGQLSI RCWYDKDGRLLQEFNKIEPPLIFECNQACSCWRNCKNRVVQSGIKVRLQLY
  MOD_RES (47)..(792): CONT. FROM ABOVE RTAKMGWGVRALQTIPQGTFICEYVGEL ISDAEADVREDDSYLFDLDNKDGEVYCIDARYYGNISRFINHLCDPNIIPVRVFMLHQDLRFPRI AFFSSRDIRTGEELGFDYGDRFWDIKSKYFTCQCGSEKCKHSAEAIALEQSRLARLDPHPELLPE LGSLPPVN” or “RAPSRLQMFFANNHDQEFDPPKVYPPVPAEKRKPIRVLS
  MOD_RES (47)..(792): CONT. FROM ABOVE LFDGIATGLLVLKDLGIQVDRYIASEVC EDSITVGMVRHQGKIMYVGDVRSVTQKHIQEWGPFDLVIGGSPCNDLSIVNPARKGLYEGTGRLF FEFYRLLHDARPKEGDDRPFFWLFENVVAMGVSDKRDISRFLESNPVMIDAKEVSAAHRARYFWG NLPGMNRPLASTVNDKLELQECLEHGRIAKFSKVRTITTRSNSIKQGKDQH
  MOD_RES (47)..(792): CONT. FROM ABOVE FPVFMNEKEDILWCTEMERVFGFPVHYT DVSNMSRLARQRLLGRSWSVPVIRHLFAPLKEYFACV” or “TLVTFKDVFVDFTREEWKLLDT AQQIVYRNVMLENYKNLVSLGYQLTKPDVILRLEKGEEPWLVEREIHQETHP”
  See specification as filed for detailed description of substitutions and preferred embodiments
MPKKKRKVGGSGGSDYKDHDGDYKDHDIDYKDDDDKGGGSGGGSGTXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXSTGGSGGSGGSGGSGGSGRPMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQKKKRKVGLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGSGGSGGSGGSGGSASGGGSGGGSKRPAATKKAGQAKKKKGGSGSGATNFSLLKQAGDVEENPGPAAA

SEQ ID NO: 10 - protein, 2256 aa, Artificial Sequence
  Description of Artificial Sequence Synthetic polypeptide
  MOD_RES (1457)..(2202): This region may encompass one of the following sequences “MGQTGKKSEKGPVCWRKRVKSEYMRLRQLKRFRRADEVKTMFSSNRQKILERTETLN QEWKQRRIQPVHIMTSVSSLRGTRECSVTSDLDFPAQVIPLKTLNAVASVPIMYSWS PLQQNFMVEDETVLHNIPYMGDEVLDQDGTFIEELIKNYDGKVHGDRECGFINDEIF
  MOD_RES (1457)..(2202): CONT. FROM ABOVE VELVNALGQYNDDDDDDDGDDPDEREEK QKDLEDNRDDKETCPPRKFPADKIFEAISSMFPDKGTAEELKEKYKELTEQQLPGALPPECTPNI DGPNAKSVQREQSLHSFHTLFCRRCFKYDCFLHPFHATPNTYKRKNTETALDNKPCGPQCYQHLE GAKEFAAALTAERIKTPPKRPGGRRRGRLPNNSSRPSTPTISVLESKDTDSD
  MOD_RES (1457)..(2202): CONT. FROM ABOVE REAGTETGGENNDKEEEEKKDETSSSSE ANSRCQTPIKMKPNIEPPENVEWSGAEASMFRVLIGTYYDNFCAIARLIGTKTCRQVYEFRVKES SIIAPVPTEDVDTPPRKKKRKHRLWAAHCRKIQLKKDGSSNHVYNYQPCDHPRQPCDSSCPCVIA QNFCEKFCQCSSECQNRFPGCRCKAQCNTKQCPCYLAVRE
  MOD_RES (1457)..(2202): CONT. FROM ABOVE CDPDLCLTCGAADHWDSKNVSCKNCSIQ RGSKKHLLLAPSDVAGWGIFIKDPVQKNEFISEYCGEIISQDEADRRGKVYDKYMCSFLFNLNND FVVDATRKGNKIRFANHSVNPNCYAKVMMVNGDHRIGIFAKRAIQTGEELFFDYRYSQADALKYV GIEREMEIP” or “TEDVDTPPRKKKRKHRLWAAHCRKIQLKKDGSSNHVYN
  MOD_RES (1457)..(2202): CONT. FROM ABOVE YQPCDHPRQPCDSSCPCVIAQNFCEKFC QCSSECQNRFPGCRCKAQCNTKQCPCYLAVRECDPDLCLTCGAADHWDSKNVSCKNCSIQRGSKK HLLLAPSDVAGWGIFIKDPVQKNEFISEYCGEIISQDEADRRGKVYDKYMCSFLFNLNNDFVVDA TRKGNKIRFANHSVNPNCYAKVMMVNGDHRIGIFAKRAIQTGEELFFDYRY
  MOD_RES (1457)..(2202): CONT. FROM ABOVE SQADALKYVGIEREMEIP” or “MSRRKQSNPRQIKRSLGDMEAREEVQLVGASHMEQKATAPEAPSP” or “PRQNLKCVR ILKQFHKDLERELLRRHHRSKTPRHLDPSLANYLVQKAKQRRALRRWEQELNAKRSHLGRI TVENEVDLDGPPRAFVYINEYRVGEGITLNQVAVGCECQDCLWAPTGGCCPGASLHKFAYN DQGQVRL
  MOD_RES (1457)..(2202): CONT. FROM ABOVE RAGLPIYECNSRCRCGYDCPNRVVQKGI RYDLCIFRTDDGRGWGVRTLEKIRKNSFVMEYVGEIITSEEAERRGQIYDRQGATYLFDLDYVED VYTVDAAYYGNISHFVNHSCDPNLQVYNVFIDNLDERLPRIAFFATRTIRAGEELTFDYNMQVDP VDMESTRMDSNFGLAGLPGSPKKRVRIECKCGTESCRKYLF” or “GSAAIA
  MOD_RES (1457)..(2202): CONT. FROM ABOVE EVLLNARCDLHAVNYHGDTPLHIAARE SYHDCVLLFLSRGANPELRNKEGDTAWDLTPERSDVWFALQLNRKLRLGVGNRAIRTEKIICRD VARGYENVPIPCVNGVDGEPCPEDYKYISENCETSTMNIDRNITHLQHCTCVDDCSSSNCLCGQ LSIRCWYDKDGRLLQEFNKIEPPLIFECNQACSCWRNCKNRVVQSGIKVRLQLY
  MOD_RES (1457)..(2202): CONT. FROM ABOVE RTAKMGWGVRALQTIPQGTFICEYVGE LISDAEADVREDDSYLFDLDNKDGEVYCIDARYYGNISRFINHLCDPNIIPVRVFMLHQDLRFP RIAFFSSRDIRTGEELGFDYGDRFWDIKSKYFTCQCGSEKCKHSAEAIALEQSRLARLDPH PELLPELGSLPPVN” or “RAPSRLQMFFANNHDQEFDPPKVYPPVPAEKRKPIRVLS
  MOD_RES (1457)..(2202): CONT. FROM ABOVE LFDGIATGLLVLKDLGIQVDRYIASEV CEDSITVGMVRHQGKIMYVGDVRSVTQKHIQEWGPFDLVIGGSPCNDLSIVNPARKGLYEGTGR LFFEFYRLLHDARPKEGDDRPFFWLFENVVAMGVSDKRDISRFLESNPVMIDAKEVSAAHR ARYFWGNLPGMNRPLASTVNDKLELQECLEHGRIAKFSKVRTITTRSNSIKQGKDQH
  MOD_RES (1457)..(2202): CONT. FROM ABOVE FPVFMNEKEDILWCTEMERVFGFPVHY TDVSNMSRLARQRLLGRSWSVPVIRHLFAPLKEYFACV” or “TLVTFKDVFVDFTREEWKLL DTAQQIVYRNVMLENYKNLVSLGYQLTKPDVILRLEKGEEPWLVEREIHQETHP”
  See specification as filed for detailed description of substitutions and preferred embodiments
MPKKKRKVGGSGGSDYKDHDGDYKDHDIDYKDDDDKGGGSGGGSGTGGSGGSGGSGGSGGSGRPMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQKKKRKVGLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGSGGSGGSGGSGGSASXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXTSGGGSGGGSKRPAATKKAGQAKKKKGGSGSGATNFSLLKQAGDVEENPGPAAA

SEQ ID NO: 11 - DNA, 60 bp, Artificial Sequence
  Description of Artificial Sequence Synthetic primer
ggtggcgggtccggcggtggatccggtaccggcagcgccgccatcgccgaagtccttctg

SEQ ID NO: 12 - DNA, 60 bp, Artificial Sequence
  Description of Artificial Sequence Synthetic primer
ggtggcgggtccggcggtggatccggtaccccacggcagaatctcaagtgtgtgcgtatc

SEQ ID NO: 13 - DNA, 54 bp, Artificial Sequence
  Description of Artificial Sequence Synthetic primer
ggtggcgggtccggcggtggatccggtaccatgactgaggatgtagacactcct

SEQ ID NO: 14 - DNA, 60 bp, Artificial Sequence
  Description of Artificial Sequence Synthetic primer
ggtggcgggtccggcggtggatccggtaccacactggtgaccttcaaggatgtatttgtg

SEQ ID NO: 15 - DNA, 54 bp, Artificial Sequence
  Description of Artificial Sequence Synthetic primer
ggtggcgggtccggcggtggatccggtacccgcgccccctcccggctccagatg

SEQ ID NO: 16 - DNA, 51 bp, Artificial Sequence
  Description of Artificial Sequence Synthetic primer
ggtggcgggtccggcggtggatccggtaccatgtccaggcggaaacagagc

SEQ ID NO: 17 - DNA, 60 bp, Artificial Sequence
  Description of Artificial Sequence Synthetic primer
accgccgcttccaccactccctccggtacttgtgttgacagggggcagggagccgagctc

SEQ ID NO: 18 - DNA, 59 bp, Artificial Sequence
  Description of Artificial Sequence Synthetic primer
accgccgcttccaccactccctccggtactgaagaggtatttgcggcaggactcagtcc

SEQ ID NO: 19 - DNA, 51 bp, Artificial Sequence
  Description of Artificial Sequence Synthetic primer
accgccgcttccaccactccctccggtactagggatttccatttctcgttc

SEQ ID NO: 20 - DNA, 60 bp, Artificial Sequence
  Description of Artificial Sequence Synthetic primer
accgccgcttccaccactccctccggtactaggatgggtctcttggtgaatttctctctc

SEQ ID NO: 21 - DNA, 54 bp, Artificial Sequence
  Description of Artificial Sequence Synthetic primer
accgccgcttccaccactccctccggtactcacacacgcaaaatactccttcag

SEQ ID NO: 22 - DNA, 50 bp, Artificial Sequence
  Description of Artificial Sequence Synthetic primer
accgccgcttccaccactccctccggtactagggctcggggcttcaggtg

SEQ ID NO: 23 - DNA, 39 bp, Artificial Sequence
  Description of Artificial Sequence Synthetic primer
gaattcatctcagaagcctgtggggagattatttctcag

SEQ ID NO: 24 - DNA, 44 bp, Artificial Sequence
  Description of Artificial Sequence Synthetic primer
ggtgaagagttgttttttgatttcagatacagccaggctgatgc

SEQ ID NO: 25 - DNA, 39 bp, Artificial Sequence
  Description of Artificial Sequence Synthetic primer
ctgagaaataatctccccacaggcttctgagatgaattc

SEQ ID NO: 26 - DNA, 44 bp, Artificial Sequence
  Description of Artificial Sequence Synthetic primer
gcatcagcctggctgtatctgaaatcaaaaaacaactcttcacc

SEQ ID NO: 27 - DNA, 60 bp, Artificial Sequence
  Description of Artificial Sequence Synthetic primer
ggatccggggggagcggagggagcgctagcggcagcgccgccatcgccgaagtccttctg

SEQ ID NO: 28 - DNA, 60 bp, Artificial Sequence
  Description of Artificial Sequence Synthetic primer
ggatccggggggagcggagggagcgctagcccacggcagaatctcaagtgtgtgcgtatc

SEQ ID NO: 29 - DNA, 51 bp, Artificial Sequence
  Description of Artificial Sequence Synthetic primer
ggatccggggggagcggagggagcgctagcatgtccaggcggaaacagagc

SEQ ID NO: 30 - DNA, 54 bp, Artificial Sequence
  Description of Artificial Sequence Synthetic primer
ggatccggggggagcggagggagcgctagccgcgccccctcccggctccagatg

SEQ ID NO: 31 - DNA, 59 bp, Artificial Sequence
  Description of Artificial Sequence Synthetic primer
ttagatccacctccggagcctccaccggatgtgttgacagggggcagggagccgagctc

SEQ ID NO: 32 - DNA, 59 bp, Artificial Sequence
  Description of Artificial Sequence Synthetic primer
ttagatccacctccggagcctccaccggagaagaggtatttgcggcaggactcagtccc

SEQ ID NO: 33 - DNA, 49 bp, Artificial Sequence
  Description of Artificial Sequence Synthetic primer
ttagatccacctccggagcctccaccggaagggctcggggcttcaggtg

SEQ ID NO: 34 - DNA, 53 bp, Artificial Sequence
  Description of Artificial Sequence Synthetic primer
ttagatccacctccggagcctccaccggacacacacgcaaaatactccttcag

SEQ ID NO: 35 - DNA, 42 bp, Artificial Sequence
  Description of Artificial Sequence Synthetic primer
gctaggtctctctatcggtaccatgtccaggcggaaacagag

SEQ ID NO: 36 - DNA, 35 bp, Artificial Sequence
  Description of Artificial Sequence Synthetic primer
gatggtctcgggtcgatgtccaggcggaaacagag

SEQ ID NO: 37 - DNA, 35 bp, Artificial Sequence
  Description of Artificial Sequence Synthetic primer
gatggtctcggctccatgtccaggcggaaacagag

SEQ ID NO: 38 - DNA, 35 bp, Artificial Sequence
  Description of Artificial Sequence Synthetic primer
gatggtctcggaagcatgtccaggcggaaacagag

SEQ ID NO: 39 - DNA, 35 bp, Artificial Sequence
  Description of Artificial Sequence Synthetic primer
gatggtctccgacccagggctcggggcttcaggtg

SEQ ID NO: 40 - DNA, 88 bp, Artificial Sequence
  Description of Artificial Sequence Synthetic primer
catggtctcacgccaggccggccgctgccgcctgagccaccagaaccgccgcttccaccactccctccagggctcggggcttcaggtg

SEQ ID NO: 41 - DNA, 35 bp, Artificial Sequence
  Description of Artificial Sequence Synthetic primer
gatggtctcggagccagggctcggggcttcaggtg

SEQ ID NO: 42 - DNA, 35 bp, Artificial Sequence
  Description of Artificial Sequence Synthetic primer
gatggtctcgcttccagggctcggggcttcaggtg

SEQ ID NO: 43 - DNA, 22 bp, Homo sapiens
gaatttatcccggactccgggg

SEQ ID NO: 44 - DNA, 22 bp, Homo sapiens
gttggaatgcagttggaggggg

SEQ ID NO: 45 - DNA, 22 bp, Homo sapiens
attccagaagatatgccccggg

SEQ ID NO: 46 - DNA, 27 bp, Homo sapiens
tttaagataaaacctgagacttaaaag

SEQ ID NO: 47 - DNA, 27 bp, Homo sapiens
tttctccctctcttcgcgcaggcctgg

SEQ ID NO: 48 - DNA, 27 bp, Homo sapiens
tttctccggtcccaatggaggggaatc

SEQ ID NO: 49 - DNA, 47 bp, Artificial Sequence
  Description of Artificial Sequence Synthetic primer
ggtggctcaggcggcagcggccggccaatgacacagttcgagggctt

SEQ ID NO: 50 - DNA, 30 bp, Artificial Sequence
  Description of Artificial Sequence Synthetic primer
tcggcatcgcccggggcgagagaaacctga

SEQ ID NO: 51 - DNA, 31 bp, Artificial Sequence
  Description of Artificial Sequence Synthetic primer
ctcgccccgggcgatgccgatgataggtgtc

SEQ ID NO: 52 - DNA, 89 bp, Artificial Sequence
  Description of Artificial Sequence Synthetic primer
cacctccggagcctccaccgctagcgctccctccgctccccccggatcctcctgaacctccactaccaccgttgcgcagctcctggatg

SEQ ID NO: 53 - DNA, 20 bp, Artificial Sequence
  Description of Artificial Sequence Synthetic primer
gggaaacctggaactcacct

SEQ ID NO: 54 - DNA, 20 bp, Artificial Sequence
  Description of Artificial Sequence Synthetic primer
gacctgcctcacttggttgt

SEQ ID NO: 55 - DNA, 20 bp, Artificial Sequence
  Description of Artificial Sequence Synthetic primer
ctggccgtaaactgctttgt

SEQ ID NO: 56 - DNA, 20 bp, Artificial Sequence
  Description of Artificial Sequence Synthetic primer
tcccaagttttgagccattc

SEQ ID NO: 57 - DNA, 22 bp, Artificial Sequence
  Description of Artificial Sequence Synthetic primer
aaacacaaacttgaacagctac

SEQ ID NO: 58 - DNA, 23 bp, Artificial Sequence
  Description of Artificial Sequence Synthetic primer
atttgaggcagtttacattatgg

SEQ ID NO: 59 - DNA, 20 bp, Artificial Sequence
  Description of Artificial Sequence Synthetic primer
aatcccatcaccatcttcca

SEQ ID NO: 60 - DNA, 19 bp, Artificial Sequence
  Description of Artificial Sequence Synthetic primer
ctccatggtggtgaagacg

SEQ ID NO: 61 - DNA, 20 bp, Artificial Sequence
  Description of Artificial Sequence Synthetic primer
ttggaatgcagttggagggg

SEQ ID NO: 62 - DNA, 20 bp, Artificial Sequence
  Description of Artificial Sequence Synthetic primer
ggtttctccggtcccaatgg

SEQ ID NO: 63 - DNA, 25 bp, Artificial Sequence
  Description of Artificial Sequence Synthetic primer
ggagggggtagagttattagttttt

SEQ ID NO: 64 - DNA, 25 bp, Artificial Sequence
  Description of Artificial Sequence Synthetic primer
aaataacaactcccaacttcacttt

SEQ ID NO: 65 - protein, 8 aa, Artificial Sequence
  Description of Artificial Sequence Synthetic peptide
DYKDDDDK

SEQ ID NO: 66 - protein, 22 aa, Artificial Sequence
  Description of Artificial Sequence Synthetic peptide
DYKDHDGDYKDHDIDYKDDDDK

SEQ ID NO: 67 - protein, 751 aa, Homo sapiens
MGQTGKKSEKGPVCWRKRVKSEYMRLRQLKRFRRADEVKSMFSSNRQKILERTEILNQEWKQRRIQPVHILTSVSSLRGTRECSVTSDLDFPTQVIPLKTLNAVASVPIMYSWSPLQQNFMVEDETVLHNIPYMGDEVLDQDGTFIEELIKNYDGKVHGDRECGFINDEIFVELVNALGQYNDDDDDDDGDDPEEREEKQKDLEDHRDDKESRPPRKFPSDKIFEAISSMFPDKGTAEELKEKYKELTEQQLPGALPPECTPNIDGPNAKSVQREQSLHSFHTLFCRRCFKYDCFLHRKCNYSFHATPNTYKRKNTETALDNKPCGPQCYQHLEGAKEFAAALTAERIKTPPKRPGGRRRGRLPNNSSRPSTPTINVLESKDTDSDREAGTETGGENNDKEEEEKKDETSSSSEANSRCQTPIKMKPNIEPPENVEWSGAEASMFRVLIGTYYDNFCAIARLIGTKTCRQVYEFRVKESSIIAPAPAEDVDTPPRKKKRKHRLWAAHCRKIQLKKDGSSNHVYNYQPCDHPRQPCDSSCPCVIAQNFCEKFCQCSSECQNRFPGCRCKAQCNTKQCPCYLAVRECDPDLCLTCGAADHWDSKNVSCKNCSIQRGSKKHLLLAPSDVAGWGIFIKDPVQKNEFISEYCGEIISQDEADRRGKVYDKYMCSFLFNLNNDFVVDATRKGNKIRFANHSVNPNCYAKVMMVNGDHRIGIFAKRAIQTGEELFFDYRYSQADALKYVGIEREMEIP

SEQ ID NO: 68 - protein, 7 aa, Simian virus 40
PKKKRKV

SEQ ID NO: 69 - protein, 8 aa, Simian virus 40
PKKKRKVG

SEQ ID NO: 70 - protein, 16 aa, Unknown
  Description of Unknown Bipartite NLS sequence
KRPAATKKAGQAKKKK

SEQ ID NO: 71 - protein, 3 aa, Artificial Sequence
  Description of Artificial Sequence Synthetic peptide
GGS

SEQ ID NO: 72 - protein, 6 aa, Artificial Sequence
  Description of Artificial Sequence Synthetic peptide
GGSGGS

SEQ ID NO: 73 - protein, 9 aa, Artificial Sequence
  Description of Artificial Sequence Synthetic peptide
GGSGGSGGS

SEQ ID NO: 74 - protein, 12 aa, Artificial Sequence
  Description of Artificial Sequence Synthetic peptide
GGSGGSGGSGGS

SEQ ID NO: 75 - protein, 15 aa, Artificial Sequence
  Description of Artificial Sequence Synthetic peptide
GGSGGSGGSGGSGGS

SEQ ID NO: 76 - protein, 18 aa, Artificial Sequence
  Description of Artificial Sequence Synthetic peptide
GGSGGSGGSGGSGGSGGS

SEQ ID NO: 77 - protein, 21 aa, Artificial Sequence
  Description of Artificial Sequence Synthetic peptide
GGSGGSGGSGGSGGSGGSGGS

SEQ ID NO: 78 - protein, 24 aa, Artificial Sequence
  Description of Artificial Sequence Synthetic peptide
GGSGGSGGSGGSGGSGGSGGSGGS

SEQ ID NO: 79 - protein, 27 aa, Artificial Sequence
  Description of Artificial Sequence Synthetic peptide
GGSGGSGGSGGSGGSGGSGGSGGSGGS

SEQ ID NO: 80 - protein, 30 aa, Artificial Sequence
  Description of Artificial Sequence Synthetic polypeptide
GGSGGSGGSGGSGGSGGSGGSGGSGGSGGS

SEQ ID NO: 81 - protein, 2256 aa, Artificial Sequence
  Description of Artificial Sequence Synthetic polypeptide
MPKKKRKVGGSGGSDYKDHDGDYKDHDIDYKDDDDKGGGSGGGSGTMGQTGKKSEKGPVCWRKRVKSEYMRLRQLKRFRRADEVKTMFSSNRQKILERTETLNQEWKQRRIQPVHIMTSVSSLRGTRECSVTSDLDFPAQVIPLKTLNAVASVPIMYSWSPLQQNFMVEDETVLHNIPYMGDEVLDQDGTFIEELIKNYDGKVHGDRECGFINDEIFVELVNALGQYNDDDDDDDGDDPDEREEKQKDLEDNRDDKETCPPRKFPADKIFEAISSMFPDKGTAEELKEKYKELTEQQLPGALPPECTPNIDGPNAKSVQREQSLHSFHTLFCRRCFKYDCFLHPFHATPNTYKRKNTETALDNKPCGPQCYQHLEGAKEFAAALTAERIKTPPKRPGGRRRGRLPNNSSRPSTPTISVLESKDTDSDREAGTETGGENNDKEEEEKKDETSSSSEANSRCQTPIKMKPNIEPPENVEWSGAEASMFRVLIGTYYDNFCAIARLIGTKTCRQVYEFRVKESSIIAPVPTEDVDTPPRKKKRKHRLWAAHCRKIQLKKDGSSNHVYNYQPCDHPRQPCDSSCPCVIAQNFCEKFCQCSSECQNRFPGCRCKAQCNTKQCPCYLAVRECDPDLCLTCGAADHWDSKNVSCKNCSIQRGSKKHLLLAPSDVAGWGIFIKDPVQKNEFISEYCGEIISQDEADRRGKVYDKYMCSFLFNLNNDFVVDATRKGNKIRFANHSVNPNCYAKVMMVNGDHRIGIFAKRAIQTGEELFFDYRYSQADALKYVGIEREMEIPSTGGSGGSGGSGGSGGSGRPMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQKKKRKVGLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGSGGSGGSGGSGGSASGGGSGGGSKRPAATKKAGQAKKKKGGSGSGATNFSLLKQAGDVEENPGPAAA

SEQ ID NO: 82 - protein, 1775 aa, Artificial Sequence
  Description of Artificial Sequence Synthetic polypeptide
MPKKKRKVGGSGGSDYKDHDGDYKDHDIDYKDDDDKGGGSGGGSGTTEDVDTPPRKKKRKHRLWAAHCRKIQLKKDGSSNHVYNYQPCDHPRQPCDSSCPCVIAQNFCEKFCQCSSECQNRFPGCRCKAQCNTKQCPCYLAVRECDPDLCLTCGAADHWDSKNVSCKNCSIQRGSKKHLLLAPSDVAGWGIFIKDPVQKNEFISEYCGEIISQDEADRRGKVYDKYMCSFLFNLNNDFVVDATRKGNKIRFANHSVNPNCYAKVMMVNGDHRIGIFAKRAIQTGEELFFDYRYSQADALKYVGIEREMEIPSTGGSGGSGGSGGSGGSGRPMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQKKKRKVGLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGSGGSGGSGGSGGSASGGGSGGGSKRPAATKKAGQAKKKKGGSGSGATNFSLLKQAGDVEENPGPAAA

SEQ ID NO: 83 - protein, 1555 aa, Artificial Sequence
  Description of Artificial Sequence Synthetic polypeptide
MPKKKRKVGGSGGSDYKDHDGDYKDHDIDYKDDDDKGGGSGGGSGTMSRRKQSNPRQIKRSLGDMEAREEVQLVGASHMEQKATAPEAPSPSTGGSGGSGGSGGSGGSGRPMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQKKKRKVGLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGSGGSGGSGGSGGSASGGGSGGGSKRPAATKKAGQAKKKKGGSGSGATNFSLLKQAGDVEENPGPAAA

SEQ ID NO: 84 - protein, 1847 aa, Artificial Sequence
  Description of Artificial Sequence Synthetic polypeptide
MPKKKRKVGGSGGSDYKDHDGDYKDHDIDYKDDDDKGGGSGGGSGTPRQNLKCVRILKQFHKDLERELLRRHHRSKTPRHLDPSLANYLVQKAKQRRALRRWEQELNAKRSHLGRITVENEVDLDGPPRAFVYINEYRVGEGITLNQVAVGCECQDCLWAPTGGCCPGASLHKFAYNDQGQVRLRAGLPIYECNSRCRCGYDCPNRVVQKGIRYDLCIFRTDDGRGWGVRTLEKIRKNSFVMEYVGEIITSEEAERRGQIYDRQGATYLFDLDYVEDVYTVDAAYYGNISHFVNHSCDPNLQVYNVFIDNLDERLPRIAFFATRTIRAGEELTFDYNMQVDPVDMESTRMDSNFGLAGLPGSPKKRVRIECKCGTESCRKYLFSTGGSGGSGGSGGSGGSGRPMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQKKKRKVGLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGSGGSGGSGGSGGSASGGGSGGGSKRPAATKKAGQAKKKKGGSGSGATNFSLLKQAGDVEENPGPAAA

SEQ ID NO: 85 - protein, 1891 aa, Artificial Sequence
  Description of Artificial Sequence Synthetic polypeptide
MPKKKRKVGGSGGSDYKDHDGDYKDHDIDYKDDDDKGGGSGGGSGTGSAAIAEVLLNARCDLHAVNYHGDTPLHIAARESYHDCVLLFLSRGANPELRNKEGDTAWDLTPERSDVWFALQLNRKLRLGVGNRAIRTEKIICRDVARGYENVPIPCVNGVDGEPCPEDYKYISENCETSTMNIDRNITHLQHCTCVDDCSSSNCLCGQLSIRCWYDKDGRLLQEFNKIEPPLIFECNQACSCWRNCKNRVVQSGIKVRLQLYRTAKMGWGVRALQTIPQGTFICEYVGELISDAEADVREDDSYLFDLDNKDGEVYCIDARYYGNISRFINHLCDPNIIPVRVFMLHQDLRFPRIAFFSSRDIRTGEELGFDYGDRFWDIKSKYFTCQCGSEKCKHSAEAIALEQSRLARLDPHPELLPELGSLPPVNSTGGSGGSGGSGGSGGSGRPMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQKKKRKVGLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGSGGSGGSGGSGGSASGGGSGGGSKRPAATKKAGQAKKKKGGSGSGATNFSLLKQAGDVEENPGPAAA

SEQ ID NO: 86 - protein, 1823 aa, Artificial Sequence
  Description of Artificial Sequence Synthetic polypeptide
MPKKKRKVGGSGGSDYKDHDGDYKDHDIDYKDDDDKGGGSGGGSGTRAPSRLQMFFANNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSTGGSGGSGGSGGSGGSGRPMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQKKKRKVGLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGSGGSGGSGGSGGSASGGGSGGGSKRPAATKKAGQAKKKKGGSGSGATNFSLLKQAGDVEENPGPAAA

SEQ ID NO: 87 - protein, 1584 aa, Artificial Sequence
  Description of Artificial Sequence Synthetic polypeptide
MPKKKRKVGGSGGSDYKDHDGDYKDHDIDYKDDDDKGGGSGGGSGTTLVTFKDVFVDFTREEWKLLDTAQQIVYRNVMLENYKNLVSLGYQLTKPDVILRLEKGEEPWLVEREIHQETHPSTGGSGGSGGSGGSGGSGRPMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQKKKRKVGLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGSGGSGGSGGSGGSASGGGSGGGSKRPAATKKAGQAKKKKGGSGSGATNFSLLKQAGDVEENPGPAAA

SEQ ID NO: 88 - protein, 2256 aa, Artificial Sequence
  Description of Artificial Sequence Synthetic polypeptide
MPKKKRKVGGSGGSDYKDHDGDYKDHDIDYKDDDDKGGGSGGGSGTGGSGGSGGSGGSGGSGRPMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQKKKRKVGLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGSGGSGGSGGSGGSASMGQTGKKSEKGPVCWRKRVKSEYMRLRQLKRFRRADEVKTMFSSNRQKILERTETLNQEWKQRRIQPVHIMTSVSSLRGTRECSVTSDLDFPAQVIPLKTLNAVASVPIMYSWSPLQQNFMVEDETVLHNIPYMGDEVLDQDGTFIEELIKNYDGKVHGDRECGFINDEIFVELVNALGQYNDDDDDDDGDDPDEREEKQKDLEDNRDDKETCPPRKFPADKIFEAISSMFPDKGTAEELKEKYKELTEQQLPGALPPECTPNIDGPNAKSVQREQSLHSFHTLFCRRCFKYDCFLHPFHATPNTYKRKNTETALDNKPCGPQCYQHLEGAKEFAAALTAERIKTPPKRPGGRRRGRLPNNSSRPSTPTISVLESKDTDSDREAGTETGGENNDKEEEEKKDETSSSSEANSRCQTPIKMKPNIEPPENVEWSGAEASMFRVLIGTYYDNFCAIARLIGTKTCRQVYEFRVKESSIIAPVPTEDVDTPPRKKKRKHRLWAAHCRKIQLKKDGSSNHVYNYQPCDHPRQPCDSSCPCVIAQNFCEKFCQCSSECQNRFPGCRCKAQCNTKQCPCYLAVRECDPDLCLTCGAADHWDSKNVSCKNCSIQRGSKKHLLLAPSDVAGWGIFIKDPVQKNEFISEYCGEIISQDEADRRGKVYDKYMCSFLFNLNNDFVVDATRKGNKIRFANHSVNPNCYAKVMMVNGDHRIGIFAKRAIQTGEELFFDYRYSQADALKYVGIEREMEIPTSGGGSGGGSKRPAATKKAGQAKKKKGGSGSGATNFSLLKQAGDVEENPGPAAA

SEQ ID NO: 89 - protein, 1775 aa, Artificial Sequence
  Description of Artificial Sequence Synthetic polypeptide
MPKKKRKVGGSGGSDYKDHDGDYKDHDIDYKDDDDKGGGSGGGSGTGGSGGSGGSGGSGGSGRPMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQKKKRKVGLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGSGGSGGSGGSGGSASTEDVDTPPRKKKRKHRLWAAHCRKIQLKKDGSSNHVYNYQPCDHPRQPCDSSCPCVIAQNFCEKFCQCSSECQNRFPGCRCKAQCNTKQCPCYLAVRECDPDLCLTCGAADHWDSKNVSCKNCSIQRGSKKHLLLAPSDVAGWGIFIKDPVQKNEFISEYCGEIISQDEADRRGKVYDKYMCSFLFNLNNDFVVDATRKGNKIRFANHSVNPNCYAKVMMVNGDHRIGIFAKRAIQTGEELFFDYRYSQADALKYVGIEREMEIPTSGGGSGGGSKRPAATKKAGQAKKKKGGSGSGATNFSLLKQAGDVEENPGPAAA

SEQ ID NO: 90 - protein, 1555 aa, Artificial Sequence
  Description of Artificial Sequence Synthetic polypeptide
MPKKKRKVGGSGGSDYKDHDGDYKDHDIDYKDDDDKGGGSGGGSGTGGSGGSGGSGGSGGSGRPMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQKKKRKVGLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGSGGSGGSGGSGGSASMSRRKQSNPRQIKRSLGDMEAREEVQLVGASHMEQKATAPEAPSPTSGGGSGGGSKRPAATKKAGQAKKKKGGSGSGATNFSLLKQAGDVEENPGPAAA

SEQ ID NO: 91 - protein, 1847 aa, Artificial Sequence
  Description of Artificial Sequence Synthetic polypeptide
MPKKKRKVGGSGGSDYKDHDGDYKDHDIDYKDDDDKGGGSGGGSGTGGSGGSGGSGGSGGSGRPMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQKKKRKVGLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGSGGSGGSGGSGGSASPRQNLKCVRILKQFHKDLERELLRRHHRSKTPRHLDPSLANYLVQKAKQRRALRRWEQELNAKRSHLGRITVENEVDLDGPPRAFVYINEYRVGEGITLNQVAVGCECQDCLWAPTGGCCPGASLHKFAYNDQGQVRLRAGLPIYECNSRCRCGYDCPNRVVQKGIRYDLCIFRTDDGRGWGVRTLEKIRKNSFVMEYVGEIITSEEAERRGQIYDRQGATYLFDLDYVEDVYTVDAAYYGNISHFVNHSCDPNLQVYNVFIDNLDERLPRIAFFATRTIRAGEELTFDYNMQVDPVDMESTRMDSNFGLAGLPGSPKKRVRIECKCGTESCRKYLFTSGGGSGGGSKRPAATKKAGQAKKKKGGSGSGATNFSLLKQAGDVEENPGPAAA

SEQ ID NO: 92 - protein, 1891 aa, Artificial Sequence
  Description of Artificial Sequence Synthetic polypeptide
MPKKKRKVGGSGGSDYKDHDGDYKDHDIDYKDDDDKGGGSGGGSGTGGSGGSGGSGGSGGSGRPMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQKKKRKVGLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGSGGSGGSGGSGGSASGSAAIAEVLLNARCDLHAVNYHGDTPLHIAARESYHDCVLLFLSRGANPELRNKEGDTAWDLTPERSDVWFALQLNRKLRLGVGNRAIRTEKIICRDVARGYENVPIPCVNGVDGEPCPEDYKYISENCETSTMNIDRNITHLQHCTCVDDCSSSNCLCGQLSIRCWYDKDGRLLQEFNKIEPPLIFECNQACSCWRNCKNRVVQSGIKVRLQLYRTAKMGWGVRALQTIPQGTFICEYVGELISDAEADVREDDSYLFDLDNKDGEVYCIDARYYGNISRFINHLCDPNIIPVRVFMLHQDLRFPRIAFFSSRDIRTGEELGFDYGDRFWDIKSKYFTCQCGSEKCKHSAEAIALEQSRLARLDPHPELLPELGSLPPVNTSGGGSGGGSKRPAATKKAGQAKKKKGGSGSGATNFSLLKQAGDVEENPGPAAA

SEQ ID NO: 93 - protein, 1823 aa, Artificial Sequence
  Description of Artificial Sequence Synthetic polypeptide
MPKKKRKVGGSGGSDYKDHDGDYKDHDIDYKDDDDKGGGSGGGSGTGGSGGSGGSGGSGGSGRPMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQKKKRKVGLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGSGGSGGSGGSGGSASRAPSRLQMFFANNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVTSGGGSGGGSKRPAATKKAGQAKKKKGGSGSGATNFSLLKQAGDVEENPGPAAA

SEQ ID NO: 94 - protein, 1584 aa, Artificial Sequence
  Description of Artificial Sequence Synthetic polypeptide
MPKKKRKVGGSGGSDYKDHDGDYKDHDIDYKDDDDKGGGSGGGSGTGGSGGSGGSGGSGGSGRPMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQKKKRKVGLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGSGGSGGSGGSGGSASTLVTFKDVFVDFTREEWKLLDTAQQIVYRNVMLENYKNLVSLGYQLTKPDVILRLEKGEEPWLVEREIHQETHPTSGGGSGGGSKRPAATKKAGQAKKKKGGSGSGATNFSLLKQAGDVEENPGPAAA

SEQ ID NO: 95 - protein, 30 aa, Artificial Sequence; identical to SEQ ID NO: 80
  Description of Artificial Sequence Synthetic polypeptide
  MISC_FEATURE (1)..(30): This sequence may encompass 1-10 “Gly Gly Ser” repeating units

SEQ ID NO: 96 - protein, 10 aa, Artificial Sequence
  Description of Artificial Sequence Synthetic peptide
  MISC_FEATURE (1)..(10): This sequence may encompass 6-10 residues
HHHHHHHHHH

(19) United States
(12) Patent Application Publication
(10) Pub. No.: US 2019/0233805 A1
(43) Pub. Date: Aug. 1, 2019

(54) TARGETABLE PROTEINS FOR EPIGENETIC MODIFICATION AND METHODS FOR USE THEREOF

(71) Applicant: The Regents of the University of California, Oakland, CA (US)

(72) Inventors: David J Segal, Davis, CA (US); Henriette O'Geen, Davis, CA (US); Joel P. Mackay, Sydney (AU); Peggy J. Farnham, Pasadena, CA (US)

(73) Assignee: The Regents of the University of California, Oakland, CA (US)

(21) Appl. No.: 16/151,895

(22) Filed: Oct. 4, 2018

Related U.S. Application Data
(60) Provisional application No. 62/568,156, filed on Oct. 4, 2017.

(51) Int. Cl. C12N 9/22; C12N 9/10; C07K 14/47

(52) U.S. Cl. CPC C12N 9/22; C12N 9/1007; C12Y 201/01043; C07K 2319/43; C07K 14/4702; C07K 2319/81; C07K 2319/09; C12Y 201/01037

(57) ABSTRACT

Provided herein are fusion proteins comprising a catalytically inactive Cas9 domain and an effector domain. The fusion proteins of the present invention can be used to, for example, produce epigenetic modifications at target chromatin sites. Nucleic acids and expression vectors encoding the fusion proteins, as well as cells comprising the fusion proteins, are also provided herein.

### CROSS-REFERENCE TO RELATED APPLICATIONS

The present application claims priority to U.S. Provisional Application No. 62/568,156, filed Oct. 4, 2017, the disclosure of which is herein incorporated by reference in its entirety for all purposes.

### STATEMENT AS TO RIGHTS TO INVENTIONS MADE UNDER FEDERALLY SPONSORED RESEARCH AND DEVELOPMENT

This invention was made with Government support under Grant No. CA204563, awarded by the National Institutes of Health. The Government has certain rights in this invention.

### REFERENCE TO SUBMISSION OF A SEQUENCE LISTING AS A TEXT FILE

The Sequence Listing written in file 081906-226310US-1106551_SL.txt created on Dec. 6, 2018, 325,708 bytes, machine format IBM-PC, MS-Windows operating system, is hereby incorporated by reference in its entirety for all purposes.

### BACKGROUND OF THE INVENTION

While genomic DNA holds the key to the genetic code, epigenetics offers another layer of information that establishes cell fate during development, aging and disease, as well as in response to the environment. Epigenetics is a means by which the transcriptome (and thus the proteome) of a cell can be changed without alteration of the genetic content. Epigenetic regulation is thought to be accomplished through epigenetic marks such as posttranslational modifications of histones and DNA methylation, and also via other mechanisms involving noncoding RNAs (1,2). Regions of active gene expression and open chromatin carry a signature of epigenetic marks that is distinct from repressed and heterochromatic regions (2). For example, histone acetylation is always associated with active transcription, while different histone methylation marks are associated with active versus repressed chromatin. Specifically, trimethylation of lysine 4 on histone H3 (H3K4me3) is associated with active transcription, while trimethylation of H3K9 (H3K9me3) and H3K27 (H3K27me3) are associated with repressed chromatin regions. There has been a significant effort to decipher the relationship between epigenetic marks, regulatory element activity and gene regulation. Large consortia projects such as ENCODE and the Roadmap Epigenomics Project have mapped epigenetic signatures across the human genome in many different human cell types and tissues, which have then been correlated with gene expression (3,4). These association-based studies have provided epigenomic landscapes of epigenetic marks present at promoters and other regulatory elements, but cannot dissect the dynamic relationships between the epigenome and transcriptional control. While some evidence suggests that silencing of gene expression precedes de novo DNA methylation (5), the causal relationship between the presence of a histone mark and gene expression is still unclear. Accordingly, there is a need in the art for new tools that can be used to further explore the relationships between epigenetic modifications, transcriptional control, organismal development, and disease states. The present invention satisfies this need, and provides related advantages as well.

### BRIEF SUMMARY OF THE INVENTION

In one aspect, the present invention provides a fusion protein comprising (1) a catalytically inactive Cas9 (dCas9) domain and (2) an effector domain, wherein the effector domain is enhancer of zeste homolog 2 (Ezh2), Friend of GATA1 (FOG1), histone H3 lysine 9 methyltransferase G9A (G9A), histone-lysine N-methyltransferase SUV39H1 (SUV39H1), Krüppel-associated box (KRAB), DNA (cytosine-5)-methyltransferase 3A (DNMT3A), or a combination thereof. In some embodiments, the effector domain is located N-terminal and/or C-terminal to the dCas9 domain. In some embodiments, the fusion protein further comprises a nuclear localization signal (NLS) domain, a FLAG epitope tag, an amino acid linker, or a combination thereof. In some embodiments, the NLS domain, the FLAG epitope tag, and/or the amino acid linker are located N-terminal and/or C-terminal to the dCas9 domain. In some instances, the amino acid linker comprises the amino acid sequence (GGS)ₙ, wherein the subscript n is the number of repeat units and is between 1 and 10 (e.g., n is equal to 1, 2, 3, 4, 5, 6, 7, 8, 9, or 10) (SEQ ID NO: 95). In some embodiments, the amino acid linker sequence comprises the amino acid sequence of any one of SEQ ID NOS:71-80. In particular embodiments, the effector domain is KRAB or DNMT3A and the effector domain is located N-terminal to the dCas9 domain.

In some embodiments, the effector domain is Ezh2 and the Ezh2 effector domain comprises the conserved cysteine-rich (CXC) and Su(var)3-9, Enhancer-of-zeste and Trithorax (SET) domains. In some embodiments, the Ezh2 effector domain further comprises the embryonic ectoderm development (EED) binding domain. In some embodiments, the Ezh2 effector domain comprises amino acids 1-746 of Ezh2 (SEQ ID NO:1). In some instances, the Ezh2 effector domain is located N-terminal to the dCas9 domain.

In some embodiments, the effector domain comprises amino acids 1-45 of FOG1 (SEQ ID NO:3), a first NLS domain is located at the N-terminal end of the protein, and a second NLS domain is located at the C-terminal end of the protein. In particular embodiments, the fusion protein further comprises a FLAG epitope tag that is located between the first NLS domain and the N-terminal end of the dCas9 domain.

In some embodiments, the FOG1 effector domain comprises 1, 2, 3, or 4 FOG1 effector domains that are located between the FLAG epitope tag and the N-terminal end of the dCas9 domain. In particular embodiments, the fusion protein further comprises an amino acid linker comprising the amino acid sequence (GGS)ₙ, wherein n is 5 (SEQ ID NO:75), and the amino acid linker is located between the FOG1 effector domain and the N-terminal end of the dCas9 domain. In some embodiments, the fusion protein further comprises an amino acid linker comprising the amino acid sequence (GGS)ₙ, wherein n is 5 (SEQ ID NO:75), and the amino acid linker is located between the C-terminal end of the dCas9 domain and the second NLS domain.

In some embodiments, the FOG1 effector domain is located between the second NLS domain and the C-terminal end of the dCas9 domain. In particular embodiments, the fusion protein further comprises an amino acid linker comprising the amino acid sequence (GGS)ₙ, wherein n is 5 (SEQ ID NO:75), and the amino acid linker is located between the FLAG epitope tag and the N-terminal end of the dCas9 domain. In some embodiments, the fusion protein further comprises an amino acid linker comprising the amino acid sequence (GGS)ₙ, wherein n is 5 (SEQ ID NO:75), and the amino acid linker is located between the C-terminal end of the dCas9 domain and the FOG1 effector domain.

In some embodiments, a first FOG1 effector domain is located between the FLAG epitope tag and the N-terminal end of the dCas9 domain, and a second FOG1 effector domain is located between the C-terminal end of the dCas9 domain and the second NLS domain. In particular embodiments, the fusion protein further comprises an amino acid linker comprising the amino acid sequence (GGS)ₙ, wherein n is 5 (SEQ ID NO:75), and the amino acid linker is located between the first FOG1 effector domain and the N-terminal end of the dCas9 domain. In some embodiments, the fusion protein further comprises an amino acid linker comprising the amino acid sequence (GGS)ₙ, wherein n is 5 (SEQ ID NO:75), and the amino acid linker is located between the C-terminal end of the dCas9 domain and the second FOG1 effector domain.

In another aspect, the present invention provides a nucleic acid comprising a polynucleotide sequence encoding a fusion protein provided herein. In yet another aspect, the present invention provides an expression vector comprising a nucleic acid provided herein. In still another aspect, the present invention provides a cell comprising a fusion protein provided herein or an expression vector provided herein.

In another aspect, the present invention provides a method for producing an epigenetic modification of a target chromatin site comprising a Cas9 recognition site. In some embodiments, the method comprises contacting the target chromatin site with a fusion protein provided herein. In particular embodiments, the epigenetic modification comprises acetylation, deacetylation, methylation, or a combination thereof. In some instances, methylation comprises the addition of one, two, or three methyl groups.

In some embodiments, an epigenetic modification of a nucleic acid and/or a histone protein is produced. In particular embodiments, an epigenetic modification of histone H3 is produced. In some instances, lysine 9 on histone H3 is trimethylated (H3K9me3) and/or lysine 27 on histone H3 is trimethylated (H3K27me3).

In some embodiments, an epigenetic modification is produced in vitro. In other embodiments, the fusion protein and the target chromatin site are in a cell. In some embodiments, the method further comprises contacting the target chromatin site with a single guide RNA (sgRNA). In particular embodiments, expression of the target chromatin site is suppressed.

Other objects, features, and advantages of the present invention will be apparent to one of skill in the art from the following detailed description and figures.

### BRIEF DESCRIPTION OF THE DRAWINGS

FIGS. 1A and 1B show a single-strand annealing (SSA) reporter assay for evaluation of Cpf1 and dCpf1 cleavage ability. FIG. 1A shows a schematic of the SSA assay. The crRNA binding region of HER2 promoter was inserted between direct repeats to interrupt the mCherry gene. Double strand cleavage by an active nuclease induced the SSA pathway, leading to functional mCherry expression. FIG. 1B shows fluorescent detection of nuclease activity. HEK293T cells were co-transfected with mCherry reporter plasmid, PCR amplified U6-crRNA cassette, and either Cpf1- or dCpf1-expressing plasmid. Targeted Cpf1 cleavage activity resulted in joining of the split mCherry gene and red fluorescence. Red fluorescence was evaluated 48 hours post transfection. Transfection with reporter plasmid and Cpf1-expressing plasmid (but without crRNA) did not result in red fluorescence (left panel), but Cpf1 together with HER2 crRNA1 specifically cleaved the reporter plasmid leading to mCherry expression (middle panel). No fluorescence was observed when using the catalytically inactive dCpf1 (right panel).

FIGS. 2A-2F show that N-terminal fusions of H3K9 methyltransferases to dCas9 repressed HER2 gene expression independent of histone methylation. FIG. 2A shows a schematic representation of the H3K9 histone methyltransferases G9A and SUV39H1, with protein domains indicated. Regions that were fused to dCas9 are labeled as G9A[SET] and SUV[SET]. FIG. 2B shows the genomic target sites of the three sgRNAs that targeted dCas9 to a 500-bp region of the HER2 promoter (vertical bars). ENCODE tracks of DNaseHS, H3K4me3, and H3K27A in HCT116 are shown. FIG. 2C shows the design of dCas9 fusion proteins. dCas9 fusions contained N-terminal and C-terminal nuclear localization domains (NLSs), as well as an N-terminal 3xFLAG epitope tag. dCas9 fusion proteins contained the histone methyltransferase effector domain (ED) at the N-terminus, C-terminus, or both the N- and C-termini (labeled [N], [C], and [N+C], respectively). A 15-amino acid linker ((GGS)₅) (SEQ ID NO:75) separated the dCas9 and the EDs. FIG. 2D shows the relative HER2 mRNA levels resulting from dCas9-ED fusions compared to dCas9 with no ED, as determined by RT-qPCR in HCT116 cells after co-transfection of plasmids expressing the indicated dCas9 fusions with the three sgRNAs targeted to the HER2 promoter (Tukey test, P<0.01, n=2 independent experiments each; mean±SEM). FIG. 2E shows H3K9me3 ChIP-qPCR enrichment at the HER2 promoter in HCT116 cells co-transfected with three sgRNAs targeted to the HER2 promoter and the indicated N-terminal dCas9 fusions (Tukey test, *P<0.05, n=2 independent experiments; mean±SD). The number above the bar indicates the fold-increase in H3K9me3 enrichment relative to dCas9 with no ED. ChIP assays using normal rabbit IgG were used as negative controls. FIG. 2F shows ChIP-qPCR enrichment of H3K27me3 and H3K9me2 at the HER2 promoter in HCT116 cells co-transfected with three sgRNAs and the indicated dCas9 fusions (No comparisons were significant, n=2 independent experiments; mean±SD).

FIGS. 3A-3C show Western blot analysis of protein levels of various dCas9 fusions expressed in HCT116 cells. FIG. 3A shows expression of dCas9 fusions with indicated G9A or SUV SET domains at the N-terminus [N], C-terminus [C], or both [N+C]. Ponceau S staining was provided as a loading control. Corresponding activity assays are shown in FIG. 2D. FIG. 3B shows expression of dCas9 fusions with indicated FOG1[1-45] domains. Corresponding activity assays are shown in FIG. 5C. Ponceau S staining was provided as a loading control. FIG. 3C shows expression of dCas9 fusions with indicated effector domains. Corresponding activity assays are shown in FIG. 7A. Beta-actin staining was provided as a loading control. dCas9-fusion proteins were detected using an anti-FLAG antibody (1:1000; SIGMA M2 F1804), and beta-actin by an anti-beta-actin antibody (1:2500; SIGMA A5441), as described in the Methods and Materials section of Example 1.

FIGS. 4A-4F show that N-terminal fusions of Ezh2 H3K27 methyltransferases to dCas9 repressed HER2 gene expression independent of histone methylation. FIG. 4A shows a schematic representation of the H3K27 methyltransferase Ezh2. Regions of each protein fused to dCas9 are labeled Ezh2[SET] and Ezh2[FL], and protein domains are indicated. FIG. 4B shows relative HER2 mRNA production in cells co-transfected with a pool of three sgRNAs targeted to the HER2 gene promoter and the indicated dCas9 fusions. Expression data are shown in comparison to cells transfected by dCas9 with no ED (Tukey test, *P<0.05, **P<0.01, n=2 independent experiments; mean±SEM). FIG. 4C shows H3K27me3 enrichment, assessed for the indicated dCas9 fusion proteins as in FIG. 2 (Tukey test, P<0.01, n=2 independent experiments; mean±SD). FIG. 4D shows ChIP-qPCR enrichment of H3K9me2 and H3K9me3 at the HER2 promoter in HCT116 cells, as in FIG. 2F. FIG. 4E shows a schematic representation of Ezh2[SET] catalytic mutants. FIG. 4F shows relative HER2 mRNA production using the indicated Ezh2[SET]-dCas9 fusions. Expression data are shown in comparison to cells transfected by dCas9 with no ED (ns, not significant; n=2 independent experiments; mean±SEM).

FIGS. 5A-5D show that the novel transcriptional repressor FOG1[1-45]-dCas9-FOG1[1-45] trimethylated H3K27 at the target promoter. FIG. 5A shows models for two approaches of targeted H3K27 methylation mediated by dCas9-fusion proteins. Top: Fusion of dCas9 to the enzyme Ezh2 directly trimethylates H3K27 at the genomic target region. Bottom: Fusion of dCas9 to subunits or interaction domains of endogenous co-repressor complexes, such as FOG1[1-45]-dCas9 that interacts with the NuRD complex, recruits the NuRD complex to the target sites causing HDAC1/2-mediated H3K27 deacetylation, as well as facilitation of H3K27 trimethylation through recruitment of the PRC2 complex. FIG. 5B shows a schematic of dCas9-FOG1[1-45] fusion proteins. Fusions to the N- and/or C-termini of dCas9 are labeled with [N] and/or [C], respectively. Arrays of two, three, and four FOG1[1-45] repeats were fused to dCas9. Nuclear localization signals (NLSs), 3× FLAG epitope tag, and the 15-amino acid linkers ((GGS)₅) (SEQ ID NO:75) are indicated. FIG. 5C shows relative HER2 mRNA levels, as assessed in HCT116 cells co-transfected with a pool of three sgRNAs targeted to the HER2 promoter and the indicated dCas9-FOG1[1-45] fusions. Repressive activity was measured relative to Cas9 with no ED (Tukey test, *P<0.05, **P<0.01, n=2 independent experiments; mean±SEM). Negative control cells (“−”) were transfected with mCherry reporter plasmid instead of dCas9. FIG. 5D shows H3K27ac and H3K27me3 enrichments that were assessed by ChIP-qPCR at the HER2 promoter after transfection with a dCas9 with no ED or FOG1[1-45]-dCas9-FOG1[1-45] (Tukey test, ns, not significant; **P<0.01; n=2 independent experiments; mean±SD). ChIP assays using normal rabbit IgG were used as negative controls.

FIGS. 6A and 6B show that KRAB-dCas9 and DNMT3A-dCas9 deposited their expected epigenetic marks. FIG. 6A shows H3K9me3 and H3K27me3 ChIP-qPCR enrichment at the HER2 promoter in HCT116 cells co-transfected with three sgRNAs targeted to the HER2 promoter and dCas9 with no ED or KRAB-dCas9 (Tukey test, *P<0.05, n=2 independent experiments; mean±SEM). ChIP assays using normal rabbit IgG were used as negative controls. FIG. 6B shows targeted DNA methylation analysis. HCT116 cells were co-transfected with plasmids expressing DNMT3A-dCas9 and three sgRNAs targeting the HER2 promoter. After bisulfite conversion, a 150 bp region of the HER2 promoter was amplified, cloned, and sequenced. Each line represents a single clone with circles indicating CpG nucleotides (empty circles denote unmethylated, filled circles denote methylated). Untreated cells were used as a negative control.

FIGS. 7A-7F show variable repression mediated by ED-dCas9 epigenetic modifiers at three loci in two cell types. FIG. 7A shows relative mRNA production in HCT116 cells co-transfected with a pool of three sgRNAs targeted to the HER2 promoter with the indicated dCas9 fusions. FIG. 7B shows relative mRNA production in HEK293T cells co-transfected with a pool of three sgRNAs targeted to the HER2 promoter with the indicated dCas9 fusions. FIG. 7C shows relative mRNA production in HCT116 cells co-transfected with a pool of three sgRNAs targeted to the MYC promoter with the indicated dCas9 fusions. FIG. 7D shows relative mRNA production in HEK293T cells co-transfected with a pool of three sgRNAs targeted to the MYC promoter with the indicated dCas9 fusions. FIG. 7E shows relative mRNA production in HCT116 cells co-transfected with a pool of three sgRNAs targeted to the EPCAM promoter with the indicated dCas9 fusions. Expression data are shown in comparison to cells transfected by dCas9 with no ED (Tukey-test, *P<0.05, ** P<0.01, (A,E) n=2 and (B,C,D) n−3 independent experiments; mean±SEM). FIG. 7F shows the positions of sgRNAs for each promoter.

FIGS. 8A-8E show variation in the repressive activity of various dCas9 FOG1[1-45] fusions at three promoters in two cell types. FIG. 8A shows expression of the HER2 promoter in HCT116 cells. FIG. 8B shows expression of the HER2 promoter in HEK293T cells. FIG. 8C shows expression of the EPCAM promoter in HCT116 cells. FIG. 8D shows expression of the MYC promoter in HCT116 cells. FIG. 8E shows expression of the MYC promoter in HEK293T cells. Although both FOG1[1-45]-dCas9 and dCas9-FOG1[1-45] showed repressive activity at the HER2 promoter in HEK293T cells (FIG. 8B), the most robust repression was again achieved with FOG1[1-45]-dCas9-FOG1[1-45] (Tukey test, *P<0.05; FIG. 8B). All dCas9-FOG1[1-45] fusions exhibited similar repressive activity at the EPCAM promoter in HCT116 cells (FIG. 8C). The C-terminal fusion protein dCas9-FOG1[1-45] showed a modest 1.4-fold repression and while other fusions resulted in 1.6-fold to 1.7-fold repression (Tukey test, *P<0.05; FIG. 8C). dCas9-FOG1[1-45] fusion proteins were able to repress MYC expression between 2-fold and 2.4-fold in HEK293T cells (Tukey test, **P<0.01; FIG. 8E), while repressive activity was not observed at the MYC promoter in HCT116 cells (FIG. 8D).

FIGS. 9A-9E show that dCpf1-epigenetic fusions did not repress HER2 gene expression. FIG. 9A shows a schematic of dCpf1-fusions with effector domains (ED). Catalytically-inactive AsCpf1 contained the nuclease-inactivating mutation D908A (dCpf1). FIG. 9A discloses “(GGS)₅” as SEQ ID NO: 75. FIG. 9B shows a UCSC genome browser graphic showing HER2 target regions of sgRNAs containing the 5′-NGG-3′ PAM required by dCas9, and crRNA target sites flanked by the 5′-NTTT-3′ PAM required by dCpf1. HCT116 ENCODE tracks for DNase Hypersensitivity (DNase HS) and H3K27ac binding are shown. FIG. 9C shows the abundance of HER2 mRNA that was measured after co-transfection of HCT116 cells with a pool of three crRNAs with the indicated dCpf1-ED fusions. No significant repression was observed compared to a dCpf1 with no ED. Negative control cells (“−”) were transfected with mCherry reporter plasmid instead of dCpf1. As a positive control, repression was assessed after co-transfection of dCas9 with no ED or FOG1[1-45]-dCas9-FOG1[1-45] and three sgRNAs (Tukey test, P<0.01, n=2 independent experiments; mean±SEM). FIG. 9D shows dCas9 and dCpf1 enrichments that were assessed by ChIP-qPCR at the HER2 promoter after transfection with a dCas9 or dCpf1 with no ED and the indicated sgRNA or crRNA. Statistical significance was analyzed by combining enrichments in the absence of a sgRNA or crRNA (n=2) and compared to dCas9 with sgRNA2 and sgRNA pool data (n=2) and with combined dCpf1/crRNA data (n=4) (Tukey test, P=0.001). ChIP assays using normal rabbit IgG were used as negative controls. FIG. 9E shows dCpf1 enrichments that were assayed after transfection with the indicated dCpf1 with no ED or the indicated fusion, and the three crRNAs (ns, not significant; n=2 independent experiments; mean±SEM). ChIP assays using normal rabbit IgG as negative controls are shown.

FIGS. 10A-10C show that combinations of epigenetic modifiers could achieve long-term gene repression. FIG. 10A shows a schematic of the experimental design for transient transfection assays with partial puromycin enrichment. FIG. 10B shows relative HER2 mRNA production in HCT116 cells co-transfected with a pool of three sgRNAs targeted to the HER2 gene promoter and combinations of N- or C-terminal DNMT3A-dCas9 fusions, KRAB-dCas9, and DNMT3L. FIG. 10C shows relative HER2 mRNA production using combinations of N-terminal DNMT3A-dCas9, KRAB-dCas9, DNMT3L, FOG1[1-45]-dCas9-FOG1[1-45], and Ezh2[FL]-dCas9. Expression data are shown in comparison to cells transfected by dCas9 with no ED. Statistical significance was analyzed for the transient effect by comparing dCas9 fusions to dCas9 without an effector domain after 4 days, while significance of persistent repression was calculated by comparing dCas9 fusions to dCas9 without ED after 14 days (Tukey test, *P<0.05, **P<0.01, n=2 independent experiments; mean±SEM).

### DETAILED DESCRIPTION OF THE INVENTION

### I. INTRODUCTION

Epigenome editing is an emerging tool to alter epigenetic marks at defined genomic loci (6). Precise DNA targeting was first accomplished with the design of programmable proteins based on zinc fingers (ZFs) and Transcription Activator-Like Effectors (TALEs) (7, 8). However, the field has been revolutionized by the discovery of the RNA-guided DNA-targeting platform CRISPR/Cas9 (clustered, regularly interspaced, short palindromic repeat/CRISPR-associated protein 9) (9, 10). dCas9 can be fused to heterologous effector domains to regulate transcription in a highly specific manner (13-15).

There has been considerable focus on dCas9-tethered epigenetic enzymes that alter DNA methylation. In particular, dCas9 fusions to DNMT3A/B or TET1/2 have been shown to target the deposition of 5-methylcytosine (5-mC) or the acquisition of 5-hydroxy-mC (5-hmC, considered to be the initial step in the removal of DNA methylation), respectively (16-21). Fewer studies have explored dCas9 fusions with enzymes affecting histone modifications. Gene activation has been explored using the histone acetyltransferase p300, histone demethylase LSD1 and a H3K4 methylase (22-24). Gene repression has been attempted using dCas9-KRAB fusions (15, 25). The Krüppel associated box (KRAB) domain recruits endogenous chromatin modifying complexes including the KAP1 co-repressor complex (26, 27) and the nucleosome remodeling and deacetylase (NuRD) complex (28) and thus has the potential to both trimethylate histone H3 on lysine 9 and to deacetylate histones. Catalytic domains from several other enzymes that catalyze H3K9me3 (such as G9A and SUV39H1) have been linked to either zinc finger or TALE DNA-binding domains, causing repression of the HER2 gene promoter (29). Although H3K27me3 is associated with repression, Ezh2 (the catalytic subunit of the Polycomb repressive complex 2 that causes deposition of H3K27me3) has not yet been studied as a fusion to a programmable DNA-binding domain. Importantly, in these previous studies, only changes in gene expression were used to assess the efficacy of the targeted epigenetic regulators. Few studies have monitored the changes in histone modification at the target site bound by the epigenetic regulator. However, such studies are essential to dissect the cause-and-effect relationship between histone modifications and transcriptional regulation.

The present invention is based, in part, on the development of novel fusion proteins comprising a catalytically-inactive Cas9 (dCas9) domain and an effector domain that imparts the ability of the fusion proteins to make epigenetic modifications at target chromatin sites. In particular, the inventors discovered that fusion proteins comprising dCas9 and either Krüppel-associated box (KRAB) or the N-terminal 45 amino acids of Friend of GATA1 (FOG1) were not only able to effect epigenetic modifications of chromatin, but were also particularly potent transcriptional repressors.

### II. DEFINITIONS

As used herein, the following terms have the meanings ascribed to them unless specified otherwise.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

The terms “about” and “approximately” as used herein shall generally mean an acceptable degree of error for the quantity measured given the nature or precision of the measurements. Typical, exemplary degrees of error are within 20 percent (%), preferably within 10%, and more preferably within 5% of a given value or range of values. Any reference to “about X” specifically indicates at least the values X, 0.95X, 0.96X, 0.97X, 0.98X, 0.99X, 1.01X, 1.02X, 1.03X, 1.04X, and 1.05X. Thus, “about X” is intended to teach and provide written description support for a claim limitation of, e.g., “0.98X.”

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic AcidRes. 19:5081 (1991); Ohtsuka et al., J. Biol. Chem. 260:2605-2608 (1985); and Rossolini et al., Mol. Cell. Probes 8:91-98 (1994)). The term nucleic acid is used interchangeably with gene, cDNA, and mRNA encoded by a gene.

The term “gene” means the segment of DNA involved in producing a polypeptide chain. It may include regions preceding and following the coding region (leader and trailer) as well as intervening sequences (introns) between individual coding segments (exons).

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds having a structure that is different from the general chemical structure of an amino acid, but that functions in a manner similar to a naturally occurring amino acid.

There are various known methods in the art that permit the incorporation of an unnatural amino acid derivative or analog into a polypeptide chain in a site-specific manner, see, e.g., WO 02/086075.

Amino acids may be referred to herein by either the commonly known three letter symbols or by the one-letter symbols recommended by the IUPAC-IUB Biochemical Nomenclature Commission. Nucleotides, likewise, may be referred to by their commonly accepted single-letter codes.

The term “conservatively modified variants” applies to both amino acid and nucleic acid sequences. With respect to particular nucleic acid sequences, “conservatively modified variants” refers to those nucleic acids that encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein that encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M)
(see, e.g., Creighton, Proteins, W. H. Freeman and Co., N.Y. (1984)).

In the present application, amino acid residues are numbered according to their relative positions from the left most residue, which is numbered 1, in an unmodified wild-type polypeptide sequence.

The terms “polypeptide,” “peptide,” and “protein” are used interchangeably herein to refer to a polymer of amino acid residues. All three terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins, wherein the amino acid residues are linked by covalent peptide bonds.

The term “expression vector” refers to a nucleic acid construct, generated recombinantly or synthetically, with a series of specified nucleic acid elements that permit transcription of a particular polynucleotide sequence (e.g., encoding a fusion protein of the present invention or a guide RNA molecule) in a host cell. An expression cassette may be part of a plasmid, viral genome, or nucleic acid fragment. Typically, an expression cassette includes a polynucleotide to be transcribed, operably linked to a promoter. Other elements that may be present in an expression cassette include those that enhance transcription (e.g., enhancers) and terminate transcription (e.g., terminators), as well as those that confer certain binding affinity or antigenicity to the recombinant protein produced from the expression cassette.

The term “enhancer of zeste homolog 2 (Ezh2)” refers to a histone-lysine-N-methyltransferase that is encoded by the EZH2 gene and participates, for example, in the methylation of lysine 27 of histone H3 (H3K27). Ezh2 methylation facilitates the formation of heterochromatin and subsequent suppression of gene expression. Ezh2 serves as the catalytic subunit of the Polycomb Repressive Complex 2 (PRC2), which plays important roles in embryonic development through the epigenetic regulation of genes that are involved with development and differentiation. Non-limiting examples of human Ezh2 mRNA sequences are set forth under NCBI Reference Sequence identifiers NM_004456.4→NP_004447.2 (transcript variant 1), NM_152998.2→NP_694543.1 (transcript variant 2), NM_001203247.1→NP_001190176.1 (transcript variant 3), NM_ NM_001203248.1→NP_001190177.1 (transcript variant 4), and NM_001203249.1→NP_001190178.1 (transcript variant 5). A non-limiting example of a human Ezh2 amino acid sequence is set forth under NCBI Reference Sequence identifier AAH10858.1 (SEQ ID NO:67). A non-limiting example of a mouse Ezh2 amino acid sequence is set forth under NCBI Reference Sequence identifier NP_031997.2.

The term “conserved cysteine-rich (CXC) domain” refers to a region near the C-terminal end of Ezh2, located N-terminal to the SET domain, that is coordinated by two clusters of three zinc ions. Mutations within the CXC domain are associated with a decrease in histone methyltransferase activity. As a non-limiting example, the CXC domain of Ezh2 in humans spans from about amino acid 503 to about amino acid 605 of the amino acid sequence set forth under NCBI Reference Sequence identifier AAH10858.1 (SEQ ID NO:67).

The term “Su(var)3-9, Enhancer-of-zeste and Trithorax (SET) domain” refers to a protein domain that is commonly present as part of a larger multidomain protein. In the context of the present invention, a SET domain is found near the C-terminal end of Ezh2, spanning the region from about amino acid 617 to about amino acid 738. The SET domain functions as the catalytic active site of Ezh2, and can play a role in determining substrate preference. For example, mutating the tyrosine at amino acid position 641 to phenylalanine has been shown to convey a preference for H3K27 trimethylation.

The term “embryonic ectoderm development (EED) binding domain” refers to a region near the N-terminal end of Ezh2, spanning the region from about amino acid 39 to about amino acid 67, that is required for histone methyltransferase activity. In particular, the EED binding domain is required for recognition of Ezh2 by the EED protein, which is part of the PRC2.

The term “Friend of GATA1 (FOG1)” refers to a zinc finger protein also known as ZFPM1 that is encoded by the ZFPM1 gene and is a cofactor of the GATA1 transcription factor. FOG1 is involved with recruiting the nucleosome remodeling and deacetylase (NuRD) complex to target sites, causing deacetylation, as well as methylation of lysine 27 of histone H3 via the recruitment of the PRC2. A non-limiting example of a human FOG1 mRNA sequence is set forth under NCBI Reference Sequence identifier NM_NM_153813.2→NP_722520.2. A non-limiting example of a human FOG1 amino acid sequence is set forth under NCBI Reference Sequence identifier AAN45858.1.

The term “euchromatic histone-lysine N-methyltransferase 2 (G9A)” refers to a histone methyltransferase that is also known as EHMT2 and is encoded by the EHMT2 gene in humans. G9A participates in the methylation of lysine 9 of histone H3 (H3K9), which is associated with the suppression of gene expression. Non-limiting examples of human G9A mRNA sequences are set forth under NCBI Reference Sequence identifiers NM_{'}001289413.1 4→NP_001276342.1 (transcript variant 1), NM_006709.4→NP_006700.3 (transcript variant 2), NM_025256.6→NP_079532.5 (transcript variant 3), and NM_001318833.1→NP_001305762.1 (transcript variant 4).

The term “histone-lysine N-methyltransferase SUV39H1 (SUV39H1)” refers to an enzyme that is encoded by the SUV39H1 gene in humans. SUV39H1 contains an N-terminal chromodomain and a C-terminal Su(var)3-9, Enhancer-of-zeste and Trithorax (SET) domain. SUV39H1 participates in the methylation of H3K9, which is associated with the suppression of gene expression. Non-limiting examples of human SUV39H1 mRNA sequences are set forth under NCBI Reference Sequence identifiers NM_001282166.1→NP_001269095.1 (transcript variant 1) and NM_003173.3→NP_003164.1 (transcript variant 2). A non-limiting example of an SUV39H1 amino acid sequence is set forth under NCBI Reference Sequence identifier NP_003164.1 (isoform 2).

The term “Krüppel-associated box (KRAB)” refers to a group of transcriptional repression domains that are present in about 400 human zinc finger protein-based transcription factors in humans. Typically, the KRAB domain contains about 75 amino acid residues, although the minimal repression module contains about 45 amino acid residues. Similar to FOG1, the KRAB domain functions by recruiting chromatin-modifying complexes to target sites. KRAB participates in the trimethylation of H3K9, which is achieved with the KRAB-associated protein 1 (KAP1) co-repressor complex. Over 10 independently coded KRAB domains have been identified that are functional suppressors of transcription. Non-limiting examples of human genes that encode KRAB zinc finger proteins include ZNF10, ZNF708, ZNF43, ZNF184, ZNF91, HPF4, HTF10 and HTF34. A non-limiting example of a human KRAB amino acid sequence is set forth under NCBI Reference Sequence identifier NP_056209.2.

The term “DNA (cytosine-5)-methyltransferase 3A (DNMT3A)” refers to a methyltransferase that is encoded by the DNMT3A gene in humans. DNMT3A catalyzes the methylation of CpG structures within DNA, in particular de novo DNA methylation, as opposed to maintenance methylation. DNA methylation by DNMT3A plays roles in cellular differentiation, embryonic development, transcriptional regulation (e.g., suppression of gene expression), heterochromatin formation, X-inactivation, imprinting, and the maintenance of genome stability. Non-limiting examples of human DNMT3A mRNA sequences are set forth under NCBI Reference Sequence identifiers NM_175629.2→NP_783328.1 (transcript variant 1), NM_153759.3 →NP_715640.2 (transcript variant 2), NM_022552.4→NP_072046.2 (transcript variant 3), NM_175630.1→NP_783329.1 (transcript variant 4), and NM_001320892.1→NP_001307821.1 (transcript variant 5).

The term “effector domain” refers to a protein, or a functional portion thereof, that modifies chromatin or a component thereof (e.g., a nucleic acid (e.g., DNA), a nucleotide, or a protein (e.g., a histone)). The chromatin or component thereof can be directly modified by the effector domain, or can be indirectly modified, e.g., by another protein that interacts with or is recruited by the effector domain. Non-limiting examples of modifications include methylation, demethylation, trimethylation, demethylation, acetylation, deacetylation, citrullination, and combinations thereof. In some embodiments, the effector domain produces two or more different modifications (e.g., deacetylation, followed by methylation). In such embodiments, the two or more different modifications can be achieved by the effector domain interacting with different additional proteins (i.e., the effector domain recruits or interacts with different proteins, each of which participates in or produces a different modification. As non-limiting examples, an effector domain can participate in the epigenetic modification of nucleotides, specific structures within nucleic acids (e.g., CpG structures), histones (e.g., histone H3), specific amino residues within a histone (e.g., lysine residues such as lysine 9 or lysine 27 of histone H3), or any combination thereof.

The terms “nuclear localization signal (NLS)” and “nuclear localization signal domain” refer to a peptide comprising an amino acid sequence that causes a protein (i.e., a protein to which the NLS is attached) to be imported into the nucleus of a cell. Typically, an NLS comprises one or more short sequences of positively charged amino acid residues (e.g., lysine, arginine). NLSs are commonly classified as being either classical or non-classical. Furthermore, classical NLSs are commonly classified as being either monopartite or bipartite, wherein bipartite NLSs contain two clusters of basic amino acid residues that are separated by a short peptide linker (e.g., a peptide of about 10 amino acids in length). A non-limiting example of a monopartite NLS is the Simian Vacuolating Virus 40 (SV40) NLS, having the sequence PKKKRKV (SEQ ID NO:68) or PKKKRKVG (SEQ ID NO:69). A non-limiting example of a bipartite NLS is KRPAATKKAGQAKKKK (SEQ ID NO:70). Classical NLSs are commonly recognized by the importin α class of nuclear import adaptor proteins, which are in turn recognized by importin β. Non-classical NLSs are typically recognized by importin β receptors without the involvement of importin α proteins.

The term “FLAG epitope tag” refers to a peptide having the sequence motif DYKDDDDK (SEQ ID NO:65). FLAG epitope tags can be used for protein purification (e.g., by affinity chromatography). In addition, by using antibodies that recognize the FLAG epitope, FLAG epitope tags can be used for the detection of proteins (i.e., when the protein or a complex comprising the protein contains the FLAG epitope tag), which is especially useful when no antibody specific for the protein of interest is readily available. In some instances, a FLAG epitope tag comprises a longer sequence, such as DYKDHDGDYKDHDIDYKDDDDK (SEQ ID NO:66).

The term “amino acid linker” refers to a contiguous sequence of amino acids that links one domain or portion of a fusion protein of the present invention to another. Amino acid linkers can contain natural amino acids, unnatural amino acids, or a combination thereof. In the context of the present invention, amino acids linkers commonly comprise a combination of glycine and serine amino acids. An amino acid linker can be of any length, and can contain any number of repeat units (e.g., repeat units comprising the sequence GGS (SEQ ID NO: 71)). Repeat units can be of any length.

The term “dCas9” refers to a Cas9 nuclease that contains one or more mutations that decrease or abolish the nuclease activity of Cas9, but leave the ability of Cas9 to function as an RNA-guided DNA-binding protein intact. As a non-limiting example, dCas9 can refer to a Cas9 nuclease that contains the two single amino acid mutations D10A and H840A, which render Cas9 catalytically inactive. The terms “catalytically inactive Cas9 domain” and “dCas9 domain” refer to a dCas9 protein, or a functional portion thereof (i.e., a portion of dCas9 that retains the ability of the protein to function as an RNA-guided DNA-binding protein), that recognizes and binds to a Cas9 recognition site as described herein.

### III. FUSION PROTEINS

In one aspect, the present invention provides a fusion protein comprising (1) a catalytically inactive Cas nuclease (e.g., catalytically inactive Cas9, or dCas9) domain and (2) an effector domain. In some embodiments, the effector domain is selected form the group consisting of enhancer of zeste homolog 2 (Ezh2), Friend of GATA1 (FOG1), histone H3 lysine 9 methyltransferase G9A (G9A), histone-lysine N-methyltransferase SUV39H1 (SUV39H1), Krüppel-associated box (KRAB), and DNA (cytosine-5)-methyltransferase 3A (DNMT3A). The fusion protein can contain 1, 2, 3, 4, 5, or 6 effector domains selected from the group consisting of Ezh2, FOG1, G9A, SUV39H1, KRAB, and DNMT3A. In particular embodiments, the effector domain is Ezh2 and/or FOG1. In some embodiments, the fusion protein comprises a DNMT3A effector domain and a full-length Ezh2 domain. The effector domain can comprise a full-length protein (e.g., full-length Ezh2, FOG1, G9A, SUV39H1, KRAB, or DNMT3A) or can comprise a functional portion or fragment of the full-length protein. In some embodiments, the effector domain comprises a catalytic domain of the full-length protein (e.g., a domain that is capable of producing an epigenetic modification (e.g., acetylation or methylation)).

The effector domain can be located either N-terminal or C-terminal to the catalytically inactive Cas nuclease (e.g., dCas9) domain. In some embodiments, the effector domain is located both N-terminal and C-terminal to the catalytically inactive Cas nuclease domain. In some embodiments, the fusion protein further comprises a nuclear localization signal (NLS) domain. In other embodiments, the fusion protein further comprises a FLAG epitope tag. In some embodiments, the fusion protein further comprises an amino acid linker. The fusion protein can comprise 1, 2, 3, 4, 5, or more NLS domains, FLAG epitope tags, and/or amino acid linkers, which can be present in any number of combinations. In some embodiments, the fusion protein comprises two NLS domains. In other embodiments, the fusion protein comprises a FLAG epitope tag. In particular embodiments, the fusion protein comprises two NLS domains and a FLAG epitope tag.

In some embodiments, the amino acid linker has about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acid residues. In other embodiments, the amino acid linker has at least about 50, 55, 60, 65, 70, 75, 80, 85, 90, 95, 100, or more amino acid residues. In some embodiments, the amino acid linker comprises one or more repeat units (e.g., 1, 2, 3, 4, 5, 6, 7, 8, 9, 10, 11, 12, 13, 14, 15, 16, 17, 18, 19, 20, or more repeat units). Each repeat unit can have, for example, 1, 2, 3, 4, 5, 6, 7, 8, 9, 10, or more amino acid residues. The amino acids can be natural amino acids, unnatural amino acids, or any combination thereof. In some embodiments, the amino acid linker comprises repeat units that have three amino acids (e.g., GGS (SEQ ID NO: 71)). In particular embodiments, the amino acid linker has the amino acid sequence (GGS)ₙ (SEQ ID NO: 71), wherein the subscript n is the number of repeat units. In some embodiments, the amino acid linker comprises the amino acid sequence of any one of SEQ ID NOS:71-80. In some instances, n is 5 (SEQ ID NO:75).

In some embodiments, the NLS domain, the FLAG epitope tag, and/or the amino acid linker are located N-terminal to the catalytically inactive Cas nuclease (e.g., dCas9) domain. In other embodiments, the NLS domain, the FLAG epitope, and/or the amino acid linker are located C-terminal to the catalytically inactive Cas nuclease domain. In some embodiments, the NLS domain, the FLAG epitope tag, and/or the amino acid linker are located both N-terminal to the catalytically inactive Cas nuclease domain and C-terminal to the catalytically inactive Cas nuclease domain. As non-limiting examples, amino acid linkers can be located between the catalytically inactive Cas nuclease domain and the effector domain, between two or more effector domains (i.e., when the fusion protein comprises a plurality of fusion domains), between the catalytically inactive Cas nuclease domain and the NLS domain, between the catalytically inactive Cas nuclease domain and the FLAG epitope tag, between the FLAG epitope tag and the NLS domain, or any combination thereof.

When the fusion protein comprises two or more effector domains, they can be all of the same type, they can each be different, or a combination thereof. The effector domains can be located N-terminal to the catalytically inactive Cas nuclease (e.g., dCas9) domain, C-terminal to the catalytically inactive Cas nuclease domain, or both N-terminal to the catalytically inactive Cas nuclease domain and C-terminal to the catalytically inactive Cas nuclease domain. In some embodiments, the fusion protein comprises 1, 2, 3, 4, 5, 6, 7, 8, 9, 10, or more effector domains that are N-terminal to the catalytically inactive Cas nuclease domain. In other embodiments, the fusion protein comprises 1, 2, 3, 4, 5, 6, 7, 8, 9, 10, or more effector domains that are C-terminal to the catalytically inactive Cas nuclease domain. In still other embodiments, the fusion protein comprises 1, 2, 3, 4, 5, 6, 7, 8, 9, 10, or more effector domains that are N-terminal to the catalytically inactive Cas nuclease domain and 1, 2, 3, 4, 5, 6, 7, 8, 9, 10, or more effector domains that are C-terminal to the catalytically inactive Cas nuclease domain. In some instances, the fusion protein comprises one effector domain that is N-terminal to the catalytically inactive Cas nuclease domain. In other instances, the fusion protein comprises one effector domain that is C-terminal to the catalytically inactive Cas nuclease domain. In some other instances, the fusion protein comprises one or more effector domains that are N-terminal to the catalytically inactive Cas nuclease domain the one or more effector domains that are C-terminal to the catalytically inactive Cas nuclease domain. In still other instances, the fusion protein comprises, 2, 3, or 4 effector domains that are N-terminal to the catalytically inactive Cas nuclease domain.

In some embodiments, the effector domain is KRAB and is located N-terminal to a dCas9 domain. In particular embodiments, the fusion protein comprises a single KRAB effector domain, and the KRAB effector domain is not located C-terminal to a dCas9 domain. In other embodiments, the effector domain is DNMT3A and is located N-terminal to a dCas9 domain. In particular embodiments, the fusion protein comprises a single DNMT3A effector domain, and the DNMT3A effector domain is not located C-terminal to a dCas9 domain.

In some embodiments, the fusion protein comprises an effector domain that comprises a functional portion of Ezh2. In some embodiments, the functional portion of Ezh2 comprises the Su(var)3-9, Enhancer-of-zeste and Trithorax (SET) domain. In particular embodiments, the functional portion of Ezh2 comprises the CXC and SET domains. In some embodiments, the functional portion of Ezh2 further comprises the embryonic ectoderm development (EED) binding domain. In particular embodiments, the functional portion of Ezh2 comprises the SET domain, the CXC domain, and the EED binding domain. In some instances, the fusion protein comprises an effector domain that comprises a full-length Ezh2 protein. As a non-limiting example, the full-length Ezh2 effector domain can comprise amino acids 1-746 of SEQ ID NO:1.

In some embodiments, the fusion protein comprises an effector domain that comprises a functional portion of FOG1. In some embodiments, the functional portion of FOG1 comprises the N-terminal 45 amino acids of a full-length FOG1 protein (e.g., the full-length FOG1 protein having the amino acid sequence set forth under NCBI Reference Sequence identifier AAN45858.1). As a non-limiting example, the functional portion of FOG1 can comprise the amino acid sequence set forth in SEQ ID NO:3 (FOG1[1-45]). In other embodiments, the fusion protein comprises an effector domain the comprises a full-length FOG1 protein (e.g., the full-length FOG1 protein having the amino acid sequence set forth under NCBI Reference Sequence identifier AAN45858.1). When the fusion protein comprises a plurality of FOG1 effector domains, they can all comprise a functional portion of FOG1, they can all comprise full-length FOG1, or a combination thereof. In some embodiments, the fusion protein comprises 1, 2, 3, 4, or more effector domains, wherein each effector domain comprises the amino acid sequence set forth in SEQ ID NO:3 (FOG1[1-45]). In some embodiments, the functional portion of FOG1 comprises the N-terminal 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 amino acids of a full-length FOG1 protein (e.g., the full-length FOG1 protein having the amino acid sequence set forth under NCBI Reference Sequence identifier AAN45858.1). In other embodiments, the functional portion of FOG1 comprises the first about 50, 100, 150, 200, 250, 300, 350, 400, 450, 500, 600, 700, 800, 900, 1,000, or more N-terminal amino acids of a full-length FOG1 protein (e.g., the full-length FOG1 protein having the amino acid sequence set forth under NCBI Reference Sequence identifier AAN45858.1).

In some embodiments, the fusion protein comprises an effector domain that comprises the amino acid sequence set forth in SEQ ID NO:3 (FOG1[1-45]). In some instances, the fusion protein further comprises an NLS domain that is located at the N-terminal end of the fusion protein. In some instances, the fusion protein further comprises an NLS domain that is located at the C-terminal end of the fusion protein. In particular embodiments, the fusion protein comprises a first NLS domain is located at the N-terminal end of the fusion protein, and a second NLS domain is located at the C-terminal end of the fusion protein. In some embodiments, the fusion protein further comprises a FLAG epitope tag that is located between the NLS domain that the N-terminal end of the protein and the N-terminal end of the catalytically inactive Cas nuclease (e.g., dCas9) domain. As a non-limiting example, the fusion protein can further comprise a FLAG epitope tag that is located between the first NLS domain and the N-terminal end of the catalytically inactive Cas nuclease domain.

In some embodiments, the FOG1 effector domain comprises 1, 2, 3, or 4 FOG1 effector domains that are located between the FLAG epitope tag and the N-terminal end of the catalytically inactive Cas nuclease (e.g., dCas9) domain. In some instances, each of the 1, 2, 3, or 4 FOG1 effector domains comprises the amino acid sequence set forth in SEQ ID NO:3 (FOG1[1-45]). In particular embodiments, the fusion protein further comprises one or more amino acid linkers. In some instances, the amino acid linker(s) comprise the amino acid sequence (GGS)ₙ, wherein n is 5 (SEQ ID NO:75). The amino acid linker(s) can be located between the FOG1 effector domain (e.g., 1, 2, 3, or 4 FOG1 effector domains) and the N-terminal end of the catalytically inactive Cas nuclease domain, between the C-terminal end of the catalytically inactive Cas nuclease domain and the second NLS domain, between adjacent FOG1 effector domains (i.e., when the fusion protein contains 2 or more FOG1 effector domains), between the FOG1 effector domain and the FLAG epitope tag, between the FLAG epitope tag and the first NLS domain, or a combination thereof.

In some embodiments, the FOG1 effector domain is located between the second NLS domain and the C-terminal end of the catalytically inactive Cas nuclease (e.g., dCas9) domain. In some instances, the FOG1 effector domain comprises the amino acid sequence set forth in SEQ ID NO:3 (FOG1[1-45]). In particular embodiments, the fusion protein further comprises one or more amino acid linkers. In some instances, the amino acid linker(s) comprise the amino acid sequence (GGS)ₙ, wherein n is 5 (SEQ ID NO:75). The amino acid linker(s) can be located between the FLAG epitope tag and the N-terminal end of the catalytically inactive Cas nuclease domain, between the C-terminal end of the catalytically inactive Cas nuclease domain and the FOG1 effector domain, between the FOG1 effector domain and the second NLS domain, between the FLAG epitope tag and the first NLS domain, or a combination thereof.

In some embodiments, a first FOG1 effector domain is located between the FLAG epitope tag and the N-terminal end of the catalytically inactive Cas nuclease (e.g., dCas9) domain, and a second FOG1 effector domain is located between the C-terminal end of the catalytically inactive Cas nuclease domain and the second NLS domain. In some instances, one or both FOG1 effector domains comprise the amino acid sequence set forth in SEQ ID NO:3 (FOG1[1-45]). In particular embodiments, the fusion protein further comprises one or more amino acid linkers. In some instances, the amino acid linker(s) comprise the amino acid sequence (GGS)ₙ, wherein n is 5 (SEQ ID NO:75). The amino acid linker(s) can be located between the first FOG1 effector domain and the N-terminal end of the catalytically inactive Cas nuclease domain, between the C-terminal end of the catalytically inactive Cas nuclease domain and the second FOG1 effector domain, between the first FOG1 effector domain and the FLAG epitope tag, between the second FOG1 effector domain and the second NLS domain, between the FLAG epitope tag and the first NLS domain, or a combination thereof.

In some embodiments, the fusion protein comprises the amino acid sequence of SEQ ID NO:9. In some embodiments, the fusion protein comprises the amino acid sequence of SEQ ID NO:10. In some embodiments, the fusion protein comprises the amino acid sequence of SEQ ID NO:9 or 10, and the position denoted by “Z” comprises the amino acid sequence of SEQ ID NO:1. In some embodiments, the fusion protein comprises the amino acid sequence of SEQ ID NO:9 or 10, and the position denoted by “Z” comprises the amino acid sequence of SEQ ID NO:2. In some embodiments, the fusion protein comprises the amino acid sequence of SEQ ID NO:9 or 10, and the position denoted by “Z” comprises the amino acid sequence of SEQ ID NO:3. In some embodiments, the fusion protein comprises the amino acid sequence of SEQ ID NO:9 or 10, and the position denoted by “Z” comprises the amino acid sequence of SEQ ID NO:4. In some embodiments, the fusion protein comprises the amino acid sequence of SEQ ID NO:9 or 10, and the position denoted by “Z” comprises the amino acid sequence of SEQ ID NO:5. In some embodiments, the fusion protein comprises the amino acid sequence of SEQ ID NO:9 or 10, and the position denoted by “Z” comprises the amino acid sequence of SEQ ID NO:6. In some embodiments, the fusion protein comprises the amino acid sequence of SEQ ID NO:9 or 10, and the position denoted by “Z” comprises the amino acid sequence of SEQ ID NO:7.

In some embodiments, the fusion protein comprises an amino acid sequence having at least about 90% (e.g., at least about 90%, 91%, 92%, 93%, 94%, 95%, 96%, 97%, 98%, or 99%) identity to any one of SEQ ID NOS:81-94. In some embodiments, the fusion protein comprises the amino acid sequence of any one of SEQ ID NOS:81-94.

### IV. CAS NUCLEASES

Fusion proteins of the present invention comprise a catalytically inactive Cas nuclease domain that comprises a catalytically inactive Cas nuclease (e.g., catalytically inactive Cas9, or dCas9) protein, or a fragment thereof, that has the ability to target a particular polynucleotide sequence (e.g., a Cas9 recognition site) within a target chromatin site. As a non-limiting example, a catalytically inactive variant of Cas9 can be used in which the Cas9 contains two single amino acid mutations (e.g., D10A, H840A) that abolish its nuclease activity, giving rise to an RNA-guided DNA-binding protein that lacks enzymatic activity (dCas9) (10). Typically, the catalytically inactive Cas nuclease domain will comprise a Cas nuclease, or a fragment thereof, that contains one or more mutations that abolish or decrease the ability of the Cas nuclease to cleave DNA, but allow the Cas nuclease to retain the ability to recognize a desired polynucleotide sequence.

Cas nucleases (e.g., Cas9) are part of what is known as the Clustered Regularly Interspaced Short Palindromic Repeats (CRISPR)/CRISPR-associated protein (Cas) nuclease system, which is an engineered nuclease system that is based on the adaptive immune response of many bacteria and archaea. Briefly, when a bacterium is invaded by a virus or plasmid, segments of the viral or plasmid DNA are converted into CRISPR RNAs (crRNA) by the “immune” response. The crRNA then associates with a type of RNA called tracrRNA to guide the Cas nuclease to a region that is homologous to the crRNA in the target DNA called a “protospacer.” In the case of catalytically active Cas nucleases, the DNA is cleaved by the Cas nuclease to generate blunt ends at double-strand break sites that are specified by a guide sequence contained within the crRNA transcript that is about 20 nucleotides in length. Depending on the particular Cas nuclease, both the crRNA and the tracrRNA may be required for site-specific DNA recognition and cleavage. This system has been modified such that the crRNA and tracrRNA, if needed, can be combined into one molecule (i.e., a “single guide RNA” or “sgRNA”), and the crRNA equivalent portion of the guide RNA can be engineered to guide the Cas (e.g., Cas9) nuclease to target any desired sequence (e.g., a nucleotide sequence within a target chromatin site).

Catalytically inactive variants of any number of Cas nucleases can be used in fusion proteins of the present invention. There are three main types of Cas nucleases (type I, type II, and type III), and 10 subtypes including 5 type I, 3 type II, and 2 type III proteins. Type II Cas nucleases include Cas1, Cas2, Csn2, Cas9, and Cpf1. A number of Cas nucleases will be known to one of skill in the art, for which catalytically inactive variants (e.g., mutants) thereof and homologs, fragments, derivatives, and combinations of the catalytically inactive variants find utility in fusion proteins of the present invention.

Non-limiting examples of additional Cas nucleases for which catalytically inactive variants find utility in fusion proteins of the present invention include Cas1, Cas1B, Cas2, Cas3, Cas4, Cas5, Cash, Cas7, Cas8, Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1. For each of these examples, one of skill in the art will be able to identify mutants in which catalytic ability is abolished or decreased, but polynucleotide sequence-targeting ability is retained.

Catalytically inactive variants of Cas nucleases can be derived from a variety of bacterial species including, but not limited to, Veillonella atypical, Fusobacterium nucleatum, Filifactor alocis, Solobacterium moorei, Coprococcus catus, Treponema denticola, Peptoniphilus duerdenii, Catenibacterium mitsuokai, Streptococcus mutans, Listeria innocua, Staphylococcus pseudintermedius, Acidaminococcus intestine, Olsenella uli, Oenococcus kitaharae, Bifidobacterium bifidum, Lactobacillus rhamnosus, Lactobacillus gasseri, Finegoldia magna, Mycoplasma mobile, Mycoplasma gallisepticum, Mycoplasma ovipneumoniae, Mycoplasma canis, Mycoplasma synoviae, Eubacterium rectale, Streptococcus thermophilus, Eubacterium dolichum, Lactobacillus coryniformis subsp. Torquens, Ilyobacter polytropus, Ruminococcus albus, Akkermansia muciniphila, Acidothermus cellulolyticus, Bifidobacterium longum, Bifidobacterium dentium, Corynebacterium diphtheria, Elusimicrobium minutum, Nitratifractor salsuginis, Sphaerochaeta globus, Fibrobacter succinogenes subsp. Succinogenes, Bacteroides fragilis, Capnocytophaga ochracea, Rhodopseudomonas palustris, Prevotella micans, Prevotella ruminicola, Flavobacterium columnare, Aminomonas paucivorans, Rhodospirillum rubrum, Candidatus Puniceispirillum marinum, Verminephrobacter eiseniae, Ralstonia syzygii, Dinoroseobacter shibae, Azospirillum, Nitrobacter hamburgensis, Bradyrhizobium, Wolinella succinogenes, Campylobacter jejuni subsp. Jejuni, Helicobacter mustelae, Bacillus cereus, Acidovorax ebreus, Clostridium perfringens, Parvibaculum lavamentivorans, Roseburia intestinalis, Neisseria meningitidis, Pasteurella multocida subsp. Multocida, Sutterella wadsworthensis, proteobacterium, Legionella pneumophila, Parasutterella excrementihominis, Wolinella succinogenes, and Francisella novicida. For each of these examples, one of skill in the art will be able to clone nucleases and subsequently identify mutants in which catalytic ability is abolished or decreased, but polynucleotide sequence-targeting ability is retained.

“Cas9” refers to a particular type II Cas nuclease that is an RNA-guided double-stranded DNA-binding nuclease protein. Catalytically active Cas9 nuclease has two functional domains, e.g., RuvC and HNH, that cut different DNA strands. Cas9 requires two RNA molecules (e.g., a crRNA and a tracrRNA), or alternatively, a single guide RNA (sgRNA) that comprises a crRNA and a tracrRNA. Cas9 utilizes a G-rich protospacer-adjacent motif (PAM) that is 3′ of the guide RNA targeting sequence and creates double-strand cuts having blunt ends. As non-limiting examples, the amino acid sequence of the Streptococcus pyogenes wild-type Cas9 polypeptide is set forth, e.g., in NBCI Ref. Seq. No. NP_269215 and the amino acid sequence of Streptococcus thermophilus wild-type Cas9 polypeptide is set forth, e.g., in NBCI Ref. Seq. No. WP_011681470.

The fusion proteins of the present invention are typically guided to a target site (e.g., a target chromatin site containing a Cas recognition site (e.g., Cas9 recognition site)) by a guide RNA (gRNA) (e.g., a single guide RNA (sgRNA)). The gRNAs for use in the methods of the present invention typically include a crRNA sequence that is complementary to a target nucleic acid sequence and may include a scaffold sequence (e.g., tracrRNA) that interacts with a Cas nuclease variant (e.g., dCas9) or fragment thereof, depending on the particular nuclease being used.

The gRNA molecule can comprise any nucleic acid sequence, so long as the sequence has sufficient complementarity to the intended target polynucleotide sequence (e.g., target DNA sequence at or near the target chromatin site) to permit hybridization with the target sequence and direct sequence-specific binding of a catalytically inactive Cas nuclease domain (and thus the fusion protein) to the target sequence. The gRNA molecule typically recognizes a PAM sequence that is near or adjacent to the target sequence. The target DNA site may be located immediately 5′ of a PAM sequence, the PAM sequence being specific to the particular bacterial species of the catalytically inactive Cas nuclease being used. Non-limiting examples of PAM sequences include NGG (Streptococcus pyogenes), NNNNGATT (Neisseria meningitidis), NNAGAA (Streptococcus thermophilus), NAAAAC (Treponema denticola). The PAM sequence can be NGG, wherein N is any nucleotide, NRG, wherein N is any nucleotide and R is a purine, or NNGRR, wherein N is any nucleotide and R is a purine. For Cas nucleases derived from S. pyogenes, the target sequence should immediately precede (i.e., be located 5′ of) a 5′NGG PAM.

In some embodiments, the degree of complementarity between a guide sequence of the gRNA (i.e., the crRNA sequence) and its corresponding target sequence is about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more. In some embodiments, a crRNA sequence is about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more nucleotides in length. In some embodiments, the crRNA sequence is about 20, 21, 22, 23, 24, or 25 nucleotides in length.

In some embodiments, the length of the gRNA molecule is about 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, or more nucleotides in length. In some instances, the length of the gRNA is about 100 nucleotides in length.

Non-limiting examples of algorithms for determining sequence complementarity include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies, ELAND, SOAP, and Maq.

### V. PRODUCTION, EXPRESSION, AND PURIFICATION OF FUSION PROTEINS

### A. General Recombinant Technology

Basic texts disclosing general methods and techniques in the field of recombinant genetics include Sambrook and Russell, Molecular Cloning, A Laboratory Manual (3rd ed. 2001); Kriegler, Gene Transfer and Expression: A Laboratory Manual (1990); and Ausubel et al., eds., Current Protocols in Molecular Biology (1994).

For nucleic acids, sizes are given in either kilobases (kb) or base pairs (bp). These are estimates derived from agarose or acrylamide gel electrophoresis, from sequenced nucleic acids, or from published DNA sequences. For proteins, sizes are given in kilodaltons (kDa) or amino acid residue numbers. Proteins sizes are estimated from gel electrophoresis, from sequenced proteins, from derived amino acid sequences, or from published protein sequences.

Oligonucleotides that are not commercially available can be chemically synthesized, e.g., according to the solid phase phosphoramidite triester method first described by Beaucage & Caruthers, Tetrahedron Lett. 22: 1859-1862 (1981), using an automated synthesizer, as described in Van Devanter et. al., Nucleic Acids Res. 12: 6159-6168 (1984). Purification of oligonucleotides is performed using any art-recognized strategy, e.g., native acrylamide gel electrophoresis or anion-exchange HPLC as described in Pearson & Reanier, J. Chrom. 255: 137-149 (1983).

The sequence of a protein domain or gene of interest, such as a Cas nuclease (e.g., Cas9) domain or an effector domain, can be verified after cloning or subcloning using, e.g., the chain termination method for sequencing double-stranded templates of Wallace et al., Gene 16: 21-26 (1981).

A large number of possible tags may be used for practicing the present invention. Non-limiting examples include: biotin (small molecule); StrepTag (StrepII) (8 a.a.); SBP (38 a.a.); biotin carboxyl carrier protein or BCCP (100 a.a.); epitope tags such as FLAG (8 a.a.), 3× FLAG (22 a.a.), and myc (22 a.a.); S-tag (Novagen) (15 a.a.); Xpress (Invitrogen) (25 a.a.); eXact (Bio-Rad) (75 a.a.); HA (9 a.a.); VSV-G (11 a.a.); Protein A/G (280 a.a.); HIS (6-10 a.a.) (SEQ ID NO: 96); glutathione s-transferase or GST (218 a.a.); maltose binding protein or MBP (396 a.a.); CBP (28 a.a.); CYD (5 a.a.); HPC (12 a.a.); CBD intein-chitin binding domain (51 a.a.); Trx (Invitrogen) (109 a.a.); NorpA (5 a.a.); and NusA (495 a.a.).

### B. Coding Sequence for a Protein of Interest

In another aspect, the present invention provides nucleic acids that comprise a polynucleotide sequence encoding a fusion protein of the present invention. The rapid progress in the studies of human genome has made possible a cloning approach where a human DNA sequence database can be searched for any gene segment that has a certain percentage of sequence homology to a known nucleotide sequence, such as one encoding a previously Cas nuclease (e.g., Cas9) or an effector domain protein described herein. Any DNA sequence so identified can be subsequently obtained by chemical synthesis and/or a polymerase chain reaction (PCR) technique such as overlap extension method. For a short sequence, completely de novo synthesis may be sufficient; whereas further isolation of full length coding sequence from a human cDNA or genomic library using a synthetic probe may be necessary to obtain a larger gene.

Alternatively, a nucleic acid sequence can be isolated from a cDNA or genomic DNA library (e.g., human or rodent cDNA or genomic DNA library) using standard cloning techniques such as polymerase chain reaction (PCR), where homology-based primers can often be derived from a known nucleic acid sequence. Most commonly used techniques for this purpose are described in standard texts, e.g., Sambrook and Russell, supra.

cDNA libraries may be commercially available or can be constructed. The general methods of isolating mRNA, making cDNA by reverse transcription, ligating cDNA into a recombinant vector, transfecting into a recombinant host for propagation, screening, and cloning are well known (see, e.g., Gubler and Hoffman, Gene, 25: 263-269 (1983); Ausubel et al., supra). Upon obtaining an amplified segment of nucleotide sequence by PCR, the segment can be further used as a probe to isolate the full length polynucleotide sequence encoding the protein of interest from the cDNA library. A general description of appropriate procedures can be found in Sambrook and Russell, supra.

A similar procedure can be followed to obtain a full-length sequence encoding a protein of interest from a human genomic library. Human genomic libraries are commercially available or can be constructed according to various art-recognized methods. In general, to construct a genomic library, the DNA is first extracted from a tissue where a protein of interest is likely found. The DNA is then either mechanically sheared or enzymatically digested to yield fragments of about 12-20 kb in length. The fragments are subsequently separated by gradient centrifugation from polynucleotide fragments of undesired sizes and are inserted in bacteriophage λ vectors. These vectors and phages are packaged in vitro. Recombinant phages are analyzed by plaque hybridization as described in Benton and Davis, Science, 196: 180-182 (1977). Colony hybridization is carried out as described by Grunstein et al., Proc. Natl. Acad. Sci. USA, 72: 3961-3965 (1975).

Based on sequence homology, degenerate oligonucleotides can be designed as primer sets and PCR can be performed under suitable conditions (see, e.g., White et al., PCR Protocols: Current Methods and Applications, 1993; Griffin and Griffin, PCR Technology, CRC Press Inc. 1994) to amplify a segment of nucleotide sequence from a cDNA or genomic library. Using the amplified segment as a probe, the full-length nucleic acid encoding a protein of interest is obtained.

Upon acquiring a nucleic acid sequence encoding a protein of interest, such as a Cas nuclease (e.g., Cas9) or an effector domain protein, the coding sequence can be further modified by a number of well-known techniques such as restriction endonuclease digestion, PCR, and PCR-related methods to generate coding sequences, including mutants and variants derived from the wild-type protein. The polynucleotide sequence encoding the desired polypeptide can then be subcloned into a vector, for instance, an expression vector, so that a recombinant polypeptide can be produced from the resulting construct. Further modifications to the coding sequence, e.g., nucleotide substitutions, may be subsequently made to alter the characteristics of the polypeptide.

A variety of mutation-generating protocols are established and described in the art, and can be readily used to modify a polynucleotide sequence encoding a protein of interest. See, e.g., Zhang et al., Proc. Natl. Acad. Sci. USA, 94: 4504-4509 (1997); and Stemmer, Nature, 370: 389-391 (1994). The procedures can be used separately or in combination to produce variants of a set of nucleic acids, and hence variants of encoded polypeptides. Kits for mutagenesis, library construction, and other diversity-generating methods are commercially available.

Mutational methods of generating diversity include, for example, site-directed mutagenesis (Botstein and Shortle, Science, 229: 1193-1201 (1985)), mutagenesis using uracil-containing templates (Kunkel, Proc. Natl. Acad. Sci. USA, 82: 488-492 (1985)), oligonucleotide-directed mutagenesis (Zoller and Smith, Nucl. Acids Res., 10: 6487-6500 (1982)), phosphorothioate-modified DNA mutagenesis (Taylor et al., Nucl. Acids Res., 13: 8749-8764 and 8765-8787 (1985)), and mutagenesis using gapped duplex DNA (Kramer et al., Nucl. Acids Res., 12: 9441-9456 (1984)).

Other possible methods for generating mutations include point mismatch repair (Kramer et al., Cell, 38: 879-887 (1984)), mutagenesis using repair-deficient host strains (Carter et al., Nucl. Acids Res., 13: 4431-4443 (1985)), deletion mutagenesis (Eghtedarzadeh and Henikoff, Nucl. Acids Res., 14: 5115 (1986)), restriction-selection and restriction-purification (Wells et al., Phil. Trans. R. Soc. Lond. A, 317: 415-423 (1986)), mutagenesis by total gene synthesis (Nambiar et al., Science, 223: 1299-1301 (1984)), double-strand break repair (Mandecki, Proc. Natl. Acad. Sci. USA, 83: 7177-7181 (1986)), mutagenesis by polynucleotide chain termination methods (U.S. Pat. No. 5,965,408), and error-prone PCR (Leung et al., Biotechniques, 1: 11-15 (1989)).

C. Modification of Nucleic Acids for Preferred Codon Usage in a Host Organism

The nucleic acid comprising a polynucleotide sequence encoding a protein of interest, e.g., a fusion protein of the present invention or a portion thereof (e.g., a Cas nuclease domain, effector domain), can be further altered to coincide with the preferred codon usage of a particular host. For example, the preferred codon usage of one strain of bacterial cells can be used to derive a polynucleotide that encodes a recombinant polypeptide of the invention and includes the codons favored by this strain. The frequency of preferred codon usage exhibited by a host cell can be calculated by averaging frequency of preferred codon usage in a large number of genes expressed by the host cell (e.g., calculation service is available from web site of the Kazusa DNA Research Institute, Japan). This analysis is preferably limited to genes that are highly expressed by the host cell.

At the completion of modification, the coding sequences are verified by sequencing and are then subcloned into an appropriate expression vector for recombinant production of a protein of interest, such as a fusion protein comprising a Cas nuclease domain or a variant thereof and an effector domain or a variant thereof.

Following verification of the coding sequence, a fusion protein of the present invention can be produced using routine techniques in the field of recombinant genetics, relying on the polynucleotide sequences encoding the polypeptide disclosed herein.

### D. Expression Systems

To obtain high level expression of a nucleic acid encoding a fusion protein of this invention, one typically subclones a polynucleotide encoding the protein of interest in the correct reading frame into an expression vector (e.g., an expression vector of the present invention the comprises a nucleic acid of the present invention) that contains a strong promoter to direct transcription, a transcription/translation terminator and a ribosome binding site for translational initiation. Suitable bacterial promoters are well known in the art and described, e.g., in Sambrook and Russell, supra, and Ausubel et al., supra. Bacterial expression systems for expressing the polypeptide are available in, e.g., E. coli, Bacillus sp., Salmonella, and Caulobacter. Kits for such expression systems are commercially available. Eukaryotic expression systems for mammalian cells (including human cells), yeast, and insect cells are well known in the art and are also commercially available. In one embodiment, the eukaryotic expression vector is an adenoviral vector, an adeno-associated vector, or a retroviral vector.

The promoter used to direct expression of a heterologous nucleic acid depends on the particular application. The promoter is optionally positioned about the same distance from the heterologous transcription start site as it is from the transcription start site in its natural setting. As is known in the art, however, some variation in this distance can be accommodated without loss of promoter function.

In another aspect, the present invention provides host cells that have been transfected by expression vectors of the present invention (i.e., expression vectors comprising nucleic acids that comprise nucleotide sequences encoding fusion proteins of the present invention). The compositions and methods of the present invention can be used for producing epigenetic modifications in the genome of any host cell of interest. The host cell can be a cell from any organism, e.g., a bacterial cell, an archaeal cell, a cell of a single-cell eukaryotic organism, a plant cell (e.g., a rice cell, a wheat cell, a tomato cell, an Arabidopsis thaliana cell, a Zea mays cell and the like), an algal cell (e.g., Botryococcus braunii, Chlamydomonas reinhardtii, Nannochloropsis gaditana, Chlorella pyrenoidosa, Sargassum patens C. Agardh, and the like), a fungal cell (e.g., yeast cell, etc.), an animal cell, a cell from an invertebrate animal (e.g., fruit fly, cnidarian, echinoderm, nematode, etc.), a cell from a vertebrate animal (e.g., fish, amphibian, reptile, bird, mammal, etc.), a cell from a mammal, a cell from a human, a cell from a healthy human, a cell from a human patient, a cell from a cancer patient, etc. In some cases, the host cell treated by the method disclosed herein can be transplanted to a subject (e.g., patient). For instance, the host cell in which the epigenetic modification is made can be derived from the subject to be treated (e.g., patient).

Epigenetic modifications by fusion proteins of the present invention can be made in any cell of interest, such as a stem cell, e.g., embryonic stem cell, induced pluripotent stem cell, adult stem cell, e.g., mesenchymal stem cell, neural stem cell, hematopoietic stem cell, organ stem cell, a progenitor cell, a somatic cell, e.g., fibroblast, hepatocyte, heart cell, liver cell, pancreatic cell, muscle cell, skin cell, blood cell, neural cell, immune cell, and any other cell of the body, e.g., human body. The cells can be primary cells or primary cell cultures derived from a subject, e.g., an animal subject or a human subject, and allowed to grow in vitro for a limited number of passages. In some embodiments, epigenetic modifications are made in cells that are disease cells or derived from a subject with a disease. For instance, the cells can be cancer or tumor cells. The cells can also be immortalized cells (e.g., cell lines), for instance, from a cancer cell line.

Depending on the host cell and expression system used, the expression vector (e.g., for expression of a fusion protein of the present invention and/or a gRNA molecule) may contain transcription and translation control elements, including promoters, transcription enhancers, transcription terminators, and the like. Useful promoters can be derived from viruses, or any organism, e.g., prokaryotic or eukaryotic organisms. Promoters may also be inducible (i.e., capable of responding to environmental factors and/or external stimuli that can be artificially controlled). For expressing fusion proteins of the present invention, non-limiting examples of promoters that find utility in expression vectors of the present invention include RNA polymerase II promoters (e.g., pGAL7 and pTEF1), RNA polymerase III promoters (e.g., RPR-tetO, SNR52, and tRNA-tyr), the SV40 early promoter, mouse mammary tumor virus long terminal repeat (LTR) promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, a human U6 small nuclear promoter (U6), an enhanced U6 promoter, a human H1 promoter (H1), etc. Suitable terminators for use in fusion protein-expressing vectors of the present invention include, but are not limited to SNR52 and RPR terminator sequences, which can be used with transcripts created under the control of a RNA polymerase III promoter. Additionally, various primer binding sites may be incorporated into a vector to facilitate vector cloning, sequencing, genotyping, and the like. Other suitable promoter, enhancer, terminator, and primer binding sequences will readily be known to one of skill in the art.

The particular expression vector used to transport the genetic information into the cell is not particularly critical. Any of the conventional vectors used for expression in eukaryotic or prokaryotic cells may be used. Standard bacterial expression vectors include plasmids such as pBR322 based plasmids, pSKF, pET23D, and fusion expression systems such as GST and LacZ. Epitope tags can also be added to recombinant proteins to provide convenient methods of isolation, e.g., c-myc.

Expression vectors containing regulatory elements from eukaryotic viruses are typically used in eukaryotic expression vectors, e.g., SV40 vectors, papilloma virus vectors, and vectors derived from Epstein-Barr virus. Other exemplary eukaryotic vectors include pMSG, pAV009/A⁺, pMTO10/A⁺, pMAMneo-5, baculovirus pDSVE, and any other vector allowing expression of proteins under the direction of the SV40 early promoter, SV40 later promoter, metallothionein promoter, murine mammary tumor virus promoter, Rous sarcoma virus promoter, polyhedrin promoter, or other promoters shown effective for expression in eukaryotic cells.

Some expression systems have markers that provide gene amplification such as thymidine kinase, hygromycin B phosphotransferase, and dihydrofolate reductase. Alternatively, high yield expression systems not involving gene amplification are also suitable, such as a baculovirus vector in insect cells, with a polynucleotide sequence encoding the protein of interest under the direction of the polyhedrin promoter or other strong baculovirus promoters.

The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of eukaryotic sequences. The particular antibiotic resistance gene chosen is not critical, any of the many resistance genes known in the art are suitable. The prokaryotic sequences are optionally chosen such that they do not interfere with the replication of the DNA in eukaryotic cells, if necessary. Similar to antibiotic resistance selection markers, metabolic selection markers based on known metabolic pathways may also be used as a means for selecting transfected host cells.

When periplasmic expression of a fusion protein of the present invention is desired, the expression vector further comprises a sequence encoding a secretion signal, such as the E. coli OppA (Periplasmic Oligopeptide Binding Protein) secretion signal or a modified version thereof, which is directly connected to 5′ of the coding sequence of the protein to be expressed. This signal sequence directs the recombinant protein produced in cytoplasm through the cell membrane into the periplasmic space. The expression vector may further comprise a coding sequence for signal peptidase 1, which is capable of enzymatically cleaving the signal sequence when the recombinant protein is entering the periplasmic space. More detailed description for periplasmic production of a recombinant protein can be found in, e.g., Gray et al., Gene 39: 247-254 (1985), U.S. Pat. Nos. 6,160,089 and 6,436,674.

A person skilled in the art will recognize that various conservative substitutions can be made to any wild-type or mutant/variant protein to produce a fusion protein of the present invention. Moreover, modifications of a polynucleotide coding sequence may also be made to accommodate preferred codon usage in a particular expression host without altering the resulting amino acid sequence.

### E. Transfection Methods

Standard transfection methods are used to produce bacterial, mammalian, yeast, insect, or plant cell lines that express large quantities of a recombinant fusion protein of this invention, which are then purified using standard techniques (see, e.g., Colley et al., J. Biol. Chem. 264: 17619-17622 (1989); Guide to Protein Purification, in Methods in Enzymology, vol. 182 (Deutscher, ed., 1990)). Transformation of eukaryotic and prokaryotic cells are performed according to standard techniques (see, e.g., Morrison, J. Bact. 132: 349-351 (1977); Clark-Curtiss & Curtiss, Methods in Enzymology 101: 347-362 (Wu et al., eds, 1983).

Any of the well-known procedures for introducing foreign nucleotide sequences into host cells may be used. These include the use of calcium phosphate transfection, polybrene, protoplast fusion, electroporation, liposomes, microinjection, plasma vectors, viral vectors and any of the other well-known methods for introducing cloned genomic DNA, cDNA, synthetic DNA, or other foreign genetic material into a host cell (see, e.g., Sambrook and Russell, supra). It is only necessary that the particular genetic engineering procedure used be capable of successfully introducing at least one gene into the host cell capable of expressing the fusion protein of this invention.

### F. Purification of Recombinantly Produced Fusion Proteins

Once the expression of a recombinant fusion protein of the present invention in transfected host cells is confirmed, e.g., via an immunoassay such as Western blotting assay, the host cells are then cultured in an appropriate scale for the purpose of purifying the recombinant polypeptide.

1. Purification of Recombinantly Produced Polypeptides from Bacteria

When the fusion proteins of the present invention are produced recombinantly by transformed bacteria in large amounts, typically after promoter induction, although expression can be constitutive, the polypeptides may form insoluble aggregates. There are several protocols that are suitable for purification of protein inclusion bodies. For example, purification of aggregate proteins (hereinafter referred to as inclusion bodies) typically involves the extraction, separation and/or purification of inclusion bodies by disruption of bacterial cells, e.g., by incubation in a buffer of about 100-150 μg/ml lysozyme and 0.1% Nonidet P40, a non-ionic detergent. The cell suspension can be ground using a Polytron grinder (Brinkman Instruments, Westbury, N.Y.). Alternatively, the cells can be sonicated on ice. Additional methods of lysing bacteria are described in Ausubel et al. and Sambrook and Russell, both supra, and will be apparent to those of skill in the art.

The cell suspension is generally centrifuged and the pellet containing the inclusion bodies resuspended in buffer which does not dissolve but washes the inclusion bodies, e.g., 20 mM Tris-HCl (pH 7.2), 1 mM EDTA, 150 mM NaCl and 2% Triton-X 100, a non-ionic detergent. It may be necessary to repeat the wash step to remove as much cellular debris as possible. The remaining pellet of inclusion bodies may be resuspended in an appropriate buffer (e.g., 20 mM sodium phosphate, pH 6.8, 150 mM NaCl). Other appropriate buffers will be apparent to those of skill in the art.

Following the washing step, the inclusion bodies are solubilized by the addition of a solvent that is both a strong hydrogen acceptor and a strong hydrogen donor (or a combination of solvents each having one of these properties). The proteins that formed the inclusion bodies may then be renatured by dilution or dialysis with a compatible buffer. Suitable solvents include, but are not limited to, urea (from about 4 M to about 8 M), formamide (at least about 80%, volume/volume basis), and guanidine hydrochloride (from about 4 M to about 8 M). Some solvents that are capable of solubilizing aggregate-forming proteins, such as SDS (sodium dodecyl sulfate) and 70% formic acid, may be inappropriate for use in this procedure due to the possibility of irreversible denaturation of the proteins, accompanied by a lack of immunogenicity and/or activity. Although guanidine hydrochloride and similar agents are denaturants, this denaturation is not irreversible and renaturation may occur upon removal (by dialysis, for example) or dilution of the denaturant, allowing re-formation of the immunologically and/or biologically active protein of interest. After solubilization, the protein can be separated from other bacterial proteins by standard separation techniques. For further description of purifying recombinant polypeptides from bacterial inclusion body, see, e.g., Patra et al., Protein Expression and Purification 18: 182-190 (2000).

Alternatively, it is possible to purify recombinant polypeptides from bacterial periplasm. Where the recombinant protein is exported into the periplasm of the bacteria, the periplasmic fraction of the bacteria can be isolated by cold osmotic shock in addition to other methods known to those of skill in the art (see e.g., Ausubel et al., supra). To isolate recombinant proteins from the periplasm, the bacterial cells are centrifuged to form a pellet. The pellet is resuspended in a buffer containing 20% sucrose. To lyse the cells, the bacteria are centrifuged and the pellet is resuspended in ice-cold 5 mM MgSO₄ and kept in an ice bath for approximately 10 minutes. The cell suspension is centrifuged and the supernatant decanted and saved. The recombinant proteins present in the supernatant can be separated from the host proteins by standard separation techniques well known to those of skill in the art.

2. Standard Protein Separation Techniques for Purification

When a recombinant polypeptide of the present invention, e.g., a fusion protein of the present invention is expressed in host cells (such as human cells) in a soluble form, its purification can follow the standard protein purification procedure described below. This standard purification procedure is also suitable for purifying fusion proteins obtained from chemical synthesis.

i. Solubility Fractionation

Often as an initial step, and if the protein mixture is complex, an initial salt fractionation can separate many of the unwanted host cell proteins (or proteins derived from the cell culture media) from the recombinant protein of interest, e.g., a fusion protein of the present invention. The preferred salt is ammonium sulfate. Ammonium sulfate precipitates proteins by effectively reducing the amount of water in the protein mixture. Proteins then precipitate on the basis of their solubility. The more hydrophobic a protein is, the more likely it is to precipitate at lower ammonium sulfate concentrations. A typical protocol is to add saturated ammonium sulfate to a protein solution so that the resultant ammonium sulfate concentration is between 20-30%. This will precipitate the most hydrophobic proteins. The precipitate is discarded (unless the protein of interest is hydrophobic) and ammonium sulfate is added to the supernatant to a concentration known to precipitate the protein of interest. The precipitate is then solubilized in buffer and the excess salt removed if necessary, through either dialysis or diafiltration. Other methods that rely on solubility of proteins, such as cold ethanol precipitation, are well known to those of skill in the art and can be used to fractionate complex protein mixtures.

ii. Size Differential Filtration

Based on a calculated molecular weight, a protein of greater and lesser size can be isolated using ultrafiltration through membranes of different pore sizes (for example, Amicon or Millipore membranes). As a first step, the protein mixture is ultrafiltered through a membrane with a pore size that has a lower molecular weight cut-off than the molecular weight of a protein of interest, e.g., a fusion protein of the present invention. The retentate of the ultrafiltration is then ultrafiltered against a membrane with a molecular cut off greater than the molecular weight of the protein of interest. The recombinant protein will pass through the membrane into the filtrate. The filtrate can then be chromatographed as described below.

iii. Column Chromatography

The proteins of interest (such as a fusion protein of the present invention) can also be separated from other proteins on the basis of their size, net surface charge, hydrophobicity, or affinity for ligands, such as amylose. In addition, antibodies raised against a segment of the protein of interest can be conjugated to column matrices and the target fusion protein can therefore be immunopurified. All of these methods are well known in the art.

It will be apparent to one of skill that chromatographic techniques can be performed at any scale and using equipment from many different manufacturers (e.g., Pharmacia Biotech).

### VI. METHODS FOR EPIGENETIC MODIFICATION

In another aspect, the present invention provides a method for producing an epigenetic modification of a target chromatin site. A nucleic acid component of chromatin (e.g., DNA) and/or a protein component of chromatin (e.g., a histone protein such as histone H3) present at the target site can be modified. In some embodiments, the target chromatin site comprises a Cas nuclease recognition site (e.g., a Cas9 recognition site). In some embodiments, the target chromatin site comprises a polynucleotide sequence that is recognized by a guide RNA (gRNA) molecule. In some embodiments, the method comprises contacting the target chromatin site with a fusion protein provided herein.

The term “epigenetic modification” refers to a change in genetic information that does not arise from a change in a nucleotide sequence (e.g., a DNA sequence). Typically, epigenetic modifications, such as those than can be produced by fusion proteins and other compositions of the present invention, affect the expression or activity of a target chromatin site (e.g., the expression or activity of a gene), although an epigenetic modification can be any modification of genetic material (e.g., chromatin or a component thereof) that does not arise from a nucleotide sequence change but produces a change in a phenotype (e.g., of an organism comprising the target chromatin site). In the context of the present invention, epigenetic modifications typically comprise modifications to a nucleic acid (e.g., DNA) or a protein (e.g., a histone)). Such modifications typically comprise methylation, dimethylation, trim ethylation, demethylation, acetylation, deacetylation, citrullination, or a combination thereof. Epigenetic modifications can either decrease or increase the expression or activity of a target chromatin site (e.g., gene expression or activity). Epigenetic modifications, and the resulting effects (e.g., changes in gene expression or phenotype), can be either transient or persistent. The choice of effector domain can be used to determine whether a transient or persistent epigenetic modification and/or resulting effect is produced. As a non-limiting example, a combination of a DNMT3A effector domain and a full-length Ezh2 domain can be used to achieve a persistent effect (e.g., gene silencing).

“Chromatin” refers to the macromolecular complex typically found in cells that comprises nucleic acids (e.g., DNA, RNA) and proteins (e.g., histones). Chromatin performs several functions, including packaging DNA into more compact forms, controlling DNA replication and gene expression (e.g., transcriptional regulation), and protecting against DNA damage. In eukaryotic cells, nucleosomes, which comprise DNA wrapped around histone proteins and are separated by relatively short sections of linker DNA, form the fundamental repeat unit of chromatin. Furthermore, multiple histones can wrap into a 30 nm fiber structure. The 30 nm fibers can undergo further high-level packaging into metaphase chromosomes. The relatively loosely packed form of chromatin wherein DNA is wrapped around histone proteins, but the histone proteins are not wrapped into 30 nm fibers, is known as euchromatin and is the form of chromatin that is typically associated with gene transcription. Conversely, the more densely packed form of chromatin, in which histones have wrapped into 30 nm fibers, is known as heterochromatin. The density of chromatin packaging in heterochromatin typically precludes the ability of RNA polymerases to access DNA and carry out transcription. Accordingly, epigenetic modifications of structural proteins in chromatin (e.g., histones), such as those produced by fusion proteins and other compositions of the present invention, control local chromatin structure (e.g., whether the chromatin is in the form of heterochromatin or euchromatin), which in turn affects a target chromatin site (e.g., gene) expression or activity.

“Histones,” which can be modified by fusion proteins and other compositions of the present invention, are highly alkaline proteins that are found in eukaryotic cells and, together with DNA, form the fundamental unit of chromatin known as the nucleosome. Histones function to increase chromatin packaging density, in part, by serving as a structure which DNA can wrap around. The five major families of histone proteins include H2A, H2B, H3, H4, and H1/H5. The latter family constitutes what are known as linker histones, while the first four families are known as core histones. The nucleosome core consists of two H2A-H2B dimers and an H3-H4 tetramer.

In mammals, there are several subfamilies of histone H3: H3.1, H3.2, H3.3, H3.4, H3.5, H3.X, and H3.Y. In humans, H3.1 histone proteins include those encoded by the HIST1H3A, HIST1H3B, HIST1H3C HIST1H3D, HIST1H3E, HIST1H3F, HIST1H3F, HIST1H3G, HIST1H3H, HIST1H3I, and HIST1H3J genes. H3.2 histone proteins in humans include those encoded by the HIST2H3A, HIST2H3C, and HIST2H3D genes. In humans, H3.3 histone proteins include those encoded by the H3F3A and H3F3B genes.

Various modifications of amino acid residues within a histone protein, such as those produced by fusion proteins and other compositions of the present invention, can affect the chemical properties of the histone, and by extension, affect processes such as chromatin packing. Lysine and arginine are residues modified within histone proteins. For example, lysine residues can be methylated or acetylated, or arginine residues can be methylated or citrullinated by fusion proteins of the present invention. Also, serine, threonine, and tyrosine residues can be phosphorylated by fusion proteins of the present invention. Acetylation, which is typically associated with increased transcriptional activity, can, for example, neutralize the positive charge or the lysine residue side chain, thus decreasing the electrostatic interaction between the histone protein and associated DNA molecules. While histone methylation can be associated with different chromatin packing states or levels of transcription activity, methylation of lysines 9 and 27 of histone H3 and lysine 20 of histone H4 are typically associated with suppressed transcription. In particular, dimethylation and trimethylation of lysine 9 of histone H3 (H3K9me2/3), trimethylation of lysine 27 of histone H3 (H3K27me3), and trimethylation of lysine 20 of histone H4 (H4K20me3) are associated with suppressed transcription.

In some embodiments, an epigenetic modification of a nucleic acid (e.g., DNA) component of chromatin at a target site is produced. In other embodiments, an epigenetic modification of a protein (e.g., a histone protein such as a histone H3 protein) component of chromatin at a target site is produced. In some embodiments, an epigenetic modification of a nucleic acid component and a protein component of chromatin at a target site are produced. When an epigenetic modification of a histone H3 protein is produced, in particular embodiments lysine 9 and/or lysine 27 of histone H3 are modified. In some embodiments, a fusion protein of the present invention removes an acetyl group from lysine 27 on histone H3. In some embodiments, a fusion protein of the present invention adds 1, 2, or 3 methyl groups to lysine 9 on histone H3. In other embodiments, a fusion protein of the present invention adds 1, 2, or 3 methyl groups to lysine 27 on histone H3. In particular embodiments, a fusion protein of the present invention adds 1, 2, or 3 methyl groups to lysine 9 on histone H3 and adds 1, 2, or 3 methyl groups to lysine 27 on histone H3. In some instances, lysine 9 on histone H3 is trimethylated (H3K9me3) and/or lysine 27 on histone H3 is trimethylated (H3K27me3). In some embodiments, a fusion protein deacetylates lysine 27 on histone H3 and methylates (e.g., trimethylates) lysine 27 on histone H3. In particular embodiments, the deacetylation event precedes the methylation event.

In some embodiments, epigenetic modification of a target chromatin site by a fusion protein of the present invention produces or is associated with a change in chromatin packing. In some instances, the epigenetic modification results in or is associated with heterochromatin formation (e.g., a transition from euchromatin to heterochromatin). In other instances, the epigenetic modification results in or is associated with euchromatin formation (e.g., a transition from heterochromatin to euchromatin). Such changes in chromatin packing can produce or be associated with a change in target chromatin site expression.

Chromatin immunoprecipitation (ChIP) assays and assays of epigenetic modification can be used to identify or confirm epigenetic modifications produced by fusion proteins of the present invention. ChIP assays are techniques that allow the detection of interactions between proteins and nucleic acids (e.g., DNA). ChIP assays can be used, for example, to detect interactions between DNA and transcription factors or chromatin-modifying proteins. ChIP assays can also be used to analyze the chromatin structure and epigenetic modifications at specific sites of interest (e.g., particular DNA sequences of interest). In one type of ChIP assay, commonly referred to as xChIP, formaldehyde is used to crosslink chromatin (i.e., DNA and associated proteins). Following crosslinking, DNA-protein complexes are immunoprecipitated (e.g., using antibodies specific for the protein(s) of interest). The crosslinks are then reversed, and the isolated DNA can be analyzed (e.g., by sequencing, PCR, or the detection of epigenetic modification such as a methylation assay. Another type of ChIP assay, commonly referred to as nChIP, uses nuclease digestion to prepare chromatin for analysis. nChIP assays allow for more accurate detection of epigenetic modification of histones such as methylation and acetylation than is typically possible with formaldehyde crosslinking, although nChIP assays do not always allow for the detection of DNA-protein interactions when the proteins have a weak binding affinity for DNA. Many ChIP assays are semi-quantitative, although in some cases it is desirable to couple a ChIP assay with a method such as quantitative PCR.

ChIP assays can also be combined with an assay to detect epigenetic modifications, such as DNA methylation assays. A non-limiting example of a DNA methylation assay is DNA bisulfite modification, wherein DNA obtained from a ChIP assay is treated with bisulfite and methylation-specific primers are used to detect changes in DNA methylation.

Changes in chromatin structure (e.g., arising from epigenetic modifications effected by fusion proteins of the present invention for producing epigenetic modifications) can be assessed by additional methods, non-limiting examples of which include DNasel hypersensitivity assays and trichostatin A (TSA) assays. DNasel hypersensitivity sites are typically located in or around promoter regions; as such DNasel hypersensitivity assays can be used to differentiate transcriptionally active from transcriptionally inactive chromatin regions. TSA, at low doses, inhibits the activity of histone deacetylases (HDACs). Accordingly, TSA assays can be used to determine the role that acetylation (or deacetylation) plays at a particular target chromatin site of interest (e.g., a gene of interest).

In some embodiments, epigenetic modification of a target chromatin site (e.g., a gene) by a fusion protein of the present invention produces or is associated with a reduction in, or suppression of, expression of the target chromatin site (e.g., gene expression is reduced or suppressed). In some instances, expression is reduced by at least about 1.1-, 1.2-, 1.3-, 1.4-, 1.5-, 1.6-, 1.7-, 1.8-, 1.9-, 2-, 2.1-, 2.2-, 2.3-, 2.4-, 2.5-, 2.6-, 2.7-, 2.8-, 2.9-, 3-, 3.1-, 3.2-, 3.3-, 3.4-, 3.5-, 3.6-, 3.7-, 3.8-, 3.9-, 4-, 4.1-, 4.2-, 4.3-, 4.4-, 4.5-, 4.6-, 4.7-, 4.8-, 4.9-, 5-, 5.1-, 5.2-, 5.3-, 5.4-, 5.5-, 5.6-, 5.7-, 5.8-, 5.9-, 6-, 6.1-, 6.2-, 6.3-, 6.4-, 6.5-, 6.6-, 6.7-, 6.8-, 6.9-, 7-, 7.1-, 7.2-, 7.3-, 7.4-, 7.5-, 7.6-, 7.7-, 7.8-, 7.9-, 8-, 8.1-, 8.2-, 8.3-, 8.4-, 8.5-, 8.6-, 8.7-, 8.8-, 8.9-, 9-, 9.1-, 9.2-, 9.3-, 9.4-, 9.5-, 9.6-, 9.7-, 9.8-, 9.9-, 10-, 10.5-, 11-, 11.5-, 12-, 12.5-, 13-, 13.5-, 14-, 14.5-, 15-, 15.5-, 16-, 16.5-, 17-, 17.5-, 18-, 18.5-, 19-, 19.5-, or 20-fold. The reduction in expression can be determined, for example, with respect to a control (e.g., expression of a target chromatin site that has not been epigenetically modified by the fusion protein of the present invention for which the comparison is being made).

In some embodiments, epigenetic modification of a target chromatin site (e.g., a gene) by a fusion protein of the present invention produces or is associated with an increase in, or exacerbation of, expression of the target chromatin site (e.g., gene expression is increased or exacerbated). In some instances, expression is increased by at least about 1.1-, 1.2-, 1.3-, 1.4-, 1.5-, 1.6-, 1.7-, 1.8-, 1.9-, 2-, 2.1-, 2.2-, 2.3-, 2.4-, 2.5-, 2.6-, 2.7-, 2.8-, 2.9-, 3-, 3.1-, 3.2-, 3.3-, 3.4-, 3.5-, 3.6-, 3.7-, 3.8-, 3.9-, 4-, 4.1-, 4.2-, 4.3-, 4.4-, 4.5-, 4.6-, 4.7-, 4.8-, 4.9-, 5-, 5.1-, 5.2-, 5.3-, 5.4-, 5.5-, 5.6-, 5.7-, 5.8-, 5.9-, 6-, 6.1-, 6.2-, 6.3-, 6.4-, 6.5-, 6.6-, 6.7-, 6.8-, 6.9-, 7-, 7.1-, 7.2-, 7.3-, 7.4-, 7.5-, 7.6-, 7.7-, 7.8-, 7.9-, 8-, 8.1-, 8.2-, 8.3-, 8.4-, 8.5-, 8.6-, 8.7-, 8.8-, 8.9-, 9-, 9.1-, 9.2-, 9.3-, 9.4-, 9.5-, 9.6-, 9.7-, 9.8-, 9.9-, 10-, 10.5-, 11-, 11.5-, 12-, 12.5-, 13-, 13.5-, 14-, 14.5-, 15-, 15.5-, 16-, 16.5-, 17-, 17.5-, 18-, 18.5-, 19-, 19.5-, or 20-fold. The increase in expression can be determined, for example, with respect to a control (e.g., expression of a target chromatin site that has not been epigenetically modified by the fusion protein of the present invention for which the comparison is being made).

Typically, epigenetic modifications produced by fusion proteins of the present invention will produce a decrease or increase in the level of mRNA expression (i.e., a decrease or increase in transcription of a gene expressed by the target chromatin site or under the control of a genetic regulatory element at the target chromatin site). Accordingly, the amount of a decrease or increase in expression can be determined or quantified by measuring mRNA levels (e.g., of a gene expressed by the target chromatin site or under the control of a genetic regulatory element at the target chromatin site). In some embodiments, the amount of a decrease or increase in expression is expressed as a fold change in the level of one or more mRNA transcripts. Exemplary methods for measuring mRNA levels include, without limitation, PCR (e.g., reverse-transcription quantitative PCR) and microarray analysis.

In addition, epigenetic modifications produced by fusion proteins of the present invention can produce changes in the level of protein expression. Accordingly, the amount of a decrease or increase in expression effected by an epigenetic modification can be determined or quantified by measuring protein levels (e.g., of a protein expressed from a gene expressed by the target chromatin site or under the control of a genetic regulatory element at the target chromatin site. In some embodiments, the amount of a decrease or increase in expression is expressed as a fold change in the level of one or more proteins. Exemplary methods for determining protein expression or quantifying the presence of other compounds (e.g., metabolites or other biochemicals that can be used to assay metabolic activity) include, without limitation, Western Blot, dot blot, enzyme-linked immunosorbent assay (ELISA), radioimmunoassay (RIA), immunoprecipitation, immunofluorescence, immunohistochemistry FACS analysis, chemiluminescence, and multiplex bead assays (e.g., using Luminex or fluorescent microbeads).

Epigenetic modifications produced according to compositions and methods of the present invention can produce changes in one or more phenotypes (e.g., the level or activity of a biochemical pathway, or the morphology or developmental fate of a cell or tissue). In some embodiments, the effects of epigenetic modifications can be assessed by employing a reporter or selectable marker to examine the phenotype of an organism or a population of organisms. In some instances, the marker produces a visible phenotype, such as the color of an organism or population of organisms. As a non-limiting example, the phenotype can be examined by growing the target organisms (e.g., cells or other organisms that have had their genome epigenetically modified) and/or their progeny under conditions that result in a phenotype, wherein the phenotype may not be visible under ordinary growth conditions.

In some embodiments, the reporter or selectable marker, used for assessing the effects of an epigenetic modification made by a fusion protein of the present invention, is a fluorescent tagged protein, an antibody, a labeled antibody, a chemical stain, a chemical indicator, or a combination thereof. In other embodiments, the reporter or selectable marker responds to a stimulus, a biochemical, or a change in environmental conditions. In some instances, the reporter or selectable marker responds to the concentration of a metabolic product, a protein product, a synthesized drug of interest, a cellular phenotype of interest, a cellular product of interest, or a combination thereof. A cellular product of interest can be, as a non-limiting example, an RNA molecule (e.g., messenger RNA (mRNA), long non-coding RNA (lncRNA), microRNA (miRNA)), which can be produced, for example, under the control of a target chromatin site that is epigenetically modified by a fusion protein of the present invention.

In some embodiments, an epigenetic modification is produced in vitro. In other embodiments, the fusion protein and the target chromatin site are in a cell. As a non-limiting example, the fusion protein, or a combination of the fusion protein and a gRNA, can be introduced into a cell, and the fusion protein subsequently produces an epigenetic modification at a target chromatin site (e.g., a target chromatin site that is present within the cell's genome). Alternatively, a nucleic acid or a vector comprising a polynucleotide sequence encoding the fusion protein and/or the gRNA can be introduced into a cell, and subsequently the fusion protein can be expressed by the cell. The expressed fusion protein can then produce an epigenetic modification at a target chromatin site within the cell.

Epigenetic modification methods of the present invention can be performed in a multiplex format. In some embodiments, multiplexing comprises introducing two or more gRNA molecules into a host cell, or cloning two or more nucleic acids comprising polynucleotide sequences that encode gRNA molecules in tandem into a single expression vector (i.e., an expression vector that is subsequently introduced into a host cell). In some instances, at least about 2, 3, 4, 5, 6, 7, 8, 9, 10, or more gRNA molecules are introduced into a host cell. In some embodiments, at least about 2, 3, 4, 5, 6, 7, 8, 9, 10, or more polynucleotide sequences that encode gRNA molecules (e.g., different gRNA molecules) are included in a single vector. In some embodiments, at least 2, 3, 4, 5, 6, 7, 8, 9, 10, or more expression vectors are introduced into a host cell. Each of the expression vectors can encode one or more different gRNA molecules.

In still other embodiments, multiplexing comprises transfecting a plurality of host cells. Each host cell can be transfected with a single expression vector or multiple different expression vectors. In some embodiments, a plurality of host cells comprises about 10³, about 10⁴, about 10⁵, about 10⁶, about 10⁷, or about 10⁸ cells. Also, multiple embodiments of multiplexing can be combined.

By using one or a combination of the various multiplexing embodiments, it is possible to epigenetically modify any number of target sites within a genome. In some instances, at least about 10 (e.g., at least about 1, 2, 3, 4, 5, 6, 7, 8, 9, or 10) target sites are modified. In other instances, between about 10 and 100 (e.g., about 10, 15, 20, 25, 30, 35, 40, 45, 50, 55, 60, 65, 70, 75, 80, 85, 90, 95, or 100) target sites are modified. In some instances, about 100 and about 1,000 (e.g., about 100, 200, 300, 400, 500, 600, 700, 800, 900, or 1,000) target sites are modified. In other instances, between about 1,000 and about 30,000 (e.g., about 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 21,000, 22,000, 23,000, 24,000, 25,000, 26,000, 27,000, 28,000, 29,000, or 30,000) target sites are modified.

In some embodiments, more than one gRNA (e.g., 2, 3, 4, 5, 6, 7, 8, 9, 10, or more) molecule is used modify each target site. In some instances, a multiplexed experiment utilizes at least about 2 to about 100 (e.g., at least about 2, 3, 4, 5, 6, 7, 8, 9, 10, 20, 30, 40, 50, 60, 70, 80, 90, or 100) different gRNA molecules. In other instances, a multiplexed experiment utilizes at least about 100 to about 10,000 (e.g., at least about 100, 200, 300, 400, 500, 600, 700, 800, 900, 1,000, 1,500, 2,000, 2,500, 3,000, 3,500, 4,000, 4,500, 5,000, 5,500, 6,000, 6,500, 7,000, 7,500, 8,000, 8,500, 9,000, 9,500, or 10,000) different gRNA molecules. In some instances, a multiplexed experiment utilizes at least about 10,000 to about 500,000 (e.g., at least about 10,000, 20,000, 30,000, 40,000, 50,000, 60,000, 70,000, 80,000, 90,000, 100,000, 150,000, 200,000, 250,000, 300,000, 350,000, 400,000, 450,000, or 500,000) different gRNA molecules.

In some embodiments, the host cell comprises a population of cells (e.g., host cells). In some instances, one or more epigenetic modifications are produced in at least about 20 percent (e.g., at least about 20, 21, 22, 23, 24, 25, 26, 27, 28, 29, 30, 35, 40, 45, 50, 55, 60, 65, 70, 75, 80, 85, 90, 95, or 100 percent) of the population of cells. In other instances, one or more epigenetic modifications are produced in at least about 50 percent (e.g., at least about 50, 51, 52, 53, 54, 55, 56, 57, 58, 59, 60, 65, 70, 75, 80, 85, 90, 95, or 100 percent) of the population of cells. In still other instances, one or more epigenetic modifications are produced in at least about 75 percent (e.g., at least about 75, 76, 77, 78, 79, 80, 81, 82, 83, 84, 85, 86, 87, 88, 89, 90, 95, or 100 percent) of the population of cells. In other instances, one or more epigenetic modifications are produced in at least about 90 percent (e.g., at least about 90, 91, 92, 93, 94, 95, 96, 97, 98, 99, or 100 percent) of the population of cells. In particular instances, one or more epigenetic modifications are produced in at least about 95 percent (e.g., at least about 95, 96, 97, 98, 99, or 100 percent) of the population of cells.

### VII. METHODS FOR TARGET SITE SCREENING AND OTHER APPLICATIONS

The compositions and methods of the present invention can be used to screen for one or more target chromatin sites (e.g., within the genome of a cell or organism). As a non-limiting example, compositions and methods of the present invention can be used to produce epigenetic modification(s) at one or more target chromatin sites, and then the effects of the epigenetic modification(s) on the expression of the target site(s) (e.g., one or more genes) can be assessed. Target site expression can be assessed in terms of transcriptional activity (e.g., mRNA levels), translational activity (e.g., protein levels), or phenotype, using techniques that are described herein and will be known to one of skill in the art.

Screening methods can be performed in a multiplex format as described herein. In some embodiments, multiplexed screening comprises introducing two or more gRNA molecules into a host cell, or cloning two or more nucleic acids comprising polynucleotide sequences that encode gRNA molecules in tandem into a single expression vector. In some instances, at least about 2 to about 10 gRNA molecules are introduced into a host cell. In some embodiments, at least about 2 to about 10 polynucleotide sequences that encode gRNA molecules (e.g., different gRNA molecules) are included in a single vector (i.e., a vector that is introduced into a host cell). In some embodiments, at least about 2 to about 10, or more expression vectors are introduced into a host cell. Each of the expression vectors can encode one or more different gRNA molecules.

In still other embodiments, multiplexed screening comprises transfecting a plurality of host cells. Each host cell can be transfected with a single expression vector or multiple different expression vectors. In some embodiments, a plurality of host cells comprises between about 10³ and about 10⁸ cells. Also, multiple embodiments of multiplexed screening can be combined. One of skill in the art will recognize that the progeny of epigenetically modified cells can also be used for screening according to methods of the present invention.

By using one or a combination of the various multiplexing embodiments, it is possible to screen any number of target sites within a genome. In some instances, at least about 10 to about 30,000 loci are screened. In some embodiments, more than one gRNA molecule is used to screen each locus. In some instances, a multiplexed screening experiment utilizes at least about 2 to about 500,000 different gRNA molecules.

The compositions and methods provided by the present invention are useful for any number of applications. As non-limiting examples, epigenetic modifications (e.g., of a genome) can be performed in order to prevent or treat a disease, or to identify one or more specific target chromatin sites (e.g., genetic loci) that contribute to a phenotype, disease, biological function, and the like. As another non-limiting example, epigenetic modifications for the purposes of screening according to the compositions and methods of the present invention can be used to improve or optimize a biological function or pathway.

The compositions and methods of the present invention are useful for preventing or treating any number of genetic diseases (e.g., in a subject in need thereof). The present invention is particularly well-suited for the prevention or treatment of diseases that result from the underexpression or overexpression of a gene product, such as a protein or enzyme. The present invention is also particularly well-suited for the prevention or treatment of diseases that arise from abnormal cell differentiation or development, as many of these processes are under the direct control of epigenetic regulation.

In some embodiments, the subject is treated (e.g., a target chromatin site in the subject is epigenetically modified) before any symptoms or sequelae of the genetic disease develop. In other embodiments, the subject has symptoms or sequelae of the genetic disease. In some instances, treatment results in a reduction or elimination of the symptoms or sequelae of the genetic disease.

In some embodiments, treatment (e.g., epigenetic modification of a target chromatin site) includes administering compositions (e.g., fusion proteins, nucleic acids, expression vectors, or cells) of the present invention directly to a subject. As a non-limiting example, pharmaceutical compositions of the present invention (e.g., comprising a fusion protein, nucleic acid, expression vector, or cell of the present invention and a pharmaceutically acceptable carrier) can be delivered directly to a subject (e.g., by local injection or systemic administration). In other embodiments, the compositions of the present invention are delivered to a host cell or population of host cells, and then the host cell or population of host cells is administered or transplanted into the subject. The host cell or population of host cells can be administered or transplanted with a pharmaceutically acceptable carrier. In some instances, epigenetic modification of the target chromatin site (e.g., of the host cell genome) has not yet been completed prior to administration or transplantation to the subject. In other instances, epigenetic modification of the target chromatin site has been completed when administration or transplantation occurs. In certain instances, progeny of the host cell or population of host cells are transplanted into the subject. In some embodiments, correct epigenetic modification of the host cell or population of host cells, or the progeny thereof, is verified before administering or transplanting cells containing modified chromatin or the progeny thereof into a subject. Procedures for transplantation, administration, and verification of correct epigenetic modification are discussed herein and will be known to one of skill in the art.

Compositions of the present invention, including cells and/or progeny thereof that have had their target chromatin sites epigenetically modified by the methods and/or compositions of the present invention, may be administered as a single dose or as multiple doses, for example two doses administered at an interval of about one month, about two months, about three months, about six months or about 12 months. Other suitable dosage schedules can be determined by a medical practitioner.

### VIII. KITS

In another aspect, the present invention provides kits for producing epigenetic modifications at a target chromatin site comprising a Cas nuclease (e.g., Cas9) recognition site, the kit comprising one or more fusion proteins of the present invention. The kit may also comprise one or more nucleic acids (e.g., encoding a fusion protein of the present invention), one or more expression vectors (e.g., comprising a nucleic acid comprising a polynucleotide sequence encoding a fusion protein of the present invention), or one or more cells (e.g., transfected with a nucleic acid or expression vector) of the present invention. The kit may further comprise guide RNA (gRNA) molecule(s), or nucleic acids or expression vectors containing polynucleotide sequences encoding the gRNA molecule(s).

Kits of the present invention can be packaged in a way that allows for safe or convenient storage or use (e.g., in a box or other container having a lid), Typically, kits of the present include one or more containers, each container storing a particular kit component such as a reagent, a control sample, and so on. The choice of container will depend on the particular form of its contents, e.g., a kit component that is in liquid form, powder form, etc. Furthermore, containers can be made of materials that are designed to maximize the shelf-life of the kit components. As a non-limiting example, kit components that are light-sensitive can be stored in containers that are opaque.

In some embodiments, the kit contains one or more reagents. In some instances, the reagents are useful for transfecting a host cell with a nucleic acid (e.g., encoding a fusion protein of the present invention), expression vector (e.g., comprising a nucleic acid of the present invention), or a plurality thereof, and/or inducing expression from the nucleic acid(s) and/or expression vector(s). The kit may further comprise one or more reagents useful for delivering fusion proteins of the present invention into a host cell. In yet other embodiments, the kit further comprises instructions for use.

### IX. EXAMPLES

The present invention will be described in greater detail by way of a specific example. The following example is offered for illustrative purposes only, and is not intended to limit the invention in any manner.

### Example 1

### dCas9-Based Epigenome Editing

This example demonstrates the use of fusion proteins of the present invention for producing epigenetic modifications of target chromatin sites. In particular, a broad set of epigenetic enzymes (epigenetic writers) and epigenetic recruiters (peptides or proteins recruiting chromatin modifying complexes) were investigated for their ability to produce transcriptionally repressive histone marks when fused to a catalytically inactive Cas9 (dCas9) platform. In addition to the writers of H3K9me3 (i.e., G9A, SUV39H1) and the KRAB repressor domain (6, 30), fusions to Ezh2 (i.e., a writer of H3K27me3) and to the N-terminal 45 residues of FOG1 (which has been associated with acquisition of H3K27me3 and loss of histone acetylation (31, 32)) were also created and used; these domains had not been previously investigated as dCas9 fusions. The effects of the marks introduced by these proteins on gene expression were compared to the effects of DNA methylation by dCas9-DNMT3A. This example shows that dCas9 fusions to catalytic domains of EZH2, G9A and SUV39H1, as well as dCas9 fused to the N terminus of FOG1, were sufficient for some level of repression of three different promoters in two different cell types, but that repression was not always correlated with the expected histone modification. This example also shows that the dCas9-like targeting protein dCpf1 was not able to substitute for dCas9 in these experiments. Finally, this example shows that combinations of targeted effectors were able to produce persistent silencing.

### Materials and Methods

Construction of dCas9 Expression Plasmids

A variety of epigenetic effectors were fused to human codon-optimized and catalytically inactive “dead” Cas9 (dCas9) in different conformations. The improved pCDNA3-dCas9 expression plasmid was obtained by altering the original dCas9 plasmid (33) using Gibson cloning. The improved pCDNA3-dCas9 contained two nuclear localization signals (NLS), a 3× FLAG epitope tag, and [(GGS)₅] (SEQ ID NO:75) amino acid linkers at the N- and C-termini of dCas9 with flanking restriction sites KpnI and NheI, respectively. The improved dCas9 protein sequence is set forth in SEQ ID NO:8. Effector domains were amplified using 2× Phusion Master Mix (New England Biolabs) according to the manufacturer's instructions. PCR primers for cDNA amplification of individual effector domains were designed with cloning vector overhangs for Gibson cloning. Primer sequences are set forth in SEQ ID NOS:11-22 and 27-34. cDNA for G9A[SET], SUV[SET], and DNMT3A was kindly provided by the lab of Marianne Rots (29, 34). The DNMT3L expression plasmid pCDNA-DNMT3L was a kind gift from Dr. Fred Chedin (35). Mouse Ezh2[FL] cDNA was synthesized by Bio Basic, Inc. Ezh2[FL] was used as a template to amplify the shorter Ezh2[SET] domain. Catalytic mutants Ezh2[SET-Y641A]-dCas9 and Ezh2[SET-Y726F]-dCas9 were created by site-directed mutagenesis using the QuikChange II XL Site-Directed Mutagenesis kit (Stratagene). The sequences of primers used for mutagenesis are set forth in SEQ ID NOS:23-26. The KRAB domain was amplified from dCas9-KRAB (33) and FOG1 cDNA was amplified from HEK293FT cells. Total RNA was isolated from HEK293FT using the RNeasy mini kit (Qiagen) and cDNA was synthesized using random hexamer primers using the RevertAid cDNA synthesis kit (ThermoScientific). Using Gibson Assembly (New England Biolabs), amplified cDNAs were cloned into either KpnI or NheI digested dCas9 for N-terminal or C-terminal fusions to dCas9, respectively. Finally, the FOG1 epigenetic effector construct was Gibson assembled (New England Biolabs). Protein sequences of dCas9-fusions are set forth in SEQ ID NOS:9 and 10. For arrays of two, three, and four FOG1 domains to the N-terminus of dCas9, FOG1 monomer coding sequences were amplified separately by PCR introducing a GS linker between individual monomer coding sequences and the KpnI and FseI restriction sites at the beginning of first monomer and the end of the last monomer for each array. In addition, a BsaI endonuclease site was added to either end of the FOG1 monomers and each fragment contained a distinct 4-base overhang that directed the assembly of multiple monomers. The sequences of amplification primers are set forth in SEQ ID NOS:35-42. Two, three, or four monomer coding sequences were mixed with pFusA plasmid for Golden Gate Assembly cloning with BsaI and T4 DNA ligase (New England Biolabs). DNA fragments of arrays of two, three, and four FOG1 domains were digested with KpnI and FseI and ligated into the KpnI/FseI digested dCas9 plasmid.

### Cloning of Expression Plasmid

The cloning vector was obtained from Addgene (36; Addgene, plasmid #41824) and was linearized using the AflII restriction enzyme. 19-bp gRNA target sequences were selected within 500 base pairs of the relevant gene promoter using the online tool CHOPCHOP (37). Each gRNA sequence was selected and incorporated into two 60-mer oligonucleotides that contained cloning vector overhangs for Gibson assembly. After annealing and extending the oligonucleotides to 100-bp, the PCR purified (PCR purification kit; QIAGEN) dsDNA was Gibson assembled into the AflII linearized plasmid. The sequences of oligomers used to create target specific vectors are set forth in SEQ ID NOS:43-45.

Construction of dCpf1 Expression Plasmids and crRNA

The inactive Cpf1 was generated by mutating the catalytic domain AsCpf1 (D908A; (38)). This amino acid change was induced through adding mutations in the primers during PCR amplification with pcDNA3.1-hAsCpf1 (Addgene, plasmid #69982) as template. Primer sequences are set forth in SEQ ID NOS:49-52. Two PCR fragments were inserted into the FseI/NheI linearized pCDNA3-dCas9 backbone using Gibson assembly, thereby replacing dCas9 with dCpf1. Effector domains were then added using KpnI and/or NheI digested plasmid to generate N- and/or C-terminal dCpf1 fusions following the same principle as dCas9 fusions. This step used the same cDNA amplification primers as described for dCas9 fusions. crRNA was designed to target 23-bp adjacent to the 5′-NTTT-3′ PAM. crRNA target sequences are listed in SEQ ID NO:46-48. For Cpf1 cleavage assays and dCpf1 ChIP assays, the U6-crRNA cassette was amplified by PCR (39). The U6-crRNA cassette was then co-transfected with dCpf1 expressing plasmids as described below. To determine repression by dCpf1 fusion proteins plasmids containing the U6-crRNA cassette were coexpressed with plasmids expressing dCpf1 fusions (40).

### Cell Lines and Transfection

The human colon cancer cell line HCT116 (ATCC #CCL-247) was grown in McCoy's 5A Medium supplemented with 10% fetal bovine serum (FBS) and 1% penicillin/streptomycin. Cells were maintained at 37° C. and 5% CO₂. HCT116 cells were authenticated by the Bioreagent and Cell Culture Core, USC Norris Comprehensive Cancer Center. Cells of 50-60% confluency were transfected using Lipofectamine 3000 (Life Technologies) following the manufacturer's instructions. Transfections for RNA extraction were performed in 12-well plates using 625 ng dCas9 expression vector, 500 ng of equimolar pooled expression vectors, and 125 ng pBABE-puro. Transfections with dCpf1 were carried out using the same protocol except that U6-crRNA expressing plasmids were co-transfected with dCpf1 expressing plasmids as described elsewhere (39). For ChIP assays and DNA-methylation analysis, cells were plated in 10-cm² culture dishes and transfection was scaled up accordingly. Transfection medium was replaced 24 hours post-transfection with growth medium containing 3 μg/mL puromycin to enrich for transfected cells. Subsequently, puromycin-containing media was exchanged every 24 hours. To assay for persistent repression, media was switched to standard growth media four days after transfection.

### RNA Extraction and Reverse-Transcription Quantitative PCR (RT-qPCR)

Transfected cells were rinsed in 1× DPBS and RNA stabilized by adding 500 μg RNAlater (Ambion) and stored at 4° C. for up to one week. Total RNA was extracted 3-4 days after transfection using the RNeasy Mini kit (QIAGEN) and 500 ng RNA were reverse-transcribed using the SuperScript VILO MasterMix (Invitrogen) according to the manufacturer's instructions. Real-time PCR was performed in triplicate with 2× iQ SYBR mix (BioRad) using the CFX384 Real-Time System C1000 Touch Thermo Cycler (BioRad) and the included software was used to extract raw Cq values. Gene expression analysis was performed with GAPDH as a reference gene in at least two biological replicates using intron-spanning HER2 primers (HER2-F 5′-GGGAAACCTGGAACTCACCT-3′ (SEQ ID NO:53); HER2-R 5′-GACCTGCCTCACTTGGTTGT-3′ (SEQ ID NO:54)), EPCAM primers (EPCAM-F 5′-CTGGCCGTAAACTGCTTTGT-3′ (SEQ ID NO:55); EPCAM-R 5′-TCCCAAGTTTTGAGCCATTC-3′ (SEQ ID NO:56)), MYC primers (MYC-F 5′-AAACACAAACTTGAACAGCTAC-3′ (SEQ ID NO:57); MYC-R 5′-ATTTGAGGCAGTTTACATTATGG-3′ (SEQ ID NO:58)) and GAPDH primers (GAPDH-F 5′-AATCCCATCACCATCTTCCA-3′ (SEQ ID NO:59); GAPDH-R 5′-CTCCATGGTGGTGAAGACG-3′ (SEQ ID NO:60)). Relative target gene expression was calculated as the difference between the target gene and the GAPDH reference gene (i.e., dCq=Cq[target]−Cq[GAPDH]). Gene expression results are indicated as fold change relative to a reference sample (usually dCas9 without any effector domain), using the ddCq method. A one-way ANOVA (ANalysis Of VAriance) with post-hoc Tukey HSD (Honestly Significant Difference) test was applied to determine statistical significance for different dCas9 fusions.

### Chromatin Immunoprecipitation (ChIP)-qPCR

For ChIP assays of histone marks, transfected cells were cross-linked 3-4 days post transfection by incubation with 1% formaldehyde solution for 10 minutes at room temperature and the reaction was stopped by the addition of glycine to a final concentration of 125 mM. Cross-linked cell pellets were stored at −80° C. Chromatin was extracted and ChIP was performed using StaphA cells (Sigma-Aldrich, St. Louis, MO, USA) to collect the immunoprecipitates as previously described (33,41). Briefly, chromatin was sheared to an average fragment size of 500-bp using a Bioruptor 2000 (Diagenode). 10 μg chromatin were used per ChIP assay. ChIP enrichment was performed by incubation with 3μg H3K9me3 antibody (Abcam ab8898), 3 H3K9me2 antibody (MP 07-441), 2μg H3K27me3 antibody (MP 07-449), 2μg H3K27ac antibody (Active Motif #39133), or 2μg normal rabbit IgG (Abcam ab46540) for 16 hours at 4° C. Immuno complexes were bound to StaphA cells for 15 minutes at room temperature. For dCpf1 and dCas9 ChIP assays, HCT116 cells were transfected in 10 cm culture dishes as described above, but puromycin selection was omitted. After cross-linking of chromatin, ChIP assays were performed using 3 μg FLAG antibody (SIGMA M2 F1804) at 4° C. overnight. Immuno complexes were captured with 3μg rabbit anti-mouse antibody for 1 hour at 4° C. and were bound to StaphA cells for 15 minutes at room temperature. After washing and reversal of cross-links, DNA was purified using the QIAquick PCR Purification Kit (Qiagen). ChIP-DNA and diluted input control were used for subsequent qPCR reactions with 2x SYBR FAST Master Mix (KAPA Biosystems) according to the manufacturer's recommendations using the CFX384 Real-Time System C1000 Touch Thermo Cycler (BioRad). ChIP enrichment was calculated relative to input samples using the dCq method (i.e., dCq=Cq[HER2-ChIP]-Cq[input]). HER2 ChIP amplification primers are as follows: HER2-ChIP-F (5′-TTGGAATGCAGTTGGAGGGG-3′ (SEQ ID NO:61)) and HER2-ChIP-R (5′-GGTTTCTCCGGTCCCAATGG-3′ (SEQ ID NO:62)). A one-way ANOVA (ANalysis Of VAriance) with post-hoc Tukey HSD (Honestly Significant Difference) test was applied to determine statistical significance for different dCas9 fusions.

### DNA Methylation Analysis

Genomic DNA from transfected and untreated cells was isolated using the Quick-gDNA MiniPrep kit (ZYMO). Bisulfite conversion was performed using the EZ DNA Methylation-Lightning Kit (ZYMO) following the manufacturer's instructions. Bisulfite-Sequencing PCR primers (HER2-BSP-F 5′-GGAGGGGGTAGAGTTATTAGTTTTT-3′ (SEQ ID NO:63) and HER2-BSP-R 5′-AAATAACAACTCCCAACTTCACTTT-3′ (SEQ ID NO:64)) were designed using MethPrimer (42). Bisulfite converted DNA was used for PCR amplification with GoTaq polymerase (Promega) and the 152-bp PCR product was purified with the QIAquick PCR Purification Kit (Qiagen). Amplicons were inserted into the pCR4-TOPO TA vector using the TOPO-TA-cloning kit (ThermoFisher) and transformed into NEB5α competent cells. Plasmid DNA from individual recombinant clones was isolated and subjected to Sanger sequencing using M13F primers at the College of Biological Sciences UC DNA Sequencing Facility. Methylation status of CpGs for each clone was determined by sequence comparison.

### Single-Strand Annealing (SSA) Recombination Reporter Assay

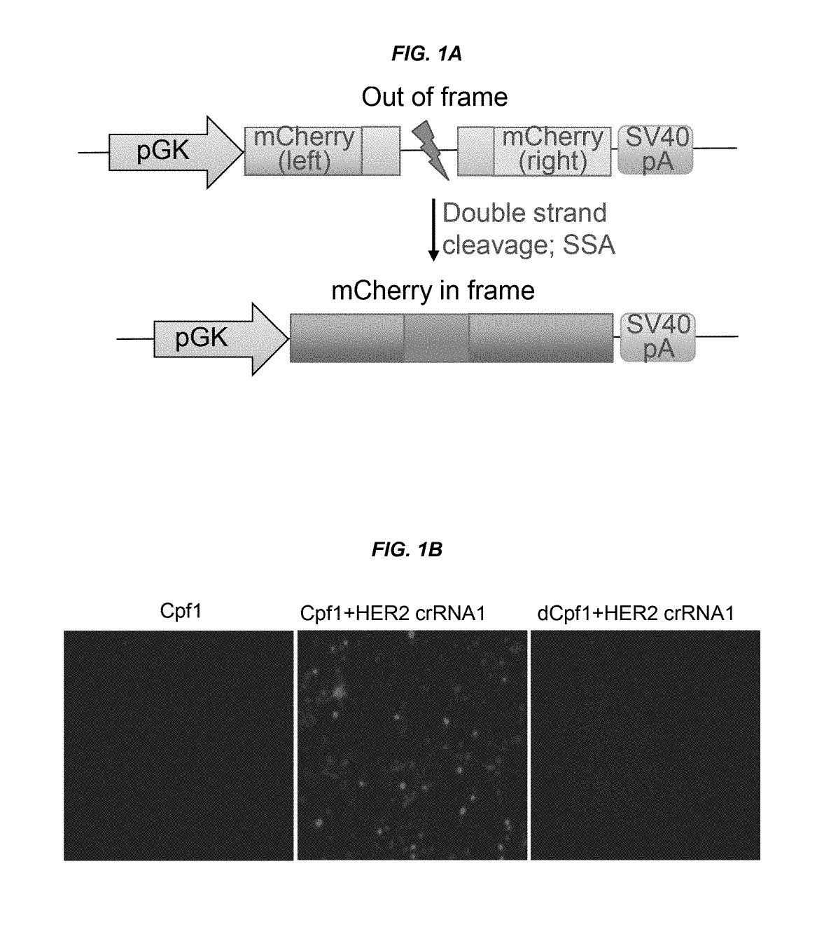

For the pPGK-mCherry reporter plasmid, the Cpf1 nuclease binding site (crRNA binding region on HER2 promoter) was inserted between XhoI/BamHI sites, which are flanked by 200-bp direct repeats derived from mCherry as single-strand annealing (SSA) arms (43). The ORF of the mCherry gene was interrupted by the insertion of the relevant binding region and a series of three stop codons (FIG. 1A). Nucleases causing double strand breaks at the target site induced SSA repair, which led to expression of functional mCherry protein that could readily be detected by its fluorescence (FIG. 1B). To evaluate cleavage activity, pcDNA3-Cpf1 and pcDNA3-dCpf1, were co-transfected with the three PCR amplified U6-crRNAs cassette and the mCherry reporter plasmid in HEK293T cells. Cells were observed 48 hours post transfection.

### Western Blot Analysis

Transfected cells were lysed 48 hours post transfection in 1× RIPA buffer (Millipore) supplemented with protease inhibitor cocktail (Roche). Protein concentrations were determined by Bradford assay (BioRad) and 20 μg protein were separated on a 4-15% TGX gel (BioRad) in Tris/Glycine/SDS buffer and transferred onto nitrocellulose membranes. Protein loading was evaluated by Ponceau S stain. After rinsing the membrane with deionized water, non-specific antigen binding was blocked in TBST (50 mM Tris, 150 mM NaCl, and 0.1% Tween-20) with 5% nonfat dry milk (Cell Signaling). Membranes were incubated with primary antibody in blocking solution at 4° C. overnight. Monoclonal antibodies against FLAG (1:1000; SIGMA M2 F1804) or anti-beta-actin (1:2500; SIGMA A5441) were used. Membranes were washed with TBST three times for 10 minutes before membranes were incubated with HRP conjugated anti-mouse secondary antibody at room temperature. After 45 minutes, the membrane was washed three times in TBST and proteins were visualized with Amersham ECL Prime Western Blotting Detection Reagent (GE Healthcare) and autoradiobiography film.

### Results

Systematic Evaluation of Repression by dCas9 Fused to Catalytic Domains of Histone Lysine Methyltransferases G9A and SUV39H1

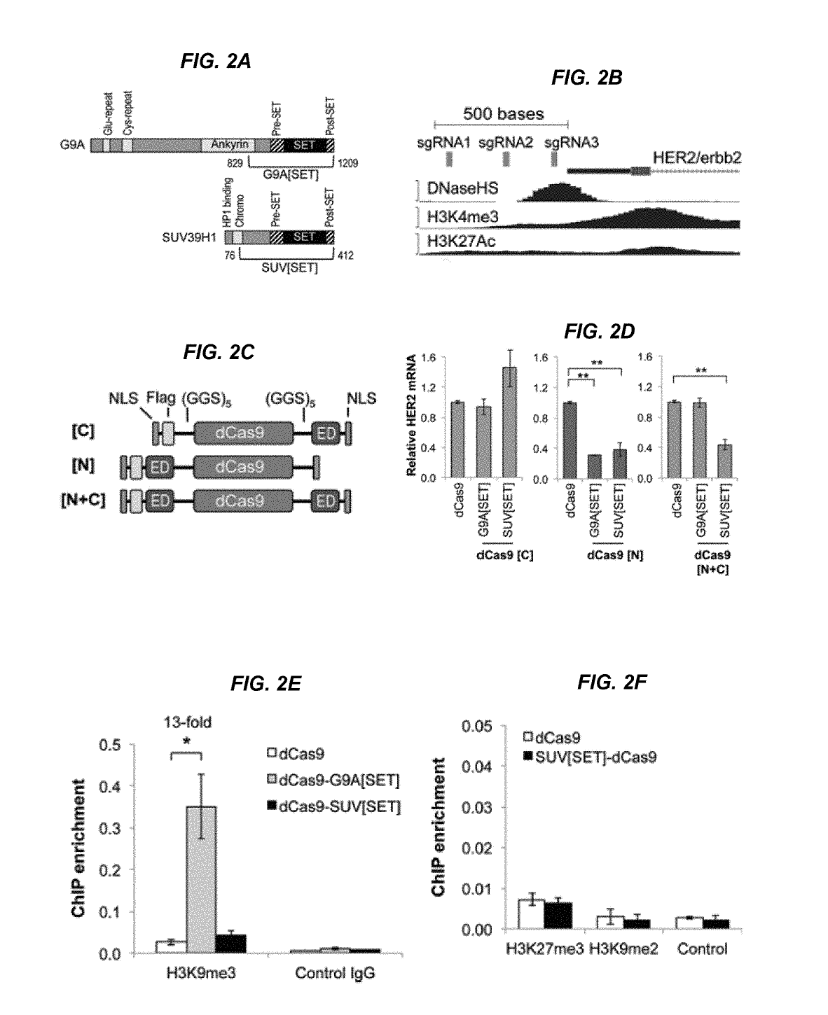

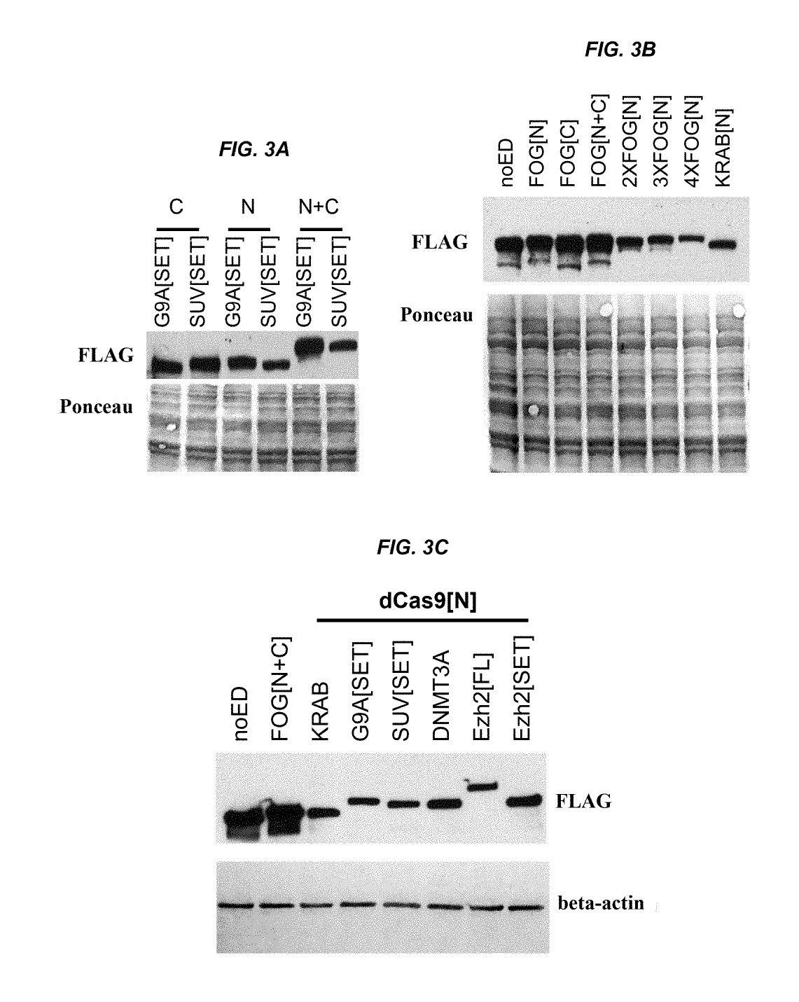

Epigenetic effector domains for H3K9 methylation have been previously fused to artificial zinc finger proteins (ZFP) to affect transcriptional regulation in a targeted manner. More specifically, the C-terminal end of ZFP E2C, which targets the HER2 promoter, had been previously fused to the catalytic SET domains of the histone methyltransferases G9A or SUV39H1 (herein referred to as G9A[SET] and SUV[SET], respectively; FIG. 2A), and was shown to repress endogenous HER2 gene expression (29). In order to test the framework for the repressive and epigenetic activity of RNA-guided dCas9 fusions, G9A[SET] and SUV[SET] were fused to dCas9 and used simultaneously to target the dCas9 fusions to the promoter of HER2 (FIG. 2B; target site sequences set forth in SEQ ID NOS:43-45). Effector domains were fused to either the N-terminus, the C-terminus or both the N- and C-termini of dCas9 to determine the most effective configuration for the dCas9-fusions (FIG. 2C). Crystal structures have revealed that neither the N-terminus nor C-terminus of dCas9 are in immediate proximity to its bound DNA (44). Therefore, a 15-amino acid linker (i.e., (GGS)₅) (SEQ ID NO:75) was introduced between dCas9 and the effector domain to improve the ability of the effector domain to contact the DNA or histones. Surprisingly, it was found that the domains fused to the C-terminal end of dCas9 were unable to repress transcription, whereas both N-terminal fusions of G9A[SET]-dCas9 and SUV[SET]-dCas9 displayed 3.3-fold and 2.7-fold downregulation of HER2 mRNA, respectively (Tukey HSD test, P<0.01; FIG. 2D). Western blot analysis confirmed that N- and C-terminal dCas9-fusions were expressed at similar levels (FIG. 3A) and that differences in repressive activity were due to the configuration of the dCas9 fusions. Having effector domains at both the N- and C-termini did not increase the repressive capacity. Specifically, the repressive capacity of SUV[SET]-dCas9-SUV[SET] (2.2-fold) was comparable to that of the single SUV[SET]-dCas9, while G9A[SET]-dCas9-G9A[SET] showed no repressive activity, suggesting that the C-terminal G9A[SET] attenuated the activity of the N-terminal fusion. Negative controls using a dCas9 with no effector domains but co-transfected with the three guide-RNAs, or an mCherry reporter plasmid only, had no effect on HER2 expression. Since N-terminal fusions of effector domains to dCas9 were most effective, these were the focus of subsequent experiments.

Repression by dCas9-SUV[SET] Does Not Require Trimethylation Of H3K9 at the HER2 Gene Promoter

To determine if repression by G9A[SET]-dCas9 and SUV[SET]-dCas9 was associated with the trimethylation of H3K9, histone ChIP-qPCR assays were performed to quantitatively measure H3K9me3 enrichment at the HER2 promoter. ChIP enrichment was evaluated relative to dCas9 that did not contain an effector domain. G9A[SET]-dCas9 co-transfected with the three guide-RNAs produced a 13-fold increase in H3K9 trimethylation compared to dCas9 with no ED (Tukey HSD test, P<0.05; FIG. 2E), whereas SUV[SET]-dCas9 did not increase H3K9me3 levels. This result was surprising given that G9A[SET]-dCas9 and SUV[SET]-dCas9 caused similar levels of HER2 repression (3.3-fold and 2.7-fold, respectively, FIG. 2D). Therefore, although the SUV[SET] domain was sufficient to repress HER2 transcription, it was not sufficient to mediate H3K9me3 addition. Importantly, the data suggest that an increase in H3K9me3 at the target promoter was not required for SUV-mediated repressive activity. Thus, some other activity of the SUV[SET] domain may have been responsible for the repression, since dCas9 alone did not cause repression. One possibility is that other repressive histone marks were deposited to cause the repression. This latter possibility was investigated by examining alternative histone marks that have been associated with repression. Neither H3K27me3 nor H3K9me2 marks changed at the HER2 promoter when targeted by SUV[SET]-dCas9 (for which H3K9me3 was expected but not observed) (FIG. 2F). The lack of deposition of expected or alternative repressive histone marks further supported the conclusion that repression by SUV[SET]-dCas9 did not require histone methylation.

Full-Length Histone Methyltransferase Ezh2 is Required for H3K27 Methylation, but H3K27me3 is Not Correlated with Repressive Activity

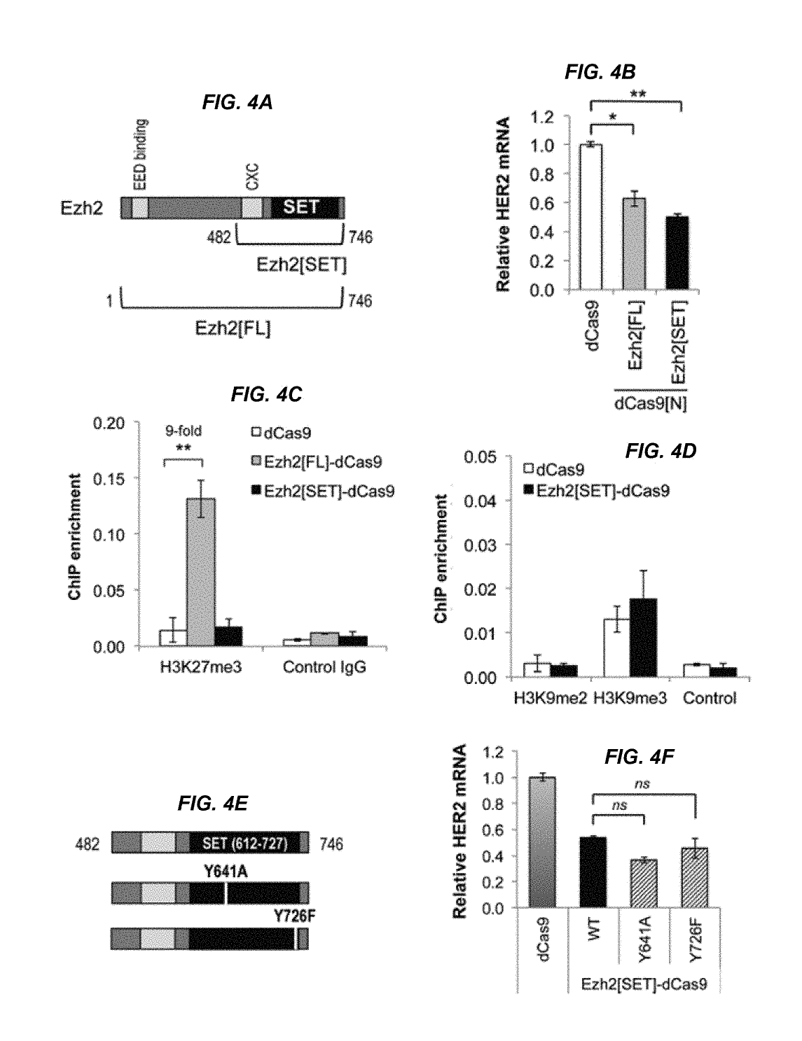

H3K9me3 and H3K27me3 mark distinct regions in the genome (45); H3K9me3 is a mark typical of constitutive heterochromatin, while H3K27me3 is usually enriched on facultative heterochromatin (2, 46). Since enzymes mediating the repressive H3K27me3 mark had not yet been targeted to a specific genomic locus by dCas9, dCas9 N-terminal fusions were created with the full-length mouse methyltransferase (Ezh2[FL]), as well as a truncated form (Ezh2[SET]) containing the CXC and SET domains (aa482-746) but lacking some of the N-terminal domains (FIG. 4A). Both Ezh2[FL]-dCas9 and Ezh2[SET]-dCas9 produced repression of HER2 gene expression (1.6-fold (Tukey HSD Test, P<0.05) and 2-fold (Tukey HSD Test, P<0.01), respectively; FIG. 4B). However, only Ezh2[FL]-dCas9 was able to deposit H3K27me3 at the HER2 promoter, producing a 9-fold enrichment compared to dCas9 with no effector domain (Tukey HSD Test, P<0.01, FIG. 4C). Therefore, similar to the case of SUV[SET]-dCas9, the data suggest that Ezh2 residues in addition to those in the CXC and SET domains are required for H3K27 trimethylation activity. Further experiments were performed to test if gene repression by Ezh2[SET]-dCas9 was associated with other known repressive histone marks. There was no increase in H3K9me2 or H3K9me3 that could explain the repression caused by Ezh2[SET]-dCas9 (for which H3K27me3 was expected but not observed) (FIG. 4D). The lack of deposition of expected or alternative repressive histone marks again supported the conclusion that repression by Ezh2[SET]-dCas9 does not require histone methylation.

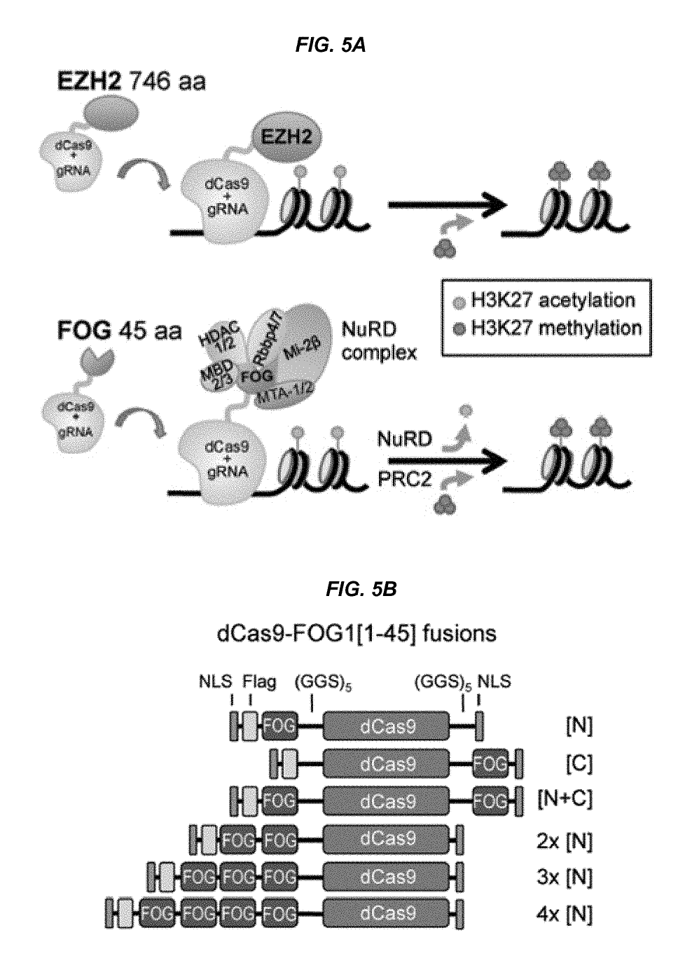

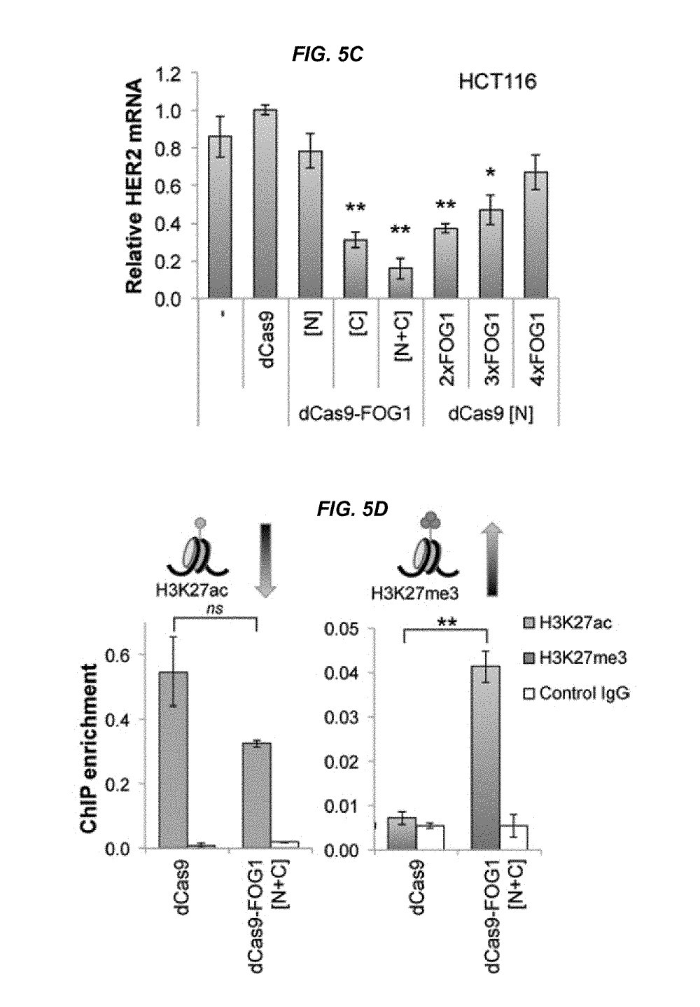

Taken together, these results supported a hypothesis that neither H3K9me3 nor H3K27me3 must precede or are causative for repression. A possible non-epigenetic mechanism for repression was the simple steric interference of endogenous regulatory components by the binding of the dCas9-ED fusions. dCas9 alone did not cause repression by this mechanism, as cells transfected with only an mCherry expression plasmid displayed HER expression at a level similar to a dCas9 with no ED (FIG. 5C). However, the repression displayed by the dCas9-ED fusions above suggested that these dCas9 appendages might produce interference. This non-catalytic mechanism was investigated using catalytic mutants of the Ezh2[SET] domain. Catalytic sites for Ezh2 SET had been identified and defined by their ability to contact and methylate H3K27, including invariant residues involved in targeting lysine or S-adenosyl methionine (30, 47). Therefore, tyrosine 641 was mutated to alanine (Y641A) and tyrosine 726 was mutated to a phenylalanine (Y726F), creating Ezh2[SET-Y641A]-dCas9 and Ezh2[SET-Y726F]-dCas9, respectively (FIG. 4E). If repressive activity is truly uncoupled from epigenetic writing activity, the mutant fusions would be expected to repress gene expression similarly to the catalytically active Ezh2[SET]. Indeed both, Ezh2[SET-Y641A]-dCas9 and Ezh2[SET-Y726F]-dCas9 repressed HER2 expression similar to the wild-type Ezh2[SET]-dCas9 fusion (FIG. 4F). These data strongly suggest that some or all of the repression observed using these dCas9-ED fusions could be due to non-catalytic mechanisms such as steric interference. However, since dCas9-G9A[SET] and Ezh2[FL]-dCas9 did clearly deposit their expected epigenetic mark, these latter data also reinforce that neither H3K9me3 nor H3K27me3 must precede or are causative for repression.

dCas9-FOG1[1-45] is a Novel and Efficient Transcriptional Repressor Producing H3K27 Trimethylation

As an alternative to the “direct tethering” of the H3K27me3 methyltransferase Ezh2 (FIG. 5A, top), a “recruitment” paradigm was examined in which an endogenous modifying complex could be recruited by a small peptide attached to dCas9 (FIG. 5A, bottom). Recruitment is also the strategy used more frequently by natural transcription factors rather than the direct tethering of enzymes. One such small peptide, the N-terminal 45 residues of Friend of GATA1 (FOG1), has been associated with the trimethylation of H3K27. It had previously been shown that repression by the transcription factors GATA1 and GATA2 is dependent on a small conserved domain at the N-terminus of FOG1, which in turn can bind directly to the nucleosome remodeling and deacetylase (NuRD) complex (31). Recruitment of the NuRD complex causes histone deacetylation at GATA1/2 target sites, followed by recruitment of the Polycomb Repressive Complex 2 (PRC2) responsible for methylation of H3K27 (32) (FIG. 5A, bottom). However, FOG1 had not previously been used with any of the programmable DNA-binding platforms (e.g., ZFPs, TALEs, or dCas9). FOG1[1-45] (SEQ ID NO:3) was fused to the N-terminus, the C-terminus, or to both the N- and C-termini of dCas9 (FIG. 5B). In contrast to the results observed for G9A[SET], SUV[SET], and Ezh2[SET], the FOG1[1-45]-dCas9 fusion at the N-terminus did not give rise to a significant decrease in HER2 transcription in HCT116 cells. However, the C-terminal dCas9-FOG1[1-45] repressed HER2 expression 3.2-fold (Tukey HSD test, P=0.004; FIG. 5C). In further contrast, the strongest repression was observed with dCas9 containing FOG1[1-45] fusions on both the N- and the C-termini (6.2-fold; Tukey HSD test, P=0.001; FIG. 5C). To evaluate possible synergistic activity of multiple FOG1[1-45] effectors, N-terminal dCas9 fusions were created with arrays of two, three or four FOG1[1-45] repeats separated by 15-amino acid linkers (i.e., (GGS)₅ (SEQ ID NO:75)). However, these arrays failed to repress as effectively as two FOG1[1-45] domains on either terminus, perhaps due to their reduced expression levels compared to the other FOG1-containing proteins (FIG. 3B).

Since FOG1[1-45]-dCas9-FOG1[1-45] (also referred to herein as dCas9-FOG1 [N+C]) showed the strongest repression at the HER2 target locus, ChIP-qPCR assays were performed to determine enrichment of the histone marks H3K27ac and H3K27me3. While the effect on H3K27ac was not significant (Tukey test, P=0.07), H3K27me3 was increased 5.8-fold (Tukey test, P<0.01; FIG. 5D). These data demonstrate that targeting FOG1[1-45] to a specific site in the genome was sufficient to cause H3K27 trimethylation. Taken together, these findings identify FOG1[1-45]-dCas9-FOG1[1-45] as a novel transcriptional repressor that is associated with H3K27 trimethylation.

### A Toolbox of Targetable Epigenetic Regulators Demonstrate Variable Levels of Repression at Three Loci in Two Cell Types.

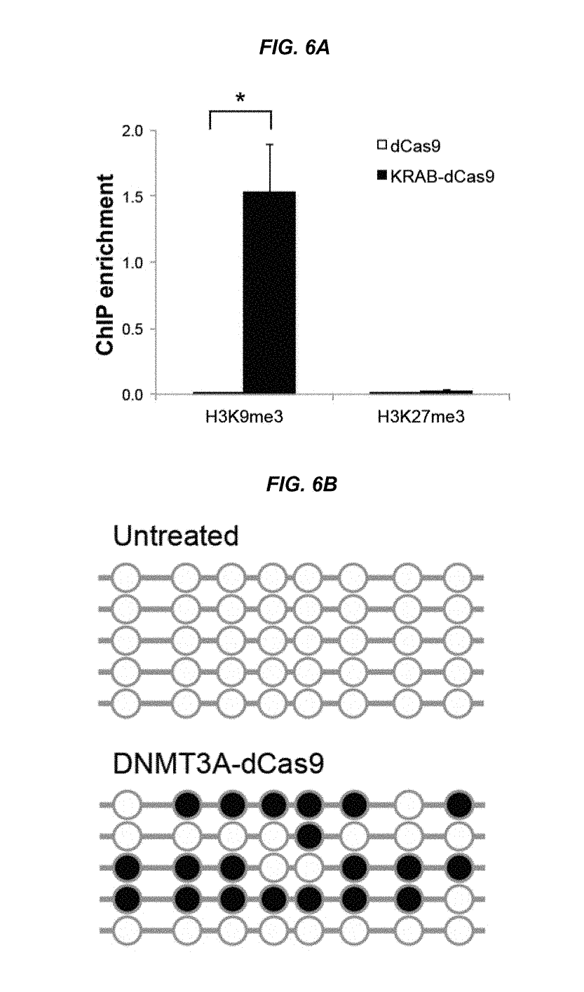

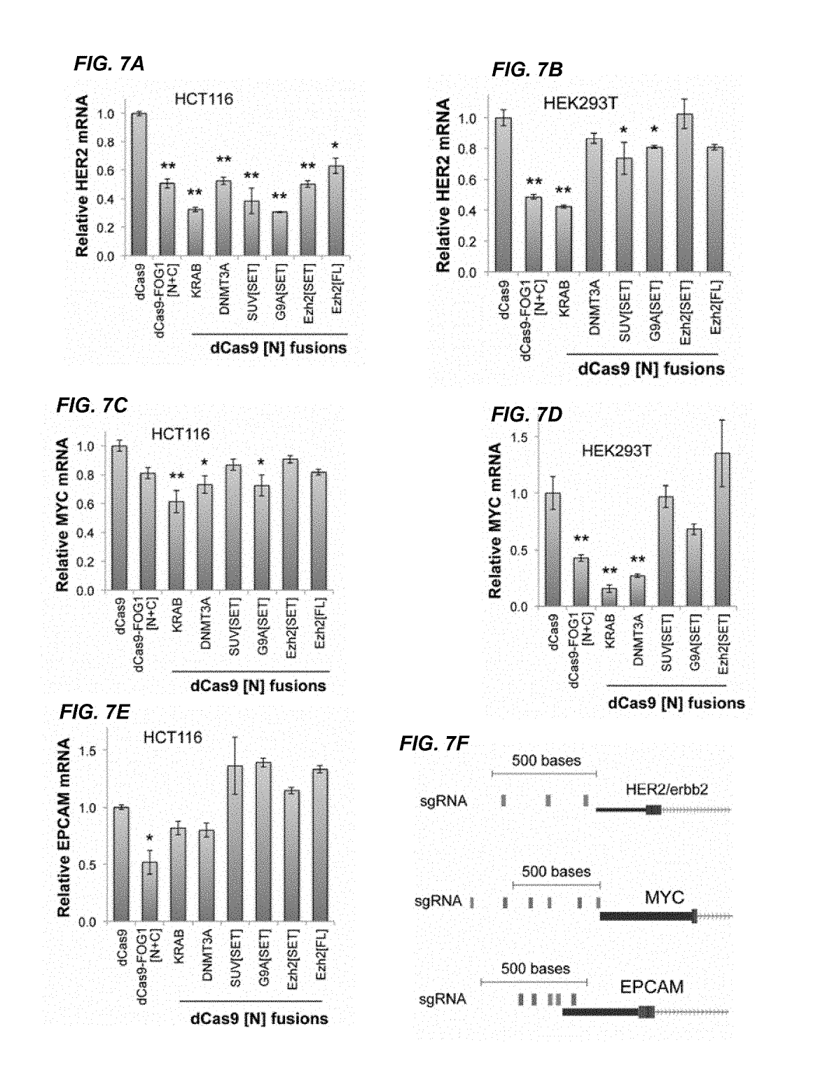

The effect of targeted epigenetic reprogramming is influenced by factors such as epigenetic marks, three-dimensional interactions (e.g., between a promoter and an enhancer, or localization of the DNA region to a subnuclear compartment such as a transcriptional factory), and initial expression levels, which in some instances are locus- and cell-type dependent. Therefore, seven epigenetic modifiers at the HER2, MYC, and EPCAM promoters were investigated in HCT116 and HEK293T cells. To be more comprehensive in the comparison of epigenetic modifiers having a common dCas9 architecture, the additional constructs KRAB-dCas9 and DNMT3A-dCas9 were created. The Krüppel-associated box (KRAB) domain is a commonly used repression domain that, like FOG1, acts by the recruitment of chromatin-modifying complexes. The KRAB domain achieves repression in association with the recruitment of the KAP1 co-repressor complex and is associated with H3K9me3 deposition (27). The DNMT3A repression domain extended the toolbox to include targeted de novo DNA methylation (16-21). As reported in previous studies (16, 17, 22, 25, 48), KRAB-dCas9 caused trimethylation of H3K9 and DNMT3A-dCas9 induced DNA methylation at the targeted HER2 promoter (FIGS. 6A and 6B, respectively). All dCas9 fusions caused some repression of HER2 expression in HCT116 cells (Tukey HSD Test, P<0.05 and P<0.01; FIG. 7A). Ezh2[SET]-dCas9, FOG1[1-45]-dCas9-FOG1[1-45], and DNMT3A-dCas9 produced 2-fold downregulation of HER2 expression, placing them as somewhat less efficacious than KRAB-dCas9, G9A[SET]-dCas9, and SUV[SET]-dCas9. Differences in HER2 repression were not correlated with differences in the amount dCas9-fusion protein produced in cells (FIG. 3C). HER2 is actively transcribed in HCT116 and HEK293T cells and hence both contain features associated with active promoters. Hallmarks of active promoters are a DNasel hypersensitive site, acetylation marks (i.e., H3K27ac and H3K9ac), and methylation marks (i.e., H3K4me3 and H3K4me2) (49). In HEK293T cells, only FOG1[1-45]-dCas9-FOG1[1-45] and KRAB-dCas9 were able to downregulate HER2 expression (2.1-fold and 2.4-fold, respectively, FIG. 7B). These data clearly demonstrate that although both cell types have similar epigenetic profiles, epigenetic dCas9-fusions acted in a cell-type dependent manner.

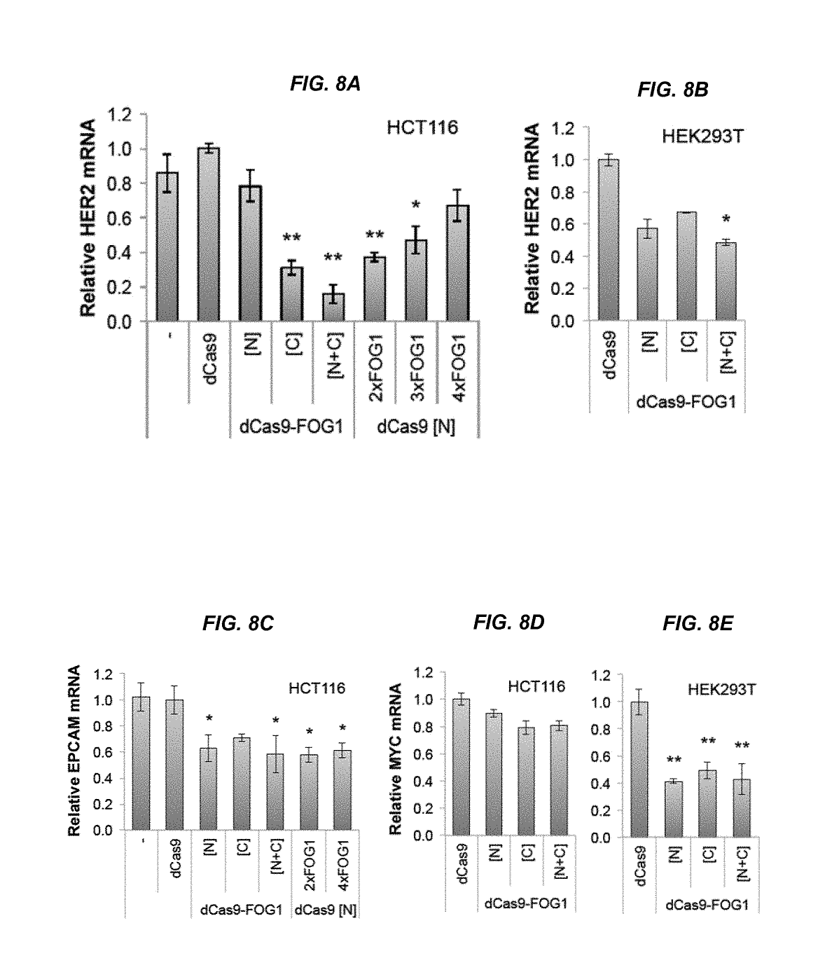

Next, dCas9 fusions were tested at different gene promoters. Very modest or no repressive activity was observed at the MYC promoter in HCT116 cells (Tukey HSD Test, P<0.05 and P<0.01; FIG. 7C), while in HEK293T cells KRAB-dCas9 caused robust downregulation of MYC expression (6.2-fold) and DNMT3A-dCas9 and FOG1[1-45]-dCas9-FOG1[1-45] repressed MYC expression 3.7-fold and 2.3-fold, respectively (Tukey HSD Test, P<0.01; FIG. 7D). No significant downregulation was observed with G9A[SET]-dCas9, SUV[SET]-dCas9 and Ezh2[SET]-dCas9. These latter effects may be due to the increased copy number of the MYC gene in this cell line. Finally, dCas9 fusions were targeted to the EPCAM promoter in HCT116 cells (FIG. 7E). Surprisingly, only FOG1[1-45]-dCas9-FOG1[1-45] showed significant downregulation (2-fold, Tukey HSD Test, P<0.05). Similar locus and cell-type differences in repression were observed for different configurations of dCas9 with FOG1[1-45] (FIG. 8). For each target, a pool of between three and six sgRNAs was used to target dCas9 fusions to the gene promoter (FIG. 7F). Taken together, these data identify FOG1[1-45]-dCas9-FOG1[1-45] and KRAB-dCas9 as the most potent transcriptional repressors at most tested target sites. It was notable that direct fusions of dCas9 with chromatin-modifying enzymes were much more susceptible to differences in cell type or target region.

Effector Fusions to the Catalytically Inactive Cpf1 (dCpf1) Are Not Effective

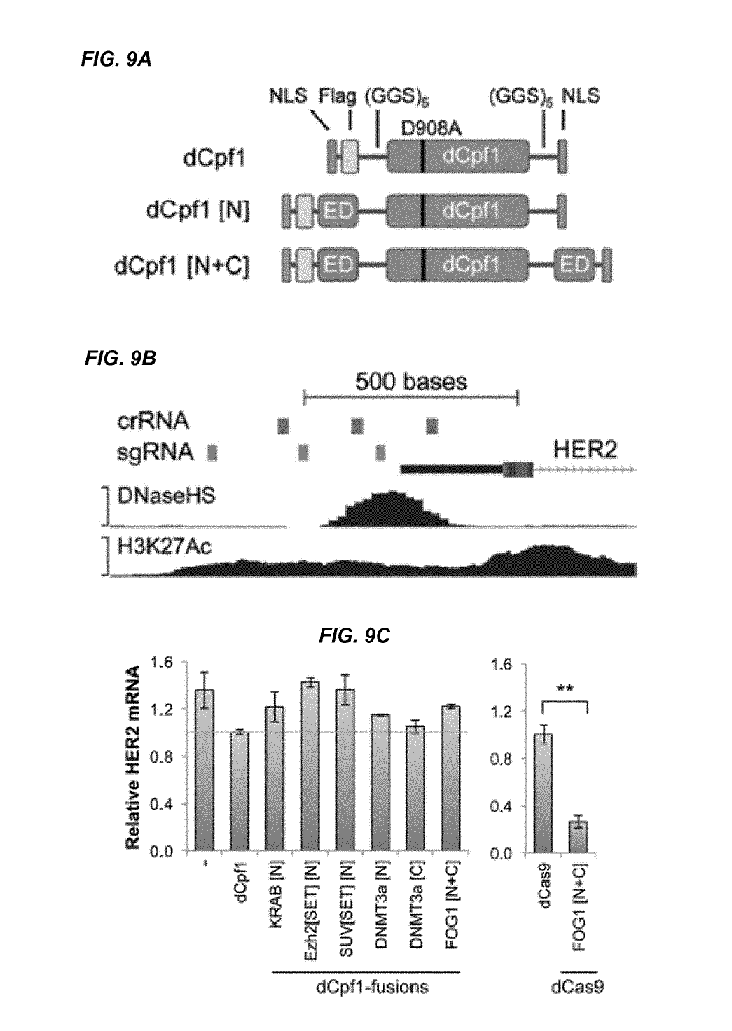

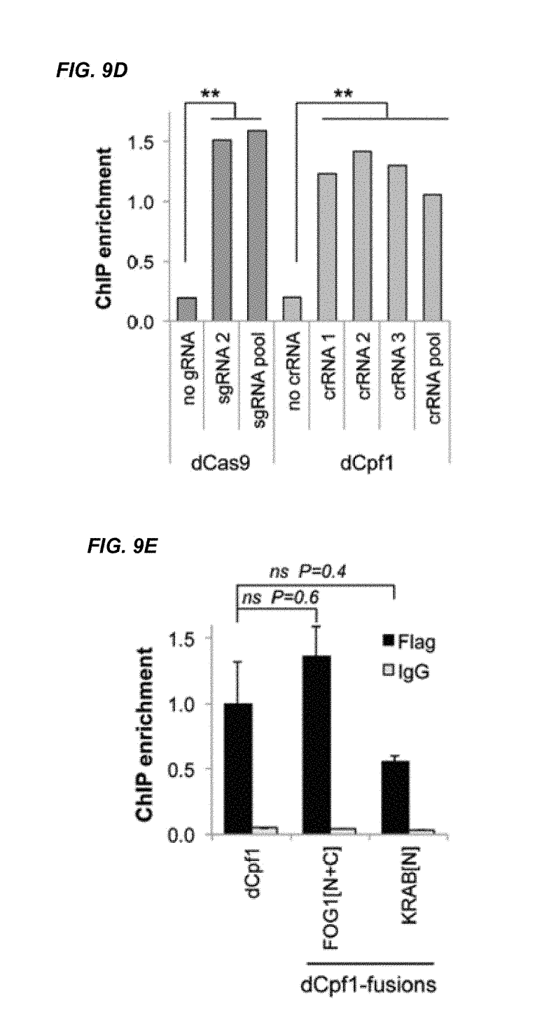

To guide different epigenetic effector domains to unique sites within the same or different regulatory elements, it is useful to employ orthogonal programmable DNA-binding platforms. The RNA-guided endonuclease Cpf1, a type V CRISPR/Cas system, offers a genome editing alternative to the type II CRISPR/Cas9 endonuclease (39, 50, 51). Unlike Cas9, for which a CRISPR targeting RNA and a trans-activating RNA are combined to form a guide RNA, Cpf1 requires only a single CRISPR gRNA (crRNA). Acidaminococcus (As)Cpf1 efficiently cleaves target DNA adjacent to a short T-rich PAM recognition site (5′-TTTN-3′) whereas Streptococcus pyogenes (Sp)Cas9 requires a G-rich PAM site (5′-NGG-3′), hence broadening in principle the number and diversity of target sites in the genome that are accessible to precise gene editing. Since the goal is to develop tools that target the epigenome, but do not cleave the target DNA, a catalytically “dead” Cpf1 [D908A] (dCpf1; FIG. 9A) was used. To confirm loss of cleavage activity of dCpf1, single strand annealing (SSA) assays were performed using an mCherry reporter system (52). The mCherry gene was split into two inactive fragments containing overlapping homologies with a HER2 promoter target site between them (FIG. 1A). In cells, cleavage at the HER2 site initiates single strand annealing and generates an active mCherry gene, causing cells to accumulate fluorescent mCherry protein. Co-transfection of wild type AsCpf1 with a HER2 crRNA resulted in red fluorescence; however, as expected, no red fluorescence was observed when catalytically inactive dCpf1 was used (FIG. 1B). Subsequently, KRAB-dCpf1, EZH2[SET]-dCpf1, SUV[SET]-dCpf1, DNMT3A-dCpf1, dCpf1-DNMT3A, and FOG1[1-45]-dCpf1-FOG1[1-45] (FIG. 9A) were constructed and their repressive activity was tested at the HER2 promoter in HCT116 cells using three crRNAs simultaneously (FIG. 9B). Surprisingly, none of the dCpf1 fusions were able to repress transcription of HER2, while a dCas9 version of FOG1[1-45]-dCas9-FOG1[1-45] demonstrated the expected repression (FIG. 9C). ChIP assays were then performed to confirm that creating the catalytic mutant dCpf1 did not interfere with the ability of dCpf1 to bind to its target site. dCas9 binding to the HER2 promoter was used as the gold standard and was targeted to the HER2 promoter either by one sgRNA (sgRNA2) or a pool of threes (FIG. 2B). Similarly, dCpf1 was targeted to the HER2 promoter with each individual crRNA or a pool of all three crRNAs (FIG. 9B). ChIP enrichments of dCas9 or dCpf1 were indistinguishable whether one or a pool of sgRNAs or crRNAs were used (Tukey HSD Test, P=0.001, FIG. 9D). After this important preliminary finding, it was next assessed whether the addition of effector domains destabilized dCpf1 binding to the target site. ChIP enrichment was assessed for FOG1[1-45]-dCpf1-FOG1[1-45] and KRAB-dCpf1. Binding of FOG1[1-45]-dCpf1-FOG1[1-45] and KRAB-dCpf1 were not significantly different when compared to dCpf1 alone (Tukey HSD Test, FIG. 9E). These data suggest major differences between the dCas9 and dCpf1 scaffolds and mode of action when bound to the target site.

EZH2[FL]-dCas9 and DNMT3A-dCas9 Establish Persistent Repression, while FOG1[1-45]-dCas9-FOG1[1-45] and KRAB-dCas9 Drive Robust Transient Repression

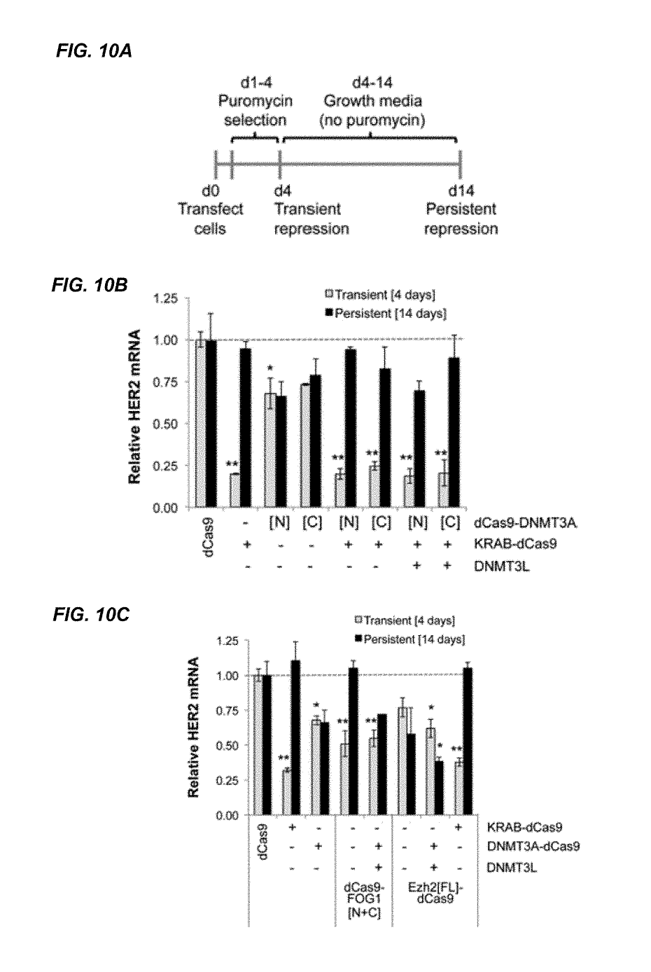

Next, it was tested whether transient expression of dCas9 fusion proteins could cause persistent HER2 gene repression and if combinations of dCas9 fusion proteins could increase transient and/or persistent downregulation of HER2 expression. Transient repression was measured four days after transfection under puromycin selection to enrich for transfected cells, while the persistent effect was determined after cells were grown for an additional ten days in puromycin-free media (FIG. 10A). This procedure enriched for transfected cells but avoided selection of stably integrated epigenetic modifier expression plasmids, ensuring that persistent repression would be due to altered epigenetic states. Repressive activity was determined for DNMT3A fused to the N- or C-terminus of dCas9 (DNMT3A-dCas9 and dCas9-DNMT3A, respectively). DNMT3A-dCas9 and dCas9-DNMT3A caused only modest downregulation of 1.5-fold and 1.4-fold, respectively; however, the repression was persistent over 10 days (FIG. 10B). In contrast, KRAB-dCas9 achieved a 5-fold downregulation of HER2, but expression was completely restored 10 days later. KRAB-dCas9 dominated transient repression, and addition of DNMT3A-dCas9, dCas9-DNMT3A, or overexpressed DNMT3L neither increased repression nor persistence (FIG. 10B). Two H3K27me3 producing fusions, FOG1[1-45]-dCas9-FOG1[1-45] and Ezh2[FL]-dCas9, were assessed for their effects on the level and persistence of repression. FOG1[1-45]-dCas9-FOG1[1-45] downregulated HER2 expression 2-fold, but HER2 expression reverted to normal after 10 days (FIG. 10C). Addition of DNMT3A-dCas9 and overexpression of DNMT3L improved the persistence of downregulation; however, the same expression level and persistence was achieved by DNMT3A-dCas9 alone. Ezh2[FL]-dCas9 was also able to cause a level of HER2 downregulation similar to DNMT3A-dCas9. The level and persistence of repression by Ezh2[FL]-dCas9 was further enhanced by addition of DNMT3A-dCas9 and overexpressed DNMT3L (Tukey HSD Test, P=0.02; FIG. 10C). Taken together, FOG1[1-45]-dCas9-FOG1[1-45] and KRAB-dCas9 produced a transient but strong repression, while Ezh2[FL]-dCas9 and DNMT3A-dCas9 drove persistent but more modest repression.

### Discussion

Precise control of transcription and epigenetics at a defined genomic locus provides an ability to dissect links between the two processes in a way not formerly possible. In this study, a set of epigenome editing tools was generated to deposit epigenetic marks typically associated with a repressed chromatin state, including DNA methylation and histone methylation (both H3K9me3 and H3K27me3). The epigenetic fusions of dCas9 with histone methyltransferases (HMT) described herein complement recently described epigenetic editing tools, which have been mostly focused on DNA methylation and demethylation (16-21). The present study made use of a dCas9 architecture and assayed a broad assortment of epigenetic effector domains at three loci in two cell types. Direct enzyme tethering vs. co-repressor recruitment strategies were also examined.

The major finding of this study was that transcriptional repression was independent of deposition of the expected repressive chromatin mark. While dCas9 alone did not produce repression, evidence from Ezh2[SET]-dCas9 catalytic mutants (FIG. 4F) suggested that some amount of repression was due to a non-catalytic activity of the effector domains. This activity could occur by a mechanism such as steric hindrance of endogenous activation factors, or by an interaction with other components of repression complexes. A similar observation was recently reported by Wysocka and co-workers, in which the methyltransferase catalytic activity of MII3/4 proteins was dispensable for transcription, but the proteins themselves were required due to their protein binding interactions with other factors (53). However, several of the domains tested in this study were able to deposit their expected chromatin marks, but the chromatin marks appeared to produce no additional gene repression (FIGS. 2E and 4C). These data therefore demonstrate that deposition of so-called epigenetic repressive histone marks is not sufficient to cause transcriptional repression.

The KRAB domain achieves repression in association with recruitment of the KAP1 co-repressor complex which contains the histone methyltransferase SETDB1, initiating trimethylation of H3K9 (27). The histone methyltransferases SUV39H1 and G9A have also been associated with H3K9me3. In contrast, the two new functional domains introduced in this study, Ezh2 and FOG1, are both associated with H3K27me3. Ezh2 is a catalytic component of the PRC2 complex responsible for H3K27me2/3. GATA-1 and its cofactor Friend of GATA-1 (FOG1) bind to their genomic targets and repress gene expression through recruitment of the nucleosome remodeling deacetylase (NuRD). In biochemical studies, FOG1[1-45] has been shown to interact with several proteins that are part of the NuRD complex, such as histone deacetylases HDAC1/2, CHD4, MBD2/3 as well as MTA-1 and MTA-2 (31, 54). NuRD-mediated deacetylation of H3K27 in turn allows for H3K27 trimethylation by the PRC2 complex (32-55). In the studies described herein, FOG1[1-45]-dCas9-FOG1[1-45] showed the strongest repression at the HER2 target locus compared to any of the other effector domains tested, and also provided strong deposition of H3K27me3. These findings present FOG1[1-45]-dCas9-FOG1[1-45] as a newly described, highly efficient transcriptional repressor associated with H3K27 trimethylation.

The catalytic domains for Ezh2, G9A and SUV39H1 have been mapped to their C-terminal SET domains (30, 47). G9A[SET]-dCas9 was able to deposit H3K9me3 and a full-length Ezh2[FL]-dCas9 was able to deposit H3K27me3; however SUV[SET]-dCas9 and Ezh2[SET]-dCas9 were not able to deposit their expected marks. These observations indicate that the SET domains of SUV and Ezh2 are not sufficient for H3K9 or H3K27 trimethylation but that other parts of the full-length proteins may also be required for histone methylation, at least in the context of dCas9 fusion proteins. Perhaps this is not unexpected as other domains of the Ezh2 protein are important for interaction with members of the PRC2 complex, such as Suz12 and EED, as well as other epigenetic modifying enzymes such as DNA methyltransferases (56, 57). It should also be noted that SUV39H1 has Glu-repeat, Cys-repeat, Ankyrin, and Chomodomain domains upstream of the SET domain (30), which may be important for catalytic (epigenetic writing) activity.

Two strategies can be used to epigenetically repress a specific endogenous gene: 1) direct targeting of a chromatin modifying enzyme itself to DNA or 2) recruitment of a chromatin remodeling complex that contains several enzymatic capabilities. Although in nature, epigenetic enzymes are rarely attached to DNA-binding domains directly, the results presented here using the enzymatic domains of EZH2, SUV, and G9A, as well as those of several other studies (16, 17, 22-24), suggest that the first strategy can be effective experimentally. The novel transcriptional repressor consisting of dCas9 fused to FOG1[1-45] is an example of the alternative repression strategy based on recruitment of a co-repressor, as opposed to fusion of an enzymatic component to dCas9. In addition to any functional advantages (e.g., improved target-gene repression), the use of a short peptide is less likely to interfere with endogenous regulatory factors at the promoter than the direct tethering of large enzymes. It also provides an opportunity to increase its effect by multiplexing the short interaction peptides, such as is frequently done with the herpes simplex VP16 activation domain to produce the more effective VP64 (58, 59). However, the data demonstrate that some configurations of arrayed repeats can actually reduce protein expression, which could have accounted for the reduced repression of the tandem FOG1 arrays.

The toolbox of epigenetic editors described herein was found to have locus- and cell-type dependent effects on transcriptional repression, ranging from nearly no significant repression by any factor at the MYC promoter in HCT116 cells to nearly 10-fold repression by one factor at MYC in HEK293T cells. HCT116 is a colon cancer cell line that contains amplified regions in the genome resulting in additional copies of affected genes. The MYC gene is located in such an amplified region in HCT116 cells and is thus present in three copies, while there are two copies of the EPCAM and HER2 genes. It cannot be concluded whether the lack of repression is a cell-type specific phenomenon, per se, or if it is more difficult to achieve repression in the presence of additional MYC gene copies. The effect of targeted epigenetic reprograming might also be influenced by existing epigenetic marks, three-dimensional interactions, and initial expression levels, as well as other factors

Surprisingly, none of the dCpf1-effector domain fusions had an effect on gene expression, despite evidence of binding to the DNA target sites. In contrast to the G-rich PAM site (5′-NGG-3′) required by the Streptococcus pyogenes (Sp)Cas9, Acidaminococcus sp. BV3L6 (As)Cpf1 is an RNA-guided nuclease that can use a short T-rich PAM recognition site (5′-TTTN-3′) (39, 50). Targeting both T-rich as well as C-rich chromatin regions would broaden the number of target sites in the genome that are accessible to epigenetic editing, and would have been a useful orthogonal platform for targeting different effectors to the same gene or simultaneously activating and repressing different genes in the same cell. Notably, there have not been any reports of dCpf1 based activators (e.g., VP64) or repressors (e.g., KRAB) in mammalian cells. In Arabidopsis, fusions of catalytic inactive Cpf1 (AsCpf1[D908A] and LbCpf1 [D832A]) with three copies of the SRDX repressor domain were used to repress a noncoding RNA (60). Unfortunately, the dCpf1 used here was not suitable for targeted transcriptional regulation. It is also noted that Ezh2[SET]-dCas9 was observed to produce gene repression through a non-catalytic process such as steric hindrance (FIG. 4F), but no such repression was observed when Ezh2[SET] was tethered to dCpf1. These observations suggest unexpected differences between dCas9 and dCpf1 platforms. However, it is possible that dCpf1 fusions will be successful with different features or at different genomic loci.

In addition to orthogonal gene regulation, epigenetic editing is useful for effecting persistent changes in gene expression without altering genetic sequence. In nature, H3K9me3 and H3K27me3 are often associated with silenced states of genes and other elements that are stable over the lifetime of an individual. However, far less is known about the transitions between active and silenced states. It has been shown that targeting DNMT3A to a gene promoter can be sufficient to achieve persistent gene silencing (16, 61, 62). Although targeting DNMT3A results in methylation at the target site, it has been found that the downregulation of gene expression is often modest (17, 48). In certain cell types, targeting KRAB and DNMT3L in addition to DNMT3A was required for persistent gene silencing (61). However, KRAB-dCas9 had no effect on promoting persistent silencing in the present study, while the dCas9 fusion with the epigenetic writer of H3K27me3 (Ezh2[FL]) facilitated persistence.

Targeting epigenetic modifying enzymes allowed for the interrogation of the causal relationship between the epigenetic marks and gene expression at the target site. Surprisingly, it was found that deposition of the expected histone modification was not sufficient for transcriptional repression. This result was similar to a previous finding that the level of H3K27ac at an enhancer region was not correlated with the activity of that enhancer in its endogenous genomic context (63). The present study has expanded the list of tools available for epigenetic editing (6) to include new targeted tools to deposit H3K27me3. However, almost all targeted epigenetic modifiers reported to date have fallen well short of producing the dramatic differences in the level of gene repression observed in natural epigenetic states.

It is understood that the examples and embodiments described herein are for illustrative purposes only and that various modifications or changes in light thereof will be suggested to persons skilled in the art and are to be included within the spirit and purview of this application and scope of the appended claims. All publications, patents, patent applications, and sequence accession numbers cited herein are hereby incorporated by reference in their entirety for all purposes.

### X. REFERENCES

1. Jenuwein, T. and Allis, C. D. (2001) Translating the histone code. Science, 293, 1074-1080.
2. Berger, S. L. (2007) The complex language of chromatin regulation during transcription. Nature, 447, 407-412.
3. Consortium, E. P., Bernstein, B. E., Birney, E., Dunham, I., Green, E. D., Gunter, C. and Snyder, M. (2012) An integrated encyclopedia of DNA elements in the human genome. Nature, 489, 57-74.
4. Roadmap Epigenomics, C., Kundaje, A., Meuleman, W., Ernst, J., Bilenky, M., Yen, A., Heravi-Moussavi, A., Kheradpour, P., Zhang, Z., Wang, J. et al. (2015) Integrative analysis of 111 reference human epigenomes. Nature, 518, 317-330.
5. You, J. S., Kelly, T. K., De Carvalho, D. D., Taberlay, P. C., Liang, G. and Jones, P. A. (2011) OCT4 establishes and maintains nucleosome-depleted regions that provide additional layers of epigenetic regulation of its target genes. Proceedings of the National Academy of Sciences of the United States of America, 108, 14497-14502.
6. Stricker, S. H., Koferle, A. and Beck, S. (2017) From profiles to function in epigenomics. Nat Rev Genet, 18, 51-66.
7. Segal, D. J. and Meckler, J. F. (2013) Genome engineering at the dawn of the golden age. Annu Rev Genomics Hum Genet, 14, 135-158.
8. Falahi, F., Sgro, A. and Blancafort, P. (2015) Epigenome engineering in cancer: fairytale or a realistic path to the clinic? Frontiers in oncology, 5, 22.
9. Hilton, I. B. and Gersbach, C. A. (2015) Enabling functional genomics with genome engineering. Genome research, 25, 1442-1455.
10. Jinek, M., Chylinski, K., Fonfara, I., Hauer, M., Doudna, J. A. and Charpentier, E. (2012) A programmable dual-RNA-guided DNA endonuclease in adaptive bacterial immunity. Science, 337, 816-821.
11. Jinek, M. (2012) A programmable dual-RNA-guided DNA endonuclease in adaptive bacterial immunity. Science, 337, 816-821.
12. Sternberg, S. H., Redding, S., Jinek, M., Greene, E. C. and Doudna, J. A. (2014) DNA interrogation by the CRISPR RNA-guided endonuclease Cas9. Nature, 507, 62-67.
13. Perez-Pinera, P., Kocak, D. D., Vockley, C. M., Adler, A. F., Kabadi, A. M., Polstein, L. R., Thakore, P. I., Glass, K. A., Ousterout, D. G., Leong, K. W. et al. (2013) RNA-guided gene activation by CRISPR-Cas9-based transcription factors. Nature methods, 10, 973-976.
14. Maeder, M. L., Linder, S. J., Cascio, V. M., Fu, Y., Ho, Q. H. and Joung, J. K. (2013) CRISPR RNA-guided activation of endogenous human genes. Nature methods, 10, 977-979.
15. Gilbert, L. A., Larson, M. H., Morsut, L., Liu, Z., Brar, G. A., Torres, S. E., Stern-Ginossar, N., Brandman, O., Whitehead, E. H., Doudna, J. A. et al. (2013) CRISPR-mediated modular RNA-guided regulation of transcription in eukaryotes. Cell, 154, 442-451.
16. Vojta, A., Dobrinic, P., Tadic, V., Bockor, L., Korac, P., Julg, B., Klasic, M. and Zoldos, V. (2016) Repurposing the CRISPR-Cas9 system for targeted DNA methylation. Nucleic acids research, 44, 5615-5628.
17. McDonald, J. I., Celik, H., Rois, L. E., Fishberger, G., Fowler, T., Rees, R., Kramer, A., Martens, A., Edwards, J. R. and Challen, G. A. (2016) Reprogrammable CRISPR/Cas9-based system for inducing site-specific DNA methylation. Biology open, 5, 866-874.
18. Xu, X., Tao, Y., Gao, X., Zhang, L., Li, X., Zou, W., Ruan, K., Wang, F., Xu, G. L. and Hu, R. (2016) A CRISPR-based approach for targeted DNA demethylation. Cell discovery, 2, 16009.
19. Choudhury, S. R., Cui, Y., Lubecka, K., Stefanska, B. and Irudayaraj, J. (2016) CRISPR-dCas9 mediated TET1 targeting for selective DNA demethylation at BRCA1 promoter. Oncotarget.
20. Morita, S., Noguchi, H., Horii, T., Nakabayashi, K., Kimura, M., Okamura, K., Sakai, A., Nakashima, H., Hata, K., Nakashima, K. et al. (2016) Targeted DNA demethylation in vivo using dCas9-peptide repeat and scFv-TET1 catalytic domain fusions. Nature biotechnology, 34, 1060-1065.
21. Liu, X. S., Wu, H., Ji, X., Stelzer, Y., Wu, X., Czauderna, S., Shu, J., Dadon, D., Young, R. A. and Jaenisch, R. (2016) Editing DNA Methylation in the Mammalian Genome. Cell, 167, 233-247 e217.
22. Kearns, N. A., Pham, H., Tabak, B., Genga, R. M., Silverstein, N. J., Garber, M. and Maehr, R. (2015) Functional annotation of native enhancers with a Cas9-histone demethylase fusion. Nature methods, 12, 401-403.
23. Hilton, I. B., D'Ippolito, A. M., Vockley, C. M., Thakore, P. I., Crawford, G. E., Reddy, T. E. and Gersbach, C. A. (2015) Epigenome editing by a CRISPR-Cas9-based acetyltransferase activates genes from promoters and enhancers. Nature biotechnology, 33, 510-517.
24. Cano-Rodriguez, D., Gjaltema, R. A., Jilderda, L. J., Jellema, P., Dokter-Fokkens, J., Ruiters, M. H. and Rots, M. G. (2016) Writing of H3K4Me3 overcomes epigenetic silencing in a sustained but context-dependent manner. Nature communications, 7, 12284.
25. Thakore, P. I., D'Ippolito, A. M., Song, L., Safi, A., Shivakumar, N. K., Kabadi, A. M., Reddy, T. E., Crawford, G. E. and Gersbach, C. A. (2015) Highly specific epigenome editing by CRISPR-Cas9 repressors for silencing of distal regulatory elements. Nature methods, 12, 1143-1149.
26. Schultz, D. C., Ayyanathan, K., Negorev, D., Maul, G. G. and Rauscher, F. J., 3rd. (2002) SETDB1: a novel KAP-1-associated histone H3, lysine 9-specific methyltransferase that contributes to HP1-mediated silencing of euchromatic genes by KRAB zinc-finger proteins. Genes & development, 16, 919-932.
27. Feschotte, C. and Gilbert, C. (2012) Endogenous viruses: insights into viral evolution and impact on host biology. Nat Rev Genet, 13, 283-296.
28. Schultz, D. C., Friedman, J. R. and Rauscher, F. J., 3rd. (2001) Targeting histone deacetylase complexes via KRAB-zinc finger proteins: the PHD and bromodomains of KAP-1 form a cooperative unit that recruits a novel isoform of the Mi-2alpha subunit of NuRD. Genes & development, 15, 428-443.
29. Falahi, F., Huisman, C., Kazemier, H. G., van der Vlies, P., Kok, K., Hospers, G. A. and Rots, M. G. (2013) Towards sustained silencing of HER2/neu in cancer by epigenetic editing. Molecular cancer research: MCR, 11, 1029-1039.
30. Dillon, S. C., Zhang, X., Trievel, R. C. and Cheng, X. (2005) The SET-domain protein superfamily: protein lysine methyltransferases. Genome biology, 6, 227.
31. Hong, W., Nakazawa, M., Chen, Y. Y., Kori, R., Vakoc, C. R., Rakowski, C. and Blobel, G. A. (2005) FOG-1 recruits the NuRD repressor complex to mediate transcriptional repression by GATA-1. The EMBO journal, 24, 2367-2378.
32. Ross, J., Mavoungou, L., Bresnick, E. H. and Milot, E. (2012) GATA-1 utilizes Ikaros and polycomb repressive complex 2 to suppress Hes1 and to promote erythropoiesis. Molecular and cellular biology, 32, 3624-3638.
33. O'Geen, H., Henry, I. M., Bhakta, M. S., Meckler, J. F. and Segal, D. J. (2015) A genome-wide analysis of Cas9 binding specificity using ChIP-seq and targeted sequence capture. Nucleic acids research, 43, 3389-3404.
34. Rivenbark, A. G., Stolzenburg, S., Beltran, A. S., Yuan, X., Rots, M. G., Strahl, B. D. and Blancafort, P. (2012) Epigenetic reprogramming of cancer cells via targeted DNA methylation. Epigenetics, 7, 350-360.
35. Chedin, F., Lieber, M. R. and Hsieh, C. L. (2002) The DNA methyltransferase-like protein DNMT3L stimulates de novo methylation by Dnmt3a. Proceedings of the National Academy of Sciences of the United States of America, 99, 16916-16921.
36. Mali, P., Yang, L., Esvelt, K. M., Aach, J., Guell, M., DiCarlo, J. E., Norville, J. E. and Church, G. M. (2013) RNA-guided human genome engineering via Cas9. Science, 339, 823-826.
37. Montague, T. G., Cruz, J. M., Gagnon, J. A., Church, G. M. and Valen, E. (2014) CHOPCHOP: a CRISPR/Cas9 and TALEN web tool for genome editing. Nucleic Acids Res, 42, W401-407.
38. Yamano, T., Nishimasu, H., Zetsche, B., Hirano, H., Slaymaker, I. M., Li, Y., Fedorova, I., Nakane, T., Makarova, K. S., Koonin, E. V. et al. (2016) Crystal Structure of Cpf1 in Complex with Guide RNA and Target DNA. Cell, 165, 949-962.
39. Zetsche, B., Gootenberg, J. S., Abudayyeh, O. O., Slaymaker, I. M., Makarova, K. S., Essletzbichler, P., Volz, S. E., Joung, J., van der Oost, J., Regev, A. et al. (2015) Cpf1 is a single RNA-guided endonuclease of a class 2 CRISPR-Cas system. Cell, 163, 759-771.
40. Kim, D., Kim, J., Hur, J. K., Been, K. W., Yoon, S. H. and Kim, J. S. (2016) Genome-wide analysis reveals specificities of Cpf1 endonucleases in human cells. Nature biotechnology, 34, 863-868.
41. O'Geen, H., Frietze, S. and Farnham, P. J. (2010) Using ChIP-seq technology to identify targets of zinc finger transcription factors. Methods Mol Biol, 649, 437-455.
42. Li, L. C. and Dahiya, R. (2002) MethPrimer: designing primers for methylation PCRs. Bioinformatics, 18, 1427-1431.
43. Ren, C., Xu, K., Liu, Z., Shen, J., Han, F., Chen, Z. and Zhang, Z. (2015) Dual-reporter surrogate systems for efficient enrichment of genetically modified cells. Cellular and molecular life sciences: CMLS, 72, 2763-2772.
44. Anders, C., Niewoehner, O., Duerst, A. and Jinek, M. (2014) Structural basis of PAM-dependent target DNA recognition by the Cas9 endonuclease. Nature, 513, 569-573.
45. O'Geen, H., Squazzo, S. L., Iyengar, S., Blahnik, K., Rinn, J. L., Chang, H. Y., Green, R. and Farnham, P. J. (2007) Genome-wide analysis of KAP1 binding suggests autoregulation of KRAB-ZNFs. PLoS Genet, 3, e89.
46. Jamieson, K., Wiles, E. T., McNaught, K. J., Sidoli, S., Leggett, N., Shao, Y., Garcia, B. A. and Selker, E. U. (2016) Loss of HP1 causes depletion of H3K27me3 from facultative heterochromatin and gain of H3K27me2 at constitutive heterochromatin. Genome research, 26, 97-107.
47. Trievel, R. C., Beach, B. M., Dirk, L. M., Houtz, R. L. and Hurley, J. H. (2002) Structure and catalytic mechanism of a SET domain protein methyltransferase. Cell, 111, 91-103.
48. Stepper, P., Kungulovski, G., Jurkowska, R. Z., Chandra, T., Krueger, F., Reinhardt, R., Reik, W., Jeltsch, A. and Jurkowski, T. P. (2016) Efficient targeted DNA methylation with chimeric dCas9-Dnmt3a-Dnmt3L methyltransferase. Nucleic acids research.
49. Consortium, E. P. (2012) An integrated encyclopedia of DNA elements in the human genome. Nature, 489, 57-74.
50. Zetsche, B., Heidenreich, M., Mohanraju, P., Fedorova, I., Kneppers, J., DeGennaro, E. M., Winblad, N., Choudhury, S. R., Abudayyeh, O.O., Gootenberg, J. S. et al. (2016) Multiplex gene editing by CRISPR-Cpf1 using a single crRNA array. Nature biotechnology.
51. Kim, H. K., Song, M., Lee, J., Menon, A. V., Jung, S., Kang, Y. M., Choi, J. W., Woo, E., Koh, H. C., Nam, J. W. et al. (2017) In vivo high-throughput profiling of CRISPR-Cpf1 activity. Nature methods, 14, 153-159.
52. Szczepek, M., Brondani, V., Buchel, J., Serrano, L., Segal, D. J. and Cathomen, T. (2007) Structure-based redesign of the dimerization interface reduces the toxicity of zinc-finger nucleases. Nature biotechnology, 25, 786-793.
53. Dorighi, K. M., Swigut, T., Henriques, T., Bhanu, N. V., Scruggs, B. S., Nady, N., Still, C. D., 2nd, Garcia, B. A., Adelman, K. and Wysocka, J. (2017) Mll3 and Mll4 Facilitate Enhancer RNA Synthesis and Transcription from Promoters Independently of H3K4 Monomethylation. Molecular cell, 66, 568-576 e564.
54. Saathoff, H., Brofelth, M., Trinh, A., Parker, B. L., Ryan, D. P., Low, J. K., Webb, S. R., Silva, A. P., Mackay, J. P. and Shepherd, N. E. (2015) A peptide affinity reagent for isolating an intact and catalytically active multi-protein complex from mammalian cells. Bioorganic & medicinal chemistry, 23, 960-965.
55. Reynolds, N., Salmon-Divon, M., Dvinge, H., Hynes-Allen, A., Balasooriya, G., Leaford, D., Behrens, A., Bertone, P. and Hendrich, B. (2012) NuRD-mediated deacetylation of H3K27 facilitates recruitment of Polycomb Repressive Complex 2 to direct gene repression. The EMBO journal, 31, 593-605.
56. Rush, M., Appanah, R., Lee, S., Lam, L. L., Goyal, P. and Lorincz, M. C. (2009) Targeting of EZH2 to a defined genomic site is sufficient for recruitment of Dnmt3a but not de novo DNA methylation. Epigenetics, 4, 404-414.
57. Margueron, R., Justin, N., Ohno, K., Sharpe, M. L., Son, J., Drury, W. J., 3rd, Voigt, P., Martin, S. R., Taylor, W. R., De Marco, V. et al. (2009) Role of the polycomb protein EED in the propagation of repressive histone marks. Nature, 461, 762-767.
58. Cheng, A. W., Wang, H., Yang, H., Shi, L., Katz, Y., Theunissen, T. W., Rangarajan, S., Shivalila, C. S., Dadon, D. B. and Jaenisch, R. (2013) Multiplexed activation of endogenous genes by CRISPR-on, an RNA-guided transcriptional activator system. Cell research, 23, 1163-1171.
59. Beerli, R. R., Segal, D. J., Dreier, B. and Barbas III, C. F. (1998) Toward controlling gene expression at will: specific regulation of the erbB-2/HER-2 promoter by using polydactyl zinc finger proteins constructed from modular building blocks. Proceedings of the National Academy of Sciences of the United States of America, 95, 14628-14633.
60. Tang, X., Lowder, L. G., Zhang, T., Malzahn, A. A., Zheng, X., Voytas, D. F., Zhong, Z., Chen, Y., Ren, Q., Li, Q. et al. (2017) A CRISPR-Cpf1 system for efficient genome editing and transcriptional repression in plants. Nature plants, 3, 17018.
61. Amabile, A., Migliara, A., Capasso, P., Biffi, M., Cittaro, D., Naldini, L. and Lombardo, A. (2016) Inheritable Silencing of Endogenous Genes by Hit-and-Run Targeted Epigenetic Editing. Cell, 167, 219-232 e214.
62. Bintu, L., Yong, J., Antebi, Y. E., McCue, K., Kazuki, Y., Uno, N., Oshimura, M. and Elowitz, M. B. (2016) Dynamics of epigenetic regulation at the single-cell level. Science, 351, 720-724.
63. Tak, Y. G., Hung, Y., Yao, L., Grimmer, M. R., Do, A., Bhakta, M. S., O'Geen, H., Segal, D. J. and Farnham, P. J. (2016) Effects on the transcriptome upon deletion of a distal element cannot be predicted by the size of the H3K27Ac peak in human cells. Nucleic acids research, 44, 4123-4133.

### INFORMAL SEQUENCE LISTING

| SEQ | | |
|---|---|---|
| ID | | |
| NO: | Sequence | Description |
| 1 | MGQTGKKSEKGPVCWRKRVKSEYMRLRQLKRFRRADEVKTMFSSNR | Ezh2[FL] (amino acids |
| | QKILERTETLNQEWKQRRIQPVHIMTSVSSLRGTRECSVTSDLDFPAQV | 1-746 of NP_031997.2) |
| | IPLKTLNAVASVPIMYSWSPLQQNFMVEDETVLHNIPYMGDEVLDQD | |
| | GTFIEELIKNYDGKVHGDRECGFINDEIFVELVNALGQYNDDDDDDDG | |
| | DDPDEREEKQKDLEDNRDDKETCPPRKFPADKIFEAISSMFPDKGTAEE | |
| | LKEKYKELTEQQLPGALPPECTPNIDGPNAKSVQREQSLHSFHTLFCRR | |
| | CFKYDCFLHPFHATPNTYKRKNTETALDNKPCGPQCYQHLEGAKEFA | |
| | AALTAERIKTPPKRPGGRRRGRLPNNSSRPSTPTISVLESKDTDSDREAG | |
| | TETGGENNDKEEEEKKDETSSSSEANSRCQTPIKMKPNIEPPENVEWSG | |
| | AEASMFRVLIGTYYDNFCAIARLIGTKTCRQVYEFRVKESSIIAPVPTED | |
| | VDTPPRKKKRKHRLWAAHCRKIQLKKDGSSNHVYNYQPCDHPRQPC | |
| | DSSCPCVIAQNFCEKFCQCSSECQNRFPGCRCKAQCNTKQCPCYLAVR | |
| | ECDPDLCLTCGAADHWDSKNVSCKNCSIQRGSKKHLLLAPSDVAGWG | |
| | IFIKDPVQKNEFISEYCGEIISQDEADRRGKVYDKYMCSFLFNLNNDFV | |
| | VDATRKGNKIRFANHSVNPNCYAKVMMVNGDHRIGIFAKRAIQTGEE | |
| | LFFDYRYSQADALKYVGIEREMEIP | |
| 2 | TEDVDTPPRKKKRKHRLWAAHCRKIQLKKDGSSNHVYNYQPCDHPR | Ezh2[SET] (amino |
| | QPCDSSCPCVIAQNFCEKFCQCSSECQNRFPGCRCKAQCNTKQCPCYL | acids 482-746 of |
| | AVRECDPDLCLTCGAADHWDSKNVSCKNCSIQRGSKKHLLLAPSDVA | NP_031997.2) |
| | GWGIFIKDPVQKNEFISEYCGEIISQDEADRRGKVYDKYMCSFLFNLNN | |
| | DFVVDATRKGNKIRFANHSVNPNCYAKVMMVNGDHRIGIFAKRAIQT | |
| | GEELFFDYRYSQADALKYVGIEREMEIP | |
| 3 | MSRRKQSNPRQIKRSLGDMEAREEVQLVGASHMEQKATAPEAPSP | FOG1[1-45] (amino |
| | | acids 1-45 of |
| | | AAN45858.1) |
| 4 | PRQNLKCVRILKQFHKDLERELLRRHHRSKTPRHLDPSLANYLVQKAK | SUV[SET] (amino |
| | QRRALRRWEQELNAKRSHLGRITVENEVDLDGPPRAFVYINEYRVGE | acids 76-412 of |
| | GITLNQVAVGCECQDCLWAPTGGCCPGASLHKFAYNDQGQVRLRAG | NP_003164.1) |
| | LPIYECNSRCRCGYDCPNRVVQKGIRYDLCIFRTDDGRGWGVRTLEKI | |
| | RKNSFVMEYVGEIITSEEAERRGQIYDRQGATYLFDLDYVEDVYTVDA | |
| | AYYGNISHFVNHSCDPNLQVYNVFIDNLDERLPRIAFFATRTIRAGEEL | |
| | TFDYNMQVDPVDMESTRMDSNFGLAGLPGSPKKRVRIECKCGTESCR | |
| | KYLF | |
| 5 | GSAAIAEVLLNARCDLHAVNYHGDTPLHIAARESYHDCVLLFLSRGAN | G9A[SET] (amino acids |
| | PELRNKEGDTAWDLTPERSDVWFALQLNRKLRLGVGNRAIRTEKIICR | 829-1209 of |
| | DVARGYENVPIPCVNGVDGEPCPEDYKYISENCETSTMNIDRNITHLQH | NP_006700.3) |
| | CTCVDDCSSSNCLCGQLSIRCWYDKDGRLLQEFNKIEPPLIFECNQACS | |
| | CWRNCKNRVVQSGIKVRLQLYRTAKMGWGVRALQTIPQGTFICEYVG | |
| | ELISDAEADVREDDSYLFDLDNKDGEVYCIDARYYGNISRFINHLCDPN | |
| | IIPVRVFMLHQDLRFPRIAFFSSRDIRTGEELGFDYGDRFWDIKSKYFTC | |
| | QCGSEKCKHSAEAIALEQSRLARLDPHPELLPELGSLPPVN | |
| 6 | RAPSRLQMFFANNHDQEFDPPKVYPPVPAEKRKPIRVLSLFDGIATGLL | DNMT3A (amino acids |
| | VLKDLGIQVDRYIASEVCEDSITVGMVRHQGKIMYVGDVRSVTQKHIQ | 602-912 of |
| | EWGPFDLVIGGSPCNDLSIVNPARKGLYEGTGRLFFEFYRLLHDARPKE | NP_072046.2) |
| | GDDRPFFWLFENVVAMGVSDKRDISRFLESNPVMIDAKEVSAAHRAR | |
| | YFWGNLPGMNRPLASTVNDKLELQECLEHGRIAKFSKVRTITTRSNSIK | |
| | QGKDQHFPVFMNEKEDILWCTEMERVFGFPVHYTDVSNMSRLARQRL | |
| | LGRSWSVPVIRHLFAPLKEYFACV | |
| 7 | TLVTFKDVFVDFTREEWKLLDTAQQIVYRNVMLENYKNLVSLGYQLT | KRAB (amino acids 12- |
| | KPDVILRLEKGEEPWLVEREIHQETHP | 85 of NP_056209.2) |
| 8 | MPKKKRKVGGSGGSDYKDHDGDYKDHDIDYKDDDDKGGGSGGGSG | Improved dCas9 (amino |
| | TGGSGGSGGSGGSGGSGRPMDKKYSIGLAIGTNSVGWAVITDEYKV | acids 2-9, SV40 NLS; |
| | PSKKFKVLGNTDRHSIKKNLIGALLFDSGETAEATRLKRTARRRYT | amino acids 15-36, |
| | RRKNRICYLQEIFSNEMAKVDDSFFHRLEESFLVEEDKKHERHPIF | 3xFLAG; amino acids |
| | GNIVDEVAYHEKYPTIYHLRKKLVDSTDKADLRLIYLALAHMIKFR | 65-1432 (bold), dCas9 |
| | GHFLIEGDLNPDNSDVDKLFIQLVQTYNQLFEENPINASGVDAKAIL | (D10A, H840A); amino |
| | SARLSKSRRLENLIAQLPGEKKNGLFGNLIALSLGLTPNFKSNFDLA | acids 1458-1473, |
| | EDAKLQLSKDTYDDDLDNLLAQIGDQYADLFLAAKNLSDAILLSDI | Neoplasmin NLS) |
| | LRVNTEITKAPLSASMIKRYDEHHQDLTLLKALVRQQKKKRKVGL | |
| | PEKYKEIFFDQSKNGYAGYIDGGASQEEFYKFIKPILEKMDGTEEL | |
| | LVKLNREDLLRKQRTFDNGSIPHQIHLGELHAILRRQEDFYPFLKD | |
| | NREKIEKILTFRIPYWGPLARGNSRFAWMTRKSEETITPWNFEEV | |
| | VDKGASAQSFIERMTNFDKNLPNEKVLPKHSLLYEYFTVYNELTK | |
| | VKYVTEGMRKPAFLSGEQKKAIVDLLFKTNRKVTVKQLKEDYFK | |
| | KIECFDSVEISGVEDRFNASLGTYHDLLKIIKDKDFLDNEENEDILE | |
| | DIVLTLTLFEDREMIEERLKTYAHLFDDKVMKQLKRRRYTGWGR | |
| | LSRKLINGIRDKQSGKTILDFLKSDGFANRNFMQLIHDDSLTFKEDI | |
| | QKAQVSGQGDSLHEHIANLAGSPAIKKGILQTVKVVDELVKVMGR | |
| | HKPENIVIEMARENQTTQKGQKNSRERMKRIEEGIKELGSQILKEH | |
| | PVENTQLQNEKLYLYYLQNGRDMYVDQELDINRLSDYDVDAIVPQ | |
| | SFLKDDSIDNKVLTRSDKNRGKSDNVPSEEVVKKMKNYWRQLLNA | |
| | KLITQRKFDNLTKAERGGLSELDKAGFIKRQLVETRQITKHVAQIL | |
| | DSRMNTKYDENDKLIREVKVITLKSKLVSDFRKDFQFYKVREINNY | |
| | HHAHDAYLNAVVGTALIKKYPKLESEFVYGDYKVYDVRKMIAKSE | |
| | QEIGKATAKYFFYSNIMNFFKTEITLANGEIRKRPLIETNGETGEIV | |
| | WDKGRDFATVRKVLSMPQVNIVKKTEVQTGGFSKESILPKRNSDK | |
| | LIARKKDWDPKKYGGFDSPTVAYSVLVVAKVEKGKSKKLKSVKE | |
| | LLGITIMERSSFEKNPIDFLEAKGYKEVKKDLIIKLPKYSLFELENG | |
| | RKRMLASAGELQKGNELALPSKYVNFLYLASHYEKLKGSPEDNEQ | |
| | KQLFVEQHKHYLDEIIEQISEFSKRVILADANLDKVLSAYNKHRDK | |
| | PIREQAENIIHLFTLTNLGAPAAFKYFDTTIDRKRYTSTKEVLDATL | |
| | IHQSITGLYETRIDLSQLGGDGGSGGSGGSGGSGGSASGGGSGGGSK | |
| | RPAATKKAGQAKKKKGGSGSGATNFSLLKQAGDVEENPGPAAA | |
| 9 | MPKKKRKVGGSGGSDYKDHDGDYKDHDIDYKDDDDKGGGSGGGSG | ED-dCas9: location |
| | TZSTGGSGGSGGSGGSGGSGRPMDKKYSIGLAIGTNSVGWAVITDEY | effector domain |
| | KVPSKKFKVLGNTDRHSIKKNLIGALLFDSGETAEATRLKRTARRR | (denoted by bold |
| | YTRRKNRICYLQEIFSNEMAKVDDSFFHRLEESFLVEEDKKHERHP | underlined Z) fused to |
| | IFGNIVDEVAYHEKYPTIYHLRKKLVDSTDKADLRLIYLALAHMIK | N-terminus of dCas9. |
| | FRGHFLIEGDLNPDNSDVDKLFIQLVQTYNQLFEENPINASGVDAK | Underlined denotes |
| | AILSARLSKSRRLENLIAQLPGEKKNGLFGNLIALSLGLTPNFKSNF | (GGS)₅ amino acid |
| | DLAEDAKLQLSKDTYDDDLDNLLAQIGDQYADLFLAAKNLSDAIL | linkers (SEQ ID NO: |
| | LSDILRVNTEITKAPLSASMIKRYDEHHQDLTLLKALVRQQKKKR | 75). Bold denotes |
| | KVGLPEKYKEIFFDQSKNGYAGYIDGGASQEEFYKFIKPILEKMDG | dCas9. Amino acids 2- |
| | TEELLVKLNREDLLRKQRTFDNGSIPHQIHLGELHAILRRQEDFYP | 9: SV40 NLS. Amino |
| | FLKDNREKIEKILTFRIPYYVGPLARGNSRFAWMTRKSEETITPWN | acids 15-36: 3xFLAG. |
| | FEEVVDKGASAQSFIERMTNFDKNLPNEKVLPKHSLLYEYFTVYNE | Italics denote |
| | LTKVKYVTEGMRKPAFLSGEQKKAIVDLLFKTNRKVTVKQLKED | Neoplasmin NLS. |
| | YFKKIECFDSVEISGVEDRFNASLGTYHDLLKIIKDKDFLDNEENED | |
| | ILEDIVLTLTLFEDREMIEERLKTYAHLFDDKVMKQLKRRRYTGW | |
| | GRLSRKLINGIRDKQSGKTILDFLKSDGFANRNFMQLIHDDSLTFK | |
| | EDIQKAQVSGQGDSLHEHIANLAGSPAIKKGILQTVKVVDELVKV | |
| | MGRHKPENIVIEMARENQTTQKGQKNSRERMKRIEEGIKELGSQI | |
| | LKEHPVENTQLQNEKLYLYYLQNGRDMYVDQELDINRLSDYDVD | |
| | AIVPQSFLKDDSIDNKVLTRSDKNRGKSDNVPSEEVVKKMKNYWR | |
| | QLLNAKLITQRKFDNLTKAERGGLSELDKAGFIKRQLVETRQITK | |
| | HVAQILDSRMNTKYDENDKLIREVKVITLKSKLVSDFRKDFQFYKV | |
| | REINNYHHAHDAYLNAVVGTALIKKYPKLESEFVYGDYKVYDVRK | |
| | MIAKSEQEIGKATAKYFFYSNIMNFFKTEITLANGEIRKRPLIETNG | |
| | ETGEIVWDKGRDFATVRKVLSMPQVNIVKKTEVQTGGFSKESILP | |
| | KRNSDKLIARKKDWDPKKYGGFDSPTVAYSVLVVAKVEKGKSKK | |
| | LKSVKELLGITIMERSSFEKNPIDFLEAKGYKEVKKDLIIKLPKYSL | |
| | FELENGRKRMLASAGELQKGNELALPSKYVNFLYLASHYEKLKGS | |
| | PEDNEQKQLFVEQHKHYLDEIIEQISEFSKRVILADANLDKVLSAYN | |
| | KHRDKPIREQAENIIHLFTLTNLGAPAAFKYFDTTIDRKRYTSTKEV | |
| | LDATLIHQSITGLYETRIDLSQLGGDGGSGGSGGSGGSGGSASGGGS | |
| | GGGSKRPAATKKAGQAKKKKGGSGSGATNFSLLKQAGDVEENPGPAAA | |
| 10 | MPKKKRKVGGSGGSDYKDHDGDYKDHDIDYKDDDDKGGGSGGGSG | dCas9-ED: location |
| | TGGSGGSGGSGGSGGSGRPMDKKYSIGLAIGTNSVGWAVITDEYKV | effector domain |
| | PSKKFKVLGNTDRHSIKKNLIGALLFDSGETAEATRLKRTARRRYT | (denoted by bold |
| | RRKNRICYLQEIFSNEMAKVDDSFFHRLEESFLVEEDKKHERHPIF | underlined Z) fused to |
| | GNIVDEVAYHEKYPTIYHLRKKLVDSTDKADLRLIYLALAHMIKFR | C-terminus of dCas9. |
| | GHFLIEGDLNPDNSDVDKLFIQLVQTYNQLFEENPINASGVDAKAIL | Underlined denotes |
| | SARLSKSRRLENLIAQLPGEKKNGLFGNLIALSLGLTPNFKSNFDLA | (GGS)₅ amino acid |
| | EDAKLQLSKDTYDDDLDNLLAQIGDQYADLFLAAKNLSDAILLSDI | linkers (SEQ ID NO: |
| | LRVNTEITKAPLSASMIKRYDEHHQDLTLLKALVRQQKKKRKVGL | 75). Bold denotes |
| | PEKYKEIFFDQSKNGYAGYIDGGASQEEFYKFIKPILEKMDGTEEL | dCas9. Amino acids 2- |
| | LVKLNREDLLRKQRTFDNGSIPHQIHLGELHAILRRQEDFYPFLKD | 9: SV40 NLS. Amino |
| | NREKIEKILTFRIPYYVGPLARGNSRFAWMTRKSEETITPWNFEEV | acids 15-36: 3xFLAG. |
| | VDKGASAQSFIERMTNFDKNLPNEKVLPKHSLLYEYFTVYNELTK | Italics denote |
| | VKYVTEGMRKPAFLSGEQKKAIVDLLFKTNRKVTVKQLKEDYFK | Neoplasmin NLS. |
| | KIECFDSVEISGVEDRFNASLGTYHDLLKIIKDKDFLDNEENEDILE | |
| | DIVLTLTLFEDREMIEERLKTYAHLFDDKVMKQLKRRRYTGWGR | |
| | LSRKLINGIRDKQSGKTILDFLKSDGFANRNFMQLIHDDSLTFKEDI | |
| | QKAQVSGQGDSLHEHIANLAGSPAIKKGILQTVKVVDELVKVMGR | |
| | HKPENIVIEMARENQTTQKGQKNSRERMKRIEEGIKELGSQILKEH | |
| | PVENTQLQNEKLYLYYLQNGRDMYVDQELDINRLSDYDVDAIVPQ | |
| | SFLKDDSIDNKVLTRSDKNRGKSDNVPSEEVVKKMKNYWRQLLNA | |
| | KLITQRKFDNLTKAERGGLSELDKAGFIKRQLVETRQITKHVAQIL | |
| | DSRMNTKYDENDKLIREVKVITLKSKLVSDFRKDFQFYKVREINNY | |
| | HHAHDAYLNAVVGTALIKKYPKLESEFVYGDYKVYDVRKMIAKSE | |
| | QEIGKATAKYFFYSNIMNFFKTEITLANGEIRKRPLIETNGETGEIV | |
| | WDKGRDFATVRKVLSMPQVNIVKKTEVQTGGFSKESILPKRNSDK | |
| | LIARKKDWDPKKYGGFDSPTVAYSVLVVAKVEKGKSKKLKSVKE | |
| | LLGITIMERSSFEKNPIDFLEAKGYKEVKKDLIIKLPKYSLFELENG | |
| | RKRMLASAGELQKGNELALPSKINNFLYLASHYEKLKGSPEDNEQ | |
| | KQLFVEQHKHYLDEIIEQISEFSKRVILADANLDKVLSAYNKHRDK | |
| | PIREQAENIIHLFTLTNLGAPAAFKYFDTTIDRKRYTSTKEVLDATL | |
| | IHQSITGLYETRIDLSQLGGDGGSGGSGGSGGSGGSASZTSGGGSGG | |
| | GSKRPAATKKAGQAKKKKGGSGSGATNFSLLKQAGDVEENPGPAAA | |
| 11 | GGTGGCGGGTCCGGCGGTGGATCCGGTACCGGCAGCGCCGCCATCG | G9A[SET] (KpnI) |
| | CCGAAGTCCTTCTG | forward oligo for |
| | | Gibson cloning of ED- |
| | | dCas9 |
| 12 | GGTGGCGGGTCCGGCGGTGGATCCGGTACCCCACGGCAGAATCTCA | SUV[SET] (KpnI) |
| | AGTGTGTGCGTATC | forward oligo for |
| | | Gibson cloning of ED- |
| | | dCas9 |
| 13 | GGTGGCGGGTCCGGCGGTGGATCCGGTACCATGACTGAGGATGTAG | Ezh2[SET] (KpnI) |
| | ACACTCCT | forward oligo for |
| | | Gibson cloning of ED- |
| | | dCas9 |
| 14 | GGTGGCGGGTCCGGCGGTGGATCCGGTACCACACTGGTGACCTTCA | KRAB (KpnI) forward |
| | AGGATGTATTTGTG | oligo for Gibson |
| | | cloning of ED-dCas9 |
| 15 | GGTGGCGGGTCCGGCGGTGGATCCGGTACCCGCGCCCCCTCCCGGC | DNMT3A (KpnI) |
| | TCCAGATG | forward oligo for |
| | | Gibson cloning of ED- |
| | | dCas9 |
| 16 | GGTGGCGGGTCCGGCGGTGGATCCGGTACCATGTCCAGGCGGAAA | FOG1 (KpnI) forward |
| | CAGAGC | oligo for Gibson |
| | | cloning of ED-dCas9 |
| 17 | ACCGCCGCTTCCACCACTCCCTCCGGTACTTGTGTTGACAGGGGGC | G9A[SET] (KpnI) |
| | AGGGAGCCGAGCTC | reverse oligo for Gibson |
| | | cloning of ED-dCas9 |
| 18 | ACCGCCGCTTCCACCACTCCCTCCGGTACTGAAGAGGTATTTGCGG | SUV[SET] (KpnI) |
| | CAGGACTCAGTCC | reverse oligo for Gibson |
| | | cloning of ED-dCas9 |
| 19 | ACCGCCGCTTCCACCACTCCCTCCGGTACTAGGGATTTCCATTTCTC | Ezh2[SET] (KpnI) |
| | GTTC | reverse oligo for Gibson |
| | | cloning of ED-dCas9 |
| 20 | ACCGCCGCTTCCACCACTCCCTCCGGTACTAGGATGGGTCTCTTGGT | KRAB (KpnI) reverse |
| | GAATTTCTCTCTC | oligo for Gibson |
| | | cloning of ED-dCas9 |
| 21 | ACCGCCGCTTCCACCACTCCCTCCGGTACTCACACACGCAAAATAC | DNMT3A (KpnI) |
| | TCCTTCAG | reverse oligo for Gibson |
| | | cloning of ED-dCas9 |
| 22 | ACCGCCGCTTCCACCACTCCCTCCGGTACTAGGGCTCGGGGCTTCA | FOG1 (KpnI) reverse |
| | GGTG | oligo for Gibson |
| | | cloning of ED-dCas9 |
| 23 | /5Phos/GAATTCATCTCAGAAGCCTGTGGGGAGATTATTTCTCAG | Ezh2[SET-Y641A] |
| | | forward oligo for |
| | | mutagenesis of catalytic |
| | | residues in Ezh2-SET |
| | | domain (mutation site |
| | | bold and underlined) |
| 24 | /5Phos/GGTGAAGAGTTGTTTTTTGATTTCAGATACAGCCAGGCTGAT | Ezh2[SET-Y726F] |
| | GC | forward oligo for |
| | | mutagenesis of catalytic |
| | | residues in Ezh2-SET |
| | | domain (mutation site |
| | | bold and underlined) |
| 25 | /5Phos/CTGAGAAATAATCTCCCCACAGGCTTCTGAGATGAATTC | Ezh2[SET-Y641A] |
| | | reverse oligo for |
| | | mutagenesis of catalytic |
| | | residues in Ezh2-SET |
| | | domain (mutation site |
| | | bold and underlined) |
| 26 | /5Phos/GCATCAGCCTGGCTGTATCTGAAATCAAAAAACAACTCTTCA | Ezh2[SET-Y726F] |
| | CC | reverse oligo for |
| | | mutagenesis of catalytic |
| | | residues in Ezh2-SET |
| | | domain (mutation site |
| | | bold and underlined) |
| 27 | GGATCCGGGGGGAGCGGAGGGAGCGCTAGCGGCAGCGCCGCCATC | G9A[SET] (NheI) |
| | GCCGAAGTCCTTCTG | forward oligo for |
| | | Gibson cloning of |
| | | dCas9-ED |
| 28 | GGATCCGGGGGGAGCGGAGGGAGCGCTAGCCCACGGCAGAATCTC | SUV[SET] (NheI) |
| | AAGTGTGTGCGTATC | forward oligo for |
| | | Gibson cloning of |
| | | dCas9-ED |
| 29 | GGATCCGGGGGGAGCGGAGGGAGCGCTAGCATGTCCAGGCGGAAA | FOG1 (NheI) forward |
| | CAGAGC | oligo for Gibson |
| | | cloning of dCas9-ED |
| 30 | GGATCCGGGGGGAGCGGAGGGAGCGCTAGCCGCGCCCCCTCCCGG | DNMT3A (NheI) |
| | CTCCAGATG | forward oligo for |
| | | Gibson cloning of |
| | | dCas9-ED |
| 31 | TTAGATCCACCTCCGGAGCCTCCACCGGATGTGTTGACAGGGGGCA | G9A[SET] (NheI) |
| | GGGAGCCGAGCTC | reverse oligo for Gibson |
| | | cloning of dCas9-ED |
| 32 | TTAGATCCACCTCCGGAGCCTCCACCGGAGAAGAGGTATTTGCGGC | SUV[SET] (NheI) |
| | AGGACTCAGTCCC | reverse oligo for Gibson |
| | | cloning of dCas9-ED |
| 33 | TTAGATCCACCTCCGGAGCCTCCACCGGAAGGGCTCGGGGCTTCAG | FOG1 (NheI) reverse |
| | GTG | oligo for Gibson |
| | | cloning of dCas9-ED |
| 34 | TTAGATCCACCTCCGGAGCCTCCACCGGACACACACGCAAAATACT | DNMT3A (NheI) |
| | CCTTCAG | reverse oligo for Gibson |
| | | cloning of dCas9-ED |
| 35 | GCTAGGTCTCTCTATCGGTACCATGTCCAGGCGGAAACAGAG | 2xFOG1-1, 3xFOG1-1, |
| | | 4xFOG1-1 forward |
| | | Gibson cloning oligo |
| 36 | GATGGTCTCGGGTCGATGTCCAGGCGGAAACAGAG | 2xFOG1-2, 3xFOG1-2, |
| | | 4xFOG1-2 forward |
| | | Gibson cloning oligo |
| 37 | GATGGTCTCGGCTCCATGTCCAGGCGGAAACAGAG | 3xFOG1-3, 4xFOG1-3 |
| | | forward Gibson cloning |
| | | oligo |
| 38 | GATGGTCTCGGAAGCATGTCCAGGCGGAAACAGAG | 4xFOG1-4 forward |
| | | Gibson cloning oligo |
| 39 | GATGGTCTCCGACCCAGGGCTCGGGGCTTCAGGTG | 2xFOG1-1, 3xFOG1-1, |
| | | 4xFOG1-1 reverse |
| | | Gibson cloning oligo |
| 40 | CATGGTCTCACGCCAGGCCGGCCGCTGCCGCCTGAGCCACCAGAAC | 2xFOG1-2, 3xFOG1-3, |
| | CGCCGCTTCCACCACTCCCTCCAGGGCTCGGGGCTTCAGGTG | 4xFOG1-2, 4xFOG1-4 |
| | | reverse Gibson cloning |
| | | oligo |
| 41 | GATGGTCTCGGAGCCAGGGCTCGGGGCTTCAGGTG | 3xFOG1-2 reverse |
| | | Gibson cloning oligo |
| 42 | GATGGTCTCGCTTCCAGGGCTCGGGGCTTCAGGTG | 4xFOG1-3 reverse |
| | | Gibson cloning oligo |
| 43 | GAATTTATCCCGGACTCCGGGG | HER2 gRNA1 target |
| | | site (including PAM) |
| | | (gRNA design G-N19) |
| 44 | GTTGGAATGCAGTTGGAGGGGG | HER2 gRNA2 target |
| | | site (including PAM) |
| | | (gRNA design G-N19) |
| 45 | ATTCCAGAAGATATGCCCCGGG | HER2 gRNA3 target |
| | | site (including PAM) |
| | | (gRNA design G-N19) |
| 46 | TTTAAGATAAAACCTGAGACTTAAAAG | HER2 crRNA 1 Cpf1 |
| | | target site (including |
| | | PAM) |
| 47 | TTTCTCCCTCTCTTCGCGCAGGCCTGG | HER2 crRNA 2 Cpf1 |
| | | target site (including |
| | | PAM) |
| 48 | TTTCTCCGGTCCCAATGGAGGGGAATC | HER2 crRNA 3 Cpf1 |
| | | target site (including |
| | | PAM) |
| 49 | GGTGGCTCAGGCGGCAGCGGCCGGCCAATGACACAGTTCGAGGGC | Forward primer to |
| | TT | generate dCPf1 |
| | | (D908A) (left) |
| 50 | TCGGCATCGCCCGGGGCGAGAGAAACCTGA | Forward primer to |
| | | generate dCPf1 |
| | | (D908A) (right) |
| 51 | CTCGCCCCGGGCGATGCCGATGATAGGTGTC | Reverse primer to |
| | | generate dCPf1 |
| | | (D908A) (left) |
| 52 | CACCTCCGGAGCCTCCACCGCTAGCGCTCCCTCCGCTCCCCCCGGAT | Reverse primer to |
| | CCTCCTGAACCTCCACTACCACCGTTGCGCAGCTCCTGGATG | generate dCPf1 |
| | | (D908A) (right) |
| 53 | GGGAAACCTGGAACTCACCT | HER2 forward primer |
| 54 | GACCTGCCTCACTTGGTTGT | HER2 reverse primer |
| 55 | CTGGCCGTAAACTGCTTTGT | EPCAM forward primer |
| 56 | TCCCAAGTTTTGAGCCATTC | EPCAM reverse primer |
| 57 | AAACACAAACTTGAACAGCTAC | MYC forward primer |
| 58 | ATTTGAGGCAGTTTACATTATGG | MYC reverse primer |
| 59 | AATCCCATCACCATCTTCCA | GAPDH forward primer |
| 60 | CTCCATGGTGGTGAAGACG | GAPDH reverse primer |
| 61 | TTGGAATGCAGTTGGAGGGG | HER2-ChIP forward |
| | | primer |
| 62 | GGTTTCTCCGGTCCCAATGG | HER2-ChIP reverse |
| | | primer |
| 63 | GGAGGGGGTAGAGTTATTAGTTTTT | HER2-BSP forward |
| | | primer |
| 64 | AAATAACAACTCCCAACTTCACTTT | HER2-BSP reverse |
| | | primer |
| 65 | DYKDDDDK | FLAG motif |
| 66 | DYKDHDGDYKDHDIDYKDDDDK | 3xFLAG peptide |
| 67 | MGQTGKKSEKGPVCWRKRVKSEYMRLRQLKRFRRADEVKSMFSSNR | Ezh2 amino acid |
| | QKILERTEILNQEWKQRRIQPVHILTSVSSLRGTRECSVTSDLDFPTQVIP | sequence AAH10858.1 |
| | LKTLNAVASVPIMYSWSPLQQNFMVEDETVLHNIPYMGDEVLDQDGT | |
| | FIEELIKNYDGKVHGDRECGFINDEIFVELVNALGQYNDDDDDDDGDD | |
| | PEEREEKQKDLEDHRDDKESRPPRKFPSDKIFEAISSMFPDKGTAEELK | |
| | EKYKELTEQQLPGALPPECTPNIDGPNAKSVQREQSLHSFHTLFCRRCF | |
| | KYDCFLHRKCNYSFHATPNTYKRKNTETALDNKPCGPQCYQHLEGAK | |
| | EFAAALTAERIKTPPKRPGGRRRGRLPNNSSRPSTPTINVLESKDTDSDR | |
| | EAGTETGGENNDKEEEEKKDETSSSSEANSRCQTPIKMKPNIEPPENVE | |
| | WSGAEASMFRVLIGTYYDNFCAIARLIGTKTCRQVYEFRVKESSIIAPA | |
| | PAEDVDTPPRKKKRKHRLWAAHCRKIQLKKDGSSNHVYNYQPCDHP | |
| | RQPCDSSCPCVIAQNFCEKFCQCSSECQNRFPGCRCKAQCNTKQCPCY | |
| | LAVRECDPDLCLTCGAADHWDSKNVSCKNCSIQRGSKKHLLLAPSDV | |
| | AGWGIFIKDPVQKNEFISEYCGEIISQDEADRRGKVYDKYMCSFLFNLN | |
| | NDFVVDATRKGNKIRFANHSVNPNCYAKVMMVNGDHRIGIFAKRAIQ | |
| | TGEELFFDYRYSQADALKYVGIEREMEIP | |
| 68 | PKKKRKV | Monopartite NLS |
| | | sequence |
| 69 | PKKKRKVG | Monopartite NLS |
| | | sequence |
| 70 | KRPAATKKAGQAKKKK | Bipartite NLS sequence |
| 71 | GGS | Amino acid linker |
| | | sequence ((GGS)₁) |
| 72 | GGSGGS | Amino acid linker |
| | | sequence ((GGS)₂) |
| 73 | GGSGGSGGS | Amino acid linker |
| | | sequence ((GGS)₃) |
| 74 | GGSGGSGGSGGS | Amino acid linker |
| | | sequence ((GGS)₄) |
| 75 | GGSGGSGGSGGSGGS | Amino acid linker |
| | | sequence ((GGS)₅) |
| 76 | GGSGGSGGSGGSGGSGGS | Amino acid linker |
| | | sequence ((GGS)₆) |
| 77 | GGSGGSGGSGGSGGSGGSGGS | Amino acid linker |
| | | sequence ((GGS)₇) |
| 78 | GGSGGSGGSGGSGGSGGSGGSGGS | Amino acid linker |
| | | sequence ((GGS)₈) |
| 79 | GGSGGSGGSGGSGGSGGSGGSGGSGGS | Amino acid linker |
| | | sequence ((GGS)₉) |
| 80 | GGSGGSGGSGGSGGSGGSGGSGGSGGSGGS | Amino acid linker |
| | | sequence ((GGS)₁₀) |
| 81 | MPKKKRKVGGSGGSDYKDHDGDYKDHDIDYKDDDDKGGGSGGGSG | Ezh2[FL]-dCas9: |
| | TMGQTGKKSEKGPVCWRKRVKSEYMRLRQLKRFRRADEVKTMF | location effector |
| | SSNRQKILERTETLNQEWKQRRIQPVHIMTSVSSLRGTRECSVTSD | domain (denoted by |
| | LDFPAQVIPLKTLNAVASVPIMYSWSPLQQNFMVEDETVLHNIPYM | bold underlined |
| | GDEVLDQDGTFIEELIKNYDGKVHGDRECGFINDEIFVELVNALGQ | sequence) fused to N- |
| | YNDDDDDDDGDDPDEREEKQKDLEDNRDDKETCPPRKFPADKIFE | terminus of dCas9. |
| | AISSMFPDKGTAEELKEKYKELTEQQLPGALPPECTPNIDGPNAKS | Underlined denotes |
| | VQREQSLHSFHTLFCRRCFKYDCFLHPFHATPNTYKRKNTETALD | (GGS)₅ amino acid |
| | NKPCGPQCYQHLEGAKEFAAALTAERIKTPPKRPGGRRRGRLPNN | linkers (SEQ ID NO: |
| | SSRPSTPTISVLESKDTDSDREAGTETGGENNDKEEEEKKDETSSSS | 75). Bold denotes |
| | EANSRCQTPIKMKPNIEPPENVEWSGAEASMFRVLIGTYYDNFCAI | dCas9. Amino acids 2- |
| | ARLIGTKTCRQVYEFRYKESSIIAPYPTEDVDTPPRKKKRKHRLWA | 9: SV40 NLS. Amino |
| | AHCRKIQLKKDGSSNHVYNYQPCDHPRQPCDSSCPCVIAQNFCEK | acids 15-36: 3xFLAG. |
| | FCQCSSECQNRFPGCRCKAQCNTKQCPCYLAVRECDPDLCLTCGA | Italics denote |
| | ADHWDSKNVSCKNCSIQRGSKKHLLLAPSDVAGWGIFIKDPVQKN | Neoplasmin NLS. |
| | EFISEYCGEIISQDEADRRGKVYDKYMCSFLFNLNNDFVVDATRKG | |
| | NKIRFANHSVNPNCYAKVMMVNGDHRIGIFAKRAIQTGEELFFDY | |
| | RYSQADALKYVGIEREMEIPSTGGSGGSGGSGGSGGSGRPMDKKYSI | |
| | GLAIGTNSVGWAVITDEYKVPSKKFKVLGNTDRHSIKKNLIGALLF | |
| | DSGETAEATRLKRTARRRYTRRKNRICYLQEIFSNEMAKVDDSFFH | |
| | RLEESFLVEEDKKHERHPIFGNIVDEVAYHEKYPTIYHLRKKLVDS | |
| | TDKADLRLIYLALAHMIKFRGHFLIEGDLNPDNSDVDKLFIQLVQT | |
| | YNQLFEENPINASGVDAKAILSARLSKSRRLENLIAQLPGEKKNGLF | |
| | GNLIALSLGLTPNFKSNFDLAEDAKLQLSKDTYDDDLDNLLAQIGD | |
| | QYADLFLAAKNLSDAILLSDILRVNTEITKAPLSASMIKRYDEHHQD | |
| | LTLLKALVRQQKKKRKVGLPEKYKEIFFDQSKNGYAGYIDGGASQ | |
| | EEFYKFIKPILEKMDGTEELLVKLNREDLLRKQRTFDNGSIPHQIH | |
| | LGELHAILRRQEDFYPFLKDNREKIEKILTFRIPYINGPLARGNSRF | |
| | AWMTRKSEETITPWNFEEVVDKGASAQSFIERMTNFDKNLPNEKV | |
| | LPKHSLLYEYFTVYNELTKVKYVTEGMRKPAFLSGEQKKAIVDLL | |
| | FKTNRKVTVKQLKEDYFKKIECFDSVEISGVEDRFNASLGTYHDLL | |
| | KIIKDKDFLDNEENEDILEDIVLTLTLFEDREMIEERLKTYAHLFDD | |
| | KVMKQLKRRRYTGWGRLSRKLINGIRDKQSGKTILDFLKSDGFAN | |
| | RNFMQLIHDDSLTFKEDIQKAQVSGQGDSLHEHIANLAGSPAIKKG | |
| | ILQTVKVVDELVKVMGRHKPENIVIEMARENQTTQKGQKNSRER | |
| | MKRIEEGIKELGSQILKEHPVENTQLQNEKLYLYYLQNGRDMYVD | |
| | QELDINRLSDYDVDAIVPQSFLKDDSIDNKVLTRSDKNRGKSDNVPS | |
| | EEVVKKMKNYWRQLLNAKLITQRKFDNLTKAERGGLSELDKAGF | |
| | IKRQLVETRQITKHVAQILDSRMNTKYDENDKLIREVKVITLKSKL | |
| | VSDFRKDFQFYKVREINNYHHAHDAYLNAVVGTALIKKYPKLESE | |
| | FVYGDYKVYDVRKMIAKSEQEIGKATAKYFFYSNIMNFFKTEITLA | |
| | NGEIRKRPLIETNGETGEIVWDKGRDFATVRKVLSMPQVNIVKKT | |
| | EVQTGGFSKESILPKRNSDKLIARKKDWDPKKYGGFDSPTVAYSVL | |
| | VVAKVEKGKSKKLKSVKELLGITIMERSSFEKNPIDFLEAKGYKEV | |
| | KKDLIIKLPKYSLFELENGRKRMLASAGELQKGNELALPSKYVNFL | |
| | YLASHYEKLKGSPEDNEQKQLFVEQHKHYLDEIIEQISEFSKRVILA | |
| | DANLDKVLSAYNKHRDKPIREQAENIIHLFTLTNLGAPAAFKYFDT | |
| | TIDRKRYTSTKEVLDATLIHQSITGLYETRIDLSQLGGDGGSGGSGG | |
| | SGGSGGSASGGGSGGGSKRPAATKKAGQAKKKKGGSGSGATNFSLLKQ | |
| | AGDVEENPGPAAA | |
| 82 | MPKKKRKVGGSGGSDYKDHDGDYKDHDIDYKDDDDKGGGSGGGSG | Ezh2[SET]-dCas9: |
| | TTEDVDTPPRKKKRKHRLWAAHCRKIQLKKDGSSNHVYNYQPCD | location effector |
| | HPRQPCDSSCPCVIAQNFCEKFCQCSSECQNRFPGCRCKAQCNTK | domain (denoted by |
| | QCPCYLAVRECDPDLCLTCGAADHWDSKNVSCKNCSIQRGSKKH | bold underlined |
| | LLLAPSDVAGWGIFIKDPVQKNEFISEYCGEIISQDEADRRGKVYD | sequence) fused to N- |
| | KYMCSFLFNLNNDFVVDATRKGNKIRFANHSVNPNCYAKVMMVN | terminus of dCas9. |
| | GDHRIGIFAKRAIQTGEELFFDYRYSQADALKINGIEREMEIPSTGG | Underlined denotes |
| | SGGSGGSGGSGGSGRPMDKKYSIGLAIGTNSVGWAVITDEYKVPSK | (GGS)₅ amino acid |
| | KFKVLGNTDRHSIKKNLIGALLFDSGETAEATRLKRTARRRYTRR | linkers (SEQ ID NO: |
| | KNRICYLQEIFSNEMAKVDDSFFHRLEESFLVEEDKKHERHPIFGNI | 75). Bold denotes |
| | VDEVAYHEKYPTIYHLRKKLVDSTDKADLRLIYLALAHMIKFRGH | dCas9. Amino acids 2- |
| | FLIEGDLNPDNSDVDKLFIQLVQTYNQLFEENPINASGVDAKAILSA | 9: SV40 NLS. Amino |
| | RLSKSRRLENLIAQLPGEKKNGLFGNLIALSLGLTPNFKSNFDLAE | acids 15-36: 3xFLAG. |
| | DAKLQLSKDTYDDDLDNLLAQIGDQYADLFLAAKNLSDAILLSDIL | Italics denote |
| | RVNTEITKAPLSASMIKRYDEHHQDLTLLKALVRQQKKKRKVGLP | Neoplasmin NLS. |
| | EKYKEIFFDQSKNGYAGYIDGGASQEEFYKFIKPILEKMDGTEELL | |
| | VKLNREDLLRKQRTFDNGSIPHQIHLGELHAILRRQEDFYPFLKDN | |
| | REKIEKILTFRIPYYVGPLARGNSRFAWMTRKSEETITPWNFEEVV | |
| | DKGASAQSFIERMTNFDKNLPNEKVLPKHSLLYEYFTVYNELTKV | |
| | KYVTEGMRKPAFLSGEQKKAIVDLLFKTNRKVTVKQLKEDYFKKI | |
| | ECFDSVEISGVEDRFNASLGTYHDLLKIIKDKDFLDNEENEDILEDI | |
| | VLTLTLFEDREMIEERLKTYAHLFDDKVMKQLKRRRYTGWGRLS | |
| | RKLINGIRDKQSGKTILDFLKSDGFANRNFMQLIHDDSLTFKEDIQ | |
| | KAQVSGQGDSLHEHIANLAGSPAIKKGILQTVKVVDELVKVMGRH | |
| | KPENIVIEMARENQTTQKGQKNSRERMKRIEEGIKELGSQILKEHP | |
| | VENTQLQNEKLYLYYLQNGRDMINDQELDINRLSDYDVDAIVPQS | |
| | FLKDDSIDNKVLTRSDKNRGKSDNVPSEEVVKKMKNYWRQLLNA | |
| | KLITQRKFDNLTKAERGGLSELDKAGFIKRQLVETRQITKHVAQIL | |
| | DSRMNTKYDENDKLIREVKVITLKSKLVSDFRKDFQFYKVREINNY | |
| | HHAHDAYLNAVVGTALIKKYPKLESEFVYGDYKVYDVRKMIAKSE | |
| | QEIGKATAKYFFYSNIMNFFKTEITLANGEIRKRPLIETNGETGEIV | |
| | WDKGRDFATVRKVLSMPQVNIVKKTEVQTGGFSKESILPKRNSDK | |
| | LIARKKDWDPKKYGGFDSPTVAYSVLVVAKVEKGKSKKLKSVKE | |
| | LLGITIMERSSFEKNPIDFLEAKGYKEVKKDLIIKLPKYSLFELENG | |
| | RKRMLASAGELQKGNELALPSKINNFLYLASHYEKLKGSPEDNEQ | |
| | KQLFVEQHKHYLDEIIEQISEFSKRVILADANLDKVLSAYNKHRDK | |
| | PIREQAENIIHLFTLTNLGAPAAFKYFDTTIDRKRYTSTKEVLDATL | |
| | IHQSITGLYETRIDLSQLGGDGGSGGSGGSGGSGGSASGGGSGGGSK | |
| | RPAATKKAGQAKKKKGGSGSGATNFSLLKQAGDVEENPGPAAA | |
| 83 | MPKKKRKVGGSGGSDYKDHDGDYKDHDIDYKDDDDKGGGSGGGSG | FOG1[1-45]-dCas9: |
| | TMSRRKQSNPRQIKRSLGDMEAREEVQLVGASHMEQKATAPEAPS | location effector |
| | PSTGGSGGSGGSGGSGGSGRPMDKKYSIGLAIGTNSVGWAVITDEY | domain (denoted by |
| | KVPSKKFKVLGNTDRHSIKKNLIGALLFDSGETAEATRLKRTARRR | bold underlined |
| | YTRRKNRICYLQEIFSNEMAKVDDSFFHRLEESFLVEEDKKHERHP | sequence) fused to N- |
| | IFGNIVDEVAYHEKYPTIYHLRKKLVDSTDKADLRLIYLALAHMIK | terminus of dCas9. |
| | FRGHFLIEGDLNPDNSDVDKLFIQLVQTYNQLFEENPINASGVDAK | Underlined denotes |
| | AILSARLSKSRRLENLIAQLPGEKKNGLFGNLIALSLGLTPNFKSNF | (GGS)₅ amino acid |
| | DLAEDAKLQLSKDTYDDDLDNLLAQIGDQYADLFLAAKNLSDAIL | linkers (SEQ ID NO: |
| | LSDILRVNTEITKAPLSASMIKRYDEHHQDLTLLKALVRQQKKKR | 75). Bold denotes |
| | KVGLPEKYKEIFFDQSKNGYAGYIDGGASQEEFYKFIKPILEKMDG | dCas9. Amino acids 2- |
| | TEELLVKLNREDLLRKQRTFDNGSIPHQIHLGELHAILRRQEDFYP | 9: SV40 NLS. Amino |
| | FLKDNREKIEKILTFRIPYYVGPLARGNSRFAWMTRKSEETITPWN | acids 15-36: 3xFLAG. |
| | FEEVVDKGASAQSFIERMTNFDKNLPNEKVLPKHSLLYEYFTVYNE | Italics denote |
| | LTKVKYVTEGMRKPAFLSGEQKKAIVDLLFKTNRKVTVKQLKED | Neoplasmin NLS. |
| | YFKKIECFDSVEISGYEDRFNASLGTYHDLLKIIKDKDFLDNEENED | |
| | ILEDIVLTLTLFEDREMIEERLKTYAHLFDDKVMKQLKRRRYTGW | |
| | GRLSRKLINGIRDKQSGKTILDFLKSDGFANRNFMQLIHDDSLTFK | |
| | EDIQKAQVSGQGDSLHEHIANLAGSPAIKKGILQTVKVVDELVKV | |
| | MGRHKPENIVIEMARENQTTQKGQKNSRERMKRIEEGIKELGSQI | |
| | LKEHPVENTQLQNEKLYLYYLQNGRDMYVDQELDINRLSDYDVD | |
| | AIVPQSFLKDDSIDNKVLTRSDKNRGKSDNVPSEEVVKKMKNYWR | |
| | QLLNAKLITQRKFDNLTKAERGGLSELDKAGFIKRQLVETRQITK | |
| | HVAQILDSRMNTKYDENDKLIREVKVITLKSKLVSDFRKDFQFYKV | |
| | REINNYHHAHDAYLNAVVGTALIKKYPKLESEFVYGDYKVYDVRK | |
| | MIAKSEQEIGKATAKYFFYSNIMNFFKTEITLANGEIRKRPLIETNG | |
| | ETGEIVWDKGRDFATVRKVLSMPQVNIVKKTEVQTGGFSKESILP | |
| | KRNSDKLIARKKDWDPKKYGGFDSPTVAYSVLVVAKVEKGKSKK | |
| | LKSVKELLGITIMERSSFEKNPIDFLEAKGYKEVKKDLIIKLPKYSL | |
| | FELENGRKRMLASAGELQKGNELALPSKYVNFLYLASHYEKLKGS | |
| | PEDNEQKQLFVEQHKHYLDEIIEQISEFSKRVILADANLDKVLSAYN | |
| | KHRDKPIREQAENIIHLFTLTNLGAPAAFKYFDTTIDRKRYTSTKEV | |
| | LDATLIHQSITGLYETRIDLSQLGGDGGSGGSGGSGGSGGSASGGGS | |
| | GGGSKRPAATKKAGQAKKKKGGSGSGATNFSLLKQAGDVEENPGPAAA | |
| 84 | MPKKKRKVGGSGGSDYKDHDGDYKDHDIDYKDDDDKGGGSGGGSG | SUV[SET]-dCas9: |
| | TPRQNLKCVRILKQFHKDLERELLRRHHRSKTPRHLDPSLANYLV | location effector |
| | QKAKQRRALRRWEQELNAKRSHLGRITVENEVDLDGPPRAFVYIN | domain (denoted by |
| | EYRVGEGITLNQVAVGCECQDCLWAPTGGCCPGASLHKFAYNDQ | bold underlined |
| | GQVRLRAGLPIYECNSRCRCGYDCPNRVVQKGIRYDLCIFRTDDG | sequence) fused to N- |
| | RGWGVRTLEKIRKNSFVMEYVGEIITSEEAERRGQIYDRQGATYLF | terminus of dCas9. |
| | DLDYVEDVYTVDAAYYGNISHFVNHSCDPNLQVYNVFIDNLDERLP | Underlined denotes |
| | RIAFFATRTIRAGEELTFDYNMQVDPVDMESTRMDSNFGLAGLPG | (GGS)₅ amino acid |
| | SPKKRVRIECKCGTESCRKYLFSTGGSGGSGGSGGSGGSGRPMDKK | linkers (SEQ ID NO: |
| | YSIGLAIGTNSVGWAVITDEYKVPSKKFKVLGNTDRHSIKKNLIGA | 75). Bold denotes |
| | LLFDSGETAEATRLKRTARRRYTRRKNRICYLQEIFSNEMAKVDDS | dCas9. Amino acids 2- |
| | FFHRLEESFLVEEDKKHERHPIFGNIVDEVAYHEKYPTIYHLRKKL | 9: SV40 NLS. Amino |
| | VDSTDKADLRLIYLALAHMIKFRGHFLIEGDLNPDNSDVDKLFIQL | acids 15-36: 3xFLAG. |
| | VQTYNQLFEENPINASGVDAKAILSARLSKSRRLENLIAQLPGEKK | Italics denote |
| | NGLFGNLIALSLGLTPNFKSNFDLAEDAKLQLSKDTYDDDLDNLLA | Neoplasmin NLS. |
| | QIGDQYADLFLAAKNLSDAILLSDILRVNTEITKAPLSASMIKRYDE | |
| | HHQDLTLLKALVRQQKKKRKVGLPEKYKEIFFDQSKNGYAGYID | |
| | GGASQEEFYKFIKPILEKMDGTEELLVKLNREDLLRKQRTFDNGSI | |
| | PHQIHLGELHAILRRQEDFYPFLKDNREKIEKILTFRIPYYVGPLAR | |
| | GNSRFAWMTRKSEETITPWNFEEVVDKGASAQSFIERMTNFDKNL | |
| | PNEKVLPKHSLLYEYFTVYNELTKVKYVTEGMRKPAFLSGEQKKA | |
| | IVDLLFKTNRKVTVKQLKEDYFKKIECFDSVEISGVEDRFNASLGT | |
| | YHDLLKIIKDKDFLDNEENEDILEDIVLTLTLFEDREMIEERLKTYA | |
| | HLFDDKVMKQLKRRRYTGWGRLSRKLINGIRDKQSGKTILDFLKS | |
| | DGFANRNFMQLIHDDSLTFKEDIQKAQVSGQGDSLHEHIANLAGSP | |
| | AIKKGILQTVKVVDELVKYMGRHKPENIVIEMARENQTTQKGQK | |
| | NSRERMKRIEEGIKELGSQILKEHPVENTQLQNEKLYLYYLQNGR | |
| | DMYVDQELDINRLSDYDVDAIVPQSFLKDDSIDNKYLTRSDKNRGK | |
| | SDNVPSEEVVKKMKNYWRQLLNAKLITQRKFDNLTKAERGGLSE | |
| | LDKAGFIKRQLVETRQITKHVAQILDSRMNTKYDENDKLIREVKVI | |
| | TLKSKLVSDFRKDFQFYKVREINNYHHAHDAYLNAVVGTALIKKY | |
| | PKLESEFVYGDYKVYDVRKMIAKSEQEIGKATAKYFFYSNIMNFFK | |
| | TEITLANGEIRKRPLIETNGETGEIVWDKGRDFATVRKVLSMPQVN | |
| | IYKKTEVQTGGFSKESILPKRNSDKLIARKKDWDPKKYGGFDSPTV | |
| | AYSVLVVAKVEKGKSKKLKSVKELLGITIMERSSFEKNPIDFLEAK | |
| | GYKEVKKDLIIKLPKYSLFELENGRKRMLASAGELQKGNELALPS | |
| | KYVNFLYLASHYEKLKGSPEDNEQKQLFVEQHKHYLDEIIEQISEF | |
| | SKRVILADANLDKVLSAYNKHRDKPIREQAENIIHLFTLTNLGAPA | |
| | AFKYFDTTIDRKRYTSTKEVLDATLIHQSITGLYETRIDLSQLGGDG | |
| | GSGGSGGSGGSGGSASGGGSGGGSKRPAATKKAGQAKKKKGGSGSGAT | |
| | NFSLLKQAGDVEENPGPAAA | |
| 85 | MPKKKRKVGGSGGSDYKDHDGDYKDHDIDYKDDDDKGGGSGGGSG | G9A[SET]-dCas9: |
| | TGSAAIAEVLLNARCDLHAVNYHGDTPLHIAARESYHDCVLLFLSR | location effector |
| | GANPELRNKEGDTAWDLTPERSDVWFALQLNRKLRLGVGNRAIR | domain (denoted by |
| | TEKIICRDVARGYENVPIPCVNGVDGEPCPEDYKYISENCETSTMNI | bold underlined |
| | DRNITHLQHCTCVDDCSSSNCLCGQLSIRCWYDKDGRLLQEFNKIE | sequence) fused to N- |
| | PPLIFECNQACSCWRNCKNRVVQSGIKVRLQLYRTAKMGWGVRA | terminus of dCas9. |
| | LQTIPQGTFICEYVGELISDAEADVREDDSYLFDLDNKDGEVYCIDA | Underlined denotes |
| | RYYGNISRFINHLCDPNIIPVRVFMLHQDLRFPRIAFFSSRDIRTGEE | (GGS)₅ amino acid |
| | LGFDYGDRFWDIKSKYFTCQCGSEKCKHSAEAIALEQSRLARLDP | linkers (SEQ ID NO: |
| | HPELLPELGSLPPVNSTGGSGGSGGSGGSGGSGRPMDKKYSIGLAIG | 75). Bold denotes |
| | TNSVGWAVITDEYKVPSKKFKVLGNTDRHSIKKNLIGALLFDSGET | dCas9. Amino acids 2- |
| | AEATRLKRTARRRYTRRKNRICYLQEIFSNEMAKVDDSFFHRLEES | 9: SV40 NLS. Amino |
| | FLVEEDKKHERHPIFGNIVDEVAYHEKYPTIYHLRKKLVDSTDKAD | acids 15-36: 3xFLAG. |
| | LRLIYLALAHMIKFRGHFLIEGDLNPDNSDVDKLFIQLVQTYNQLF | Italics denote |
| | EENPINASGVDAKAILSARLSKSRRLENLIAQLPGEKKNGLFGNLIA | Neoplasmin NLS. |
| | LSLGLTPNFKSNFDLAEDAKLQLSKDTYDDDLDNLLAQIGDQYAD | |
| | LFLAAKNLSDAILLSDILRVNTEITKAPLSASMIKRYDEHHQDLTLL | |
| | KALVRQQKKKRKVGLPEKYKEIFFDQSKNGYAGYIDGGASQEEFY | |
| | KFIKPILEKMDGTEELLVKLNREDLLRKQRTFDNGSIPHQIHLGEL | |
| | HAILRRQEDFYPFLKDNREKIEKILTFRIPYYVGPLARGNSRFAWM | |
| | TRKSEETITPWNFEEVVDKGASAQSFIERMTNFDKNLPNEKVLPKH | |
| | SLLYEYFTVYNELTKVKYVTEGMRKPAFLSGEQKKAIVDLLFKTN | |
| | RKVTVKQLKEDYFKKIECFDSVEISGVEDRFNASLGTYHDLLKIIK | |
| | DKDFLDNEENEDILEDIVLTLTLFEDREMIEERLKTYAHLFDDKVM | |
| | KQLKRRRYTGWGRLSRKLINGIRDKQSGKTILDFLKSDGFANRNF | |
| | MQLIHDDSLTFKEDIQKAQVSGQGDSLHEHIANLAGSPAIKKGILQ | |
| | TVKVVDELVKVMGRHKPENIVIEMARENQTTQKGQKNSRERMKR | |
| | IEEGIKELGSQILKEHPVENTQLQNEKLYLYYLQNGRDMYVDQEL | |
| | DINRLSDYDVDAIVPQSFLKDDSIDNKVLTRSDKNRGKSDNVPSEEV | |
| | VKKMKNYWRQLLNAKLITQRKFDNLTKAERGGLSELDKAGFIKR | |
| | QLVETRQITKHVAQILDSRMNTKYDENDKLIREVKVITLKSKLVSD | |
| | FRKDFQFYKVREINNYHHAHDAYLNAVVGTALIKKYPKLESEFVY | |
| | GDYKVYDVRKMIAKSEQEIGKATAKYFFYSNIMNFFKTEITLANGE | |
| | IRKRPLIETNGETGEIVWDKGRDFATVRKVLSMPQVNIVKKTEVQ | |
| | TGGFSKESILPKRNSDKLIARKKDWDPKKYGGFDSPTVAYSVLVVA | |
| | KVEKGKSKKLKSVKELLGITIMERSSFEKNPIDFLEAKGYKEVKKD | |
| | LIIKLPKYSLFELENGRKRMLASAGELQKGNELALPSKYVNFLYLA | |
| | SHYEKLKGSPEDNEQKQLFVEQHKHYLDEIIEQISEFSKRVILADAN | |
| | LDKVLSAYNKHRDKPIREQAENIIHLFTLTNLGAPAAFKYFDTTIDR | |
| | KRYTSTKEVLDATLIHQSITGLYETRIDLSQLGGDGGSGGSGGSGGS | |
| | GGSASGGGSGGGSKRPAATKKAGQAKKKKGGSGSGATNFSLLKQAGD | |
| | VEENPGPAAA | |
| 86 | MPKKKRKVGGSGGSDYKDHDGDYKDHDIDYKDDDDKGGGSGGGSG | DNMT3A-dCas9: |
| | TRAPSRLQMFFANNHDQEFDPPKVYPPVPAEKRKPIRVLSLFDGIA | location effector |
| | TGLLVLKDLGIQVDRYIASEVCEDSITVGMVRHQGKIMYVGDVRS | domain (denoted by |
| | VTQKHIQEWGPFDLVIGGSPCNDLSIVNPARKGLYEGTGRLFFEFY | bold underlined |
| | RLLHDARPKEGDDRPFFWLFENVVAMGVSDKRDISRFLESNPVMI | sequence) fused to N- |
| | DAKEVSAAHRARYFWGNLPGMNRPLASTVNDKLELQECLEHGRI | terminus of dCas9. |
| | AKFSKVRTITTRSNSIKQGKDQHFPVFMNEKEDILWCTEMERVFG | Underlined denotes |
| | FPVHYTDVSNMSRLARQRLLGRSWSVPVIRHLFAPLKEYFACVSTG | (GGS)₅ amino acid |
| | GSGGSGGSGGSGGSGRPMDKKYSIGLAIGTNSVGWAVITDEYKVPS | linkers (SEQ ID NO: |
| | KKFKVLGNTDRHSIKKNLIGALLFDSGETAEATRLKRTARRRYTR | 75). Bold denotes |
| | RKNRICYLQEIFSNEMAKVDDSFFHRLEESFLVEEDKKHERHPIFG | dCas9. Amino acids 2- |
| | NIVDEVAYHEKYPTIYHLRKKLVDSTDKADLRLIYLALAHMIKFRG | 9: SV40 NLS. Amino |
| | HFLIEGDLNPDNSDVDKLFIQLVQTYNQLFEENPINASGVDAKAILS | acids 15-36: 3xFLAG. |
| | ARLSKSRRLENLIAQLPGEKKNGLFGNLIALSLGLTPNFKSNFDLA | Italics denote |
| | EDAKLQLSKDTYDDDLDNLLAQIGDQYADLFLAAKNLSDAILLSDI | Neoplasmin NLS. |
| | LRVNTEITKAPLSASMIKRYDEHHQDLTLLKALVRQQKKKRKVGL | |
| | PEKYKEIFFDQSKNGYAGYIDGGASQEEFYKFIKPILEKMDGTEEL | |
| | LVKLNREDLLRKQRTFDNGSIPHQIHLGELHAILRRQEDFYPFLKD | |
| | NREKIEKILTFRIPYYVNGPLARGNSRFAWMTRKSEETITPWNFEEV | |
| | VDKGASAQSFIERMTNFDKNLPNEKVLPKHSLLYEYFTVYNELTK | |
| | VKYVTEGMRKPAFLSGEQKKAIVDLLFKTNRKVTVKQLKEDYFK | |
| | KIECFDSVEISGVEDRFNASLGTYHDLLKIIKDKDFLDNEENEDILE | |
| | DIVLTLTLFEDREMIEERLKTYAHLFDDKVMKQLKRRRYTGWGR | |
| | LSRKLINGIRDKQSGKTILDFLKSDGFANRNFMQLIHDDSLTFKEDI | |
| | QKAQVSGQGDSLHEHIANLAGSPAIKKGILQTVKVVDELVKVMGR | |
| | HKPENIVIEMARENQTTQKGQKNSRERMKRIEEGIKELGSQILKEH | |
| | PVENTQLQNEKLYLYYLQNGRDMYVDQELDINRLSDYDVDAIVPQ | |
| | SFLKDDSIDNKVLTRSDKNRGKSDNVPSEEVVKKMKNYWRQLLNA | |
| | KLITQRKFDNLTKAERGGLSELDKAGFIKRQLVETRQITKHVAQIL | |
| | DSRMNTKYDENDKLIREVKVITLKSKLVSDFRKDFQFYKVREINNY | |
| | HHAHDAYLNAVVGTALIKKYPKLESEFVYGDYKVYDVRKMIAKSE | |
| | QEIGKATAKYFFYSNIMNFFKTEITLANGEIRKRPLIETNGETGEIV | |
| | WDKGRDFATVRKVLSMPQVNIVKKTEVQTGGFSKESILPKRNSDK | |
| | LIARKKDWDPKKYGGFDSPTVAYSVLVVAKVEKGKSKKLKSVKE | |
| | LLGITIMERSSFEKNPIDFLEAKGYKEVKKDLIIKLPKYSLFELENG | |
| | RKRMLASAGELQKGNELALPSKYVNFLYLASHYEKLKGSPEDNEQ | |
| | KQLFVEQHKHYLDEIIEQISEFSKRVILADANLDKVLSAYNKHRDK | |
| | PIREQAENIIHLFTLTNLGAPAAFKYFDTTIDRKRYTSTKEVLDATL | |
| | IHQSITGLYETRIDLSQLGGDGGSGGSGGSGGSGGSASGGGSGGGSK | |
| | RPAATKKAGQAKKKKGGSGSGATNFSLLKQAGDVEENPGPAAA | |
| 87 | MPKKKRKVGGSGGSDYKDHDGDYKDHDIDYKDDDDKGGGSGGGSG | KRAB-dCas9: location |
| | TTLVTFKDVFVDFTREEWKLLDTAQQIVYRNVMLENYKNLVSLGY | effector domain |
| | QLTKPDVILRLEKGEEPWLVEREIHQETHPSTGGSGGSGGSGGSGG | (denoted by bold |
| | SGRPMDKKYSIGLAIGTNSVGWAVITDEYKVPSKKFKVLGNTDRHS | underlined sequence) |
| | IKKNLIGALLFDSGETAEATRLKRTARRRYTRRKNRICYLQEIFSNE | fused to N-terminus of |
| | MAKVDDSFFHRLEESFLVEEDKKHERHPIFGNIVDEVAYHEKYPTI | dCas9. Underlined |
| | YHLRKKLVDSTDKADLRLIYLALAHMIKFRGHFLIEGDLNPDNSDV | denotes (GGS)₅ amino |
| | DKLFIQLVQTYNQLFEENPINASGVDAKAILSARLSKSRRLENLIAQ | acid linkers (SEQ ID |
| | LPGEKKNGLFGNLIALSLGLTPNFKSNFDLAEDAKLQLSKDTYDDD | NO: 75). Bold denotes |
| | LDNLLAQIGDQYADLFLAAKNLSDAILLSDILRVNTEITKAPLSASM | dCas9. Amino acids 2- |
| | IKRYDEHHQDLTLLKALVRQQKKKRKVGLPEKYKEIFFDQSKNG | 9: SV40 NLS. Amino |
| | YAGYIDGGASQEEFYKFIKPILEKMDGTEELLVKLNREDLLRKQR | acids 15-36: 3xFLAG. |
| | TFDNGSIPHQIHLGELHAILRRQEDFYPFLKDNREKIEKILTFRIPYY | Italics denote |
| | VGPLARGNSRFAWMTRKSEETITPWNFEEVVDKGASAQSFIERMT | Neoplasmin NLS. |
| | NFDKNLPNEKVLPKHSLLYEYFTVYNELTKVKYVTEGMRKPAFLS | |
| | GEQKKAIVDLLFKTNRKVTVKQLKEDYFKKIECFDSVEISGVEDRF | |
| | NASLGTYHDLLKIIKDKDFLDNEENEDILEDIVLTLTLFEDREMIEE | |
| | RLKTYAHLFDDKVMKQLKRRRYTGWGRLSRKLINGIRDKQSGKT | |
| | ILDFLKSDGFANRNFMQLIHDDSLTFKEDIQKAQVSGQGDSLHEHI | |
| | ANLAGSPAIKKGILQTVKVVDELVKVMGRHKPENIVIEMARENQT | |
| | TQKGQKNSRERMKRIEEGIKELGSQILKEHPVENTQLQNEKLYLY | |
| | YLQNGRDMYVDQELDINRLSDYDVDAIVPQSFLKDDSIDNKVLTRS | |
| | DKNRGKSDNVPSEEVVKKMKNYWRQLLNAKLITQRKFDNLTKAE | |
| | RGGLSELDKAGFIKRQLVETRQITKHVAQILDSRMNTKYDENDKLI | |
| | REVKVITLKSKLVSDFRKDFQFYKVREINNYHHAHDAYLNAVVGT | |
| | ALIKKYPKLESEFVYGDYKVYDVRKMIAKSEQEIGKATAKYFFYSN | |
| | IMNFFKTEITLANGEIRKRPLIETNGETGEIVWDKGRDFATVRKVL | |
| | SMPQVNIVKKTEVQTGGFSKESILPKRNSDKLIARKKDWDPKKYG | |
| | GFDSPTVAYSVLVVAKVEKGKSKKLKSVKELLGITIMERSSFEKNP | |
| | IDFLEAKGYKEVKKDLIIKLPKYSLFELENGRKRMLASAGELQKG | |
| | NELALPSKYVNFLYLASHYEKLKGSPEDNEQKQLFVEQHKHYLDE | |
| | IIEQISEFSKRVILADANLDKVLSAYNKHRDKPIREQAENIIHLFTLT | |
| | NLGAPAAFKYFDTTIDRKRYTSTKEVLDATLIHQSITGLYETRIDLS | |
| | QLGGDGGSGGSGGSGGSGGSASGGGSGGGSKRPAATKKAGQAKKKKG | |
| | GSGSGATNFSLLKQAGDVEENPGPAAA | |
| 88 | MPKKKRKVGGSGGSDYKDHDGDYKDHDIDYKDDDDKGGGSGGGSG | dCas9-Ezh2[FL]: |
| | TGGSGGSGGSGGSGGSGRPMDKKYSIGLAIGTNSVGWAVITDEYKV | location effector |
| | PSKKFKVLGNTDRHSIKKNLIGALLFDSGETAEATRLKRTARRRYT | domain (denoted by |
| | RRKNRICYLQEIFSNEMAKVDDSFFHRLEESFLVEEDKKHERHPIF | bold underlined |
| | GNIVDEVAYHEKYPTIYHLRKKLVDSTDKADLRLIYLALAHMIKFR | sequence) fused to C- |
| | GHFLIEGDLNPDNSDVDKLFIQLVQTYNQLFEENPINASGVDAKAIL | terminus of dCas9. |
| | SARLSKSRRLENLIAQLPGEKKNGLFGNLIALSLGLTPNFKSNFDLA | Underlined denotes |
| | EDAKLQLSKDTYDDDLDNLLAQIGDQYADLFLAAKNLSDAILLSDI | (GGS)₅ amino acid |
| | LRVNTEITKAPLSASMIKRYDEHHQDLTLLKALVRQQKKKRKVGL | linkers (SEQ ID NO: |
| | PEKYKEIFFDQSKNGYAGYIDGGASQEEFYKFIKPILEKMDGTEEL | 75). Bold denotes |
| | LVKLNREDLLRKQRTFDNGSIPHQIHLGELHAILRRQEDFYPFLKD | dCas9. Amino acids 2- |
| | NREKIEKILTFRIPYYVGPLARGNSRFAWMTRKSEETITPWNFEEV | 9: SV40 NLS. Amino |
| | VDKGASAQSFIERMTNFDKNLPNEKVLPKHSLLYEYFTVYNELTK | acids 15-36: 3xFLAG. |
| | VKYVTEGMRKPAFLSGEQKKAIVDLLFKTNRKVTVKQLKEDYFK | Italics denote |
| | KIECFDSVEISGVEDRFNASLGTYHDLLKIIKDKDFLDNEENEDILE | Neoplasmin NLS. |
| | DIVLTLTLFEDREMIEERLKTYAHLFDDKVMKQLKRRRYTGWGR | |
| | LSRKLINGIRDKQSGKTILDFLKSDGFANRNFMQLIHDDSLTFKEDI | |
| | QKAQVSGQGDSLHEHIANLAGSPAIKKGILQTVKVVDELVKVMGR | |
| | HKPENIVIEMARENQTTQKGQKNSRERMKRIEEGIKELGSQILKEH | |
| | PVENTQLQNEKLYLYYLQNGRDMINDQELDINRLSDYDVDAIVPQ | |
| | SFLKDDSIDNKVLTRSDKNRGKSDNVPSEEVVKKMKNYWRQLLNA | |
| | KLITQRKFDNLTKAERGGLSELDKAGFIKRQLVETRQITKHVAQIL | |
| | DSRMNTKYDENDKLIREVKVITLKSKLVSDFRKDFQFYKVREINNY | |
| | HHAHDAYLNAVVGTALIKKYPKLESEFVYGDYKVYDVRKMIAKSE | |
| | QEIGKATAKYFFYSNIMNFFKTEITLANGEIRKRPLIETNGETGEIV | |
| | WDKGRDFATVRKVLSMPQVNIVKKTEVQTGGFSKESILPKRNSDK | |
| | LIARKKDWDPKKYGGFDSPTVAYSVLVVAKVEKGKSKKLKSVKE | |
| | LLGITIMERSSFEKNPIDFLEAKGYKEVKKDLIIKLPKYSLFELENG | |
| | RKRMLASAGELQKGNELALPSKYVNFLYLASHYEKLKGSPEDNEQ | |
| | KQLFVEQHKHYLDEIIEQISEFSKRVILADANLDKVLSAYNKHRDK | |
| | PIREQAENIIHLFTLTNLGAPAAFKYFDTTIDRKRYTSTKEVLDATL | |
| | IHQSITGLYETRIDLSQLGGDGGSGGSGGSGGSGGSASMGQTGKKS | |
| | EKGPVCWRKRVKSEYMRLRQLKRFRRADEVKTMFSSNRQKILER | |
| | TETLNQEWKQRRIQPVHIMTSVSSLRGTRECSVTSDLDFPAQVIPL | |
| | KTLNAVASVPIMYSWSPLQQNFMVEDETVLHNIPYMGDEVLDQDG | |
| | TFIEELIKNYDGKVHGDRECGFINDEIFVELVNALGQYNDDDDDDD | |
| | GDDPDEREEKQKDLEDNRDDKETCPPRKFPADKIFEAISSMFPDKG | |
| | TAEELKEKYKELTEQQLPGALPPECTPNIDGPNAKSVQREQSLHSF | |
| | HTLFCRRCFKYDCFLHPFHATPNTYKRKNTETALDNKPCGPQCYQ | |
| | HLEGAKEFAAALTAERIKTPPKRPGGRRRGRLPNNSSRPSTPTISVL | |
| | ESKDTDSDREAGTETGGENNDKEEEEKKDETSSSSEANSRCQTPIK | |
| | MKPNIEPPENVEWSGAEASMFRVLIGTYYDNFCAIARLIGTKTCRQ | |
| | VYEFRVKESSIIAPVPTEDVDTPPRKKKRKHRLWAAHCRKIQLKK | |
| | DGSSNHVYNYQPCDHPRQPCDSSCPCVIAQNFCEKFCQCSSECQNR | |
| | FPGCRCKAQCNTKQCPCYLAVRECDPDLCLTCGAADHWDSKNVS | |
| | CKNCSIQRGSKKHLLLAPSDVAGWGIFIKDPVQKNEFISEYCGEIIS | |
| | QDEADRRGKVYDKYMCSFLFNLNNDFVVDATRKGNKIRFANHSV | |
| | NPNCYAKVMMVNGDHRIGIFAKRAIQTGEELFFDYRYSQADALKY | |
| | VGIEREMEIPTSGGGSGGGSKRPAATKKAGQAKKKKGGSGSGATNFSL | |
| | LKQAGDVEENPGPAAA | |
| 89 | MPKKKRKVGGSGGSDYKDHDGDYKDHDIDYKDDDDKGGGSGGGSG | dCas9-Ezh2[SET]: |
| | TGGSGGSGGSGGSGGSGRPMDKKYSIGLAIGTNSVGWAVITDEYKV | location effector |
| | PSKKFKVLGNTDRHSIKKNLIGALLFDSGETAEATRLKRTARRRYT | domain (denoted by |
| | RRKNRICYLQEIFSNEMAKVDDSFFHRLEESFLVEEDKKHERHPIF | bold underlined |
| | GNIVDEVAYHEKYPTIYHLRKKLVDSTDKADLRLIYLALAHMIKFR | sequence) fused to C- |
| | GHFLIEGDLNPDNSDVDKLFIQLVQTYNQLFEENPINASGVDAKAIL | terminus of dCas9. |
| | SARLSKSRRLENLIAQLPGEKKNGLFGNLIALSLGLTPNFKSNFDLA | Underlined denotes |
| | EDAKLQLSKDTYDDDLDNLLAQIGDQYADLFLAAKNLSDAILLSDI | (GGS)₅ amino acid |
| | LRVNTEITKAPLSASMIKRYDEHHQDLTLLKALVRQQKKKRKVGL | linkers (SEQ ID NO: |
| | PEKYKEIFFDQSKNGYAGYIDGGASQEEFYKFIKPILEKMDGTEEL | 75). Bold denotes |
| | LVKLNREDLLRKQRTFDNGSIPHQIHLGELHAILRRQEDFYPFLKD | dCas9. Amino acids 2- |
| | NREKIEKILTFRIPYYVGPLARGNSRFAWMTRKSEETITPWNFEEV | 9: SV40 NLS. Amino |
| | VDKGASAQSFIERMTNFDKNLPNEKVLPKHSLLYEYFTVYNELTK | acids 15-36: 3xFLAG. |
| | VKYVTEGMRKPAFLSGEQKKAIVDLLFKTNRKVTVKQLKEDYFK | Italics denote |
| | KIECFDSVEISGVEDRFNASLGTYHDLLKIIKDKDFLDNEENEDILE | Neoplasmin NLS. |
| | DIVLTLTLFEDREMIEERLKTYAHLFDDKVMKQLKRRRYTGWGR | |
| | LSRKLINGIRDKQSGKTILDFLKSDGFANRNFMQLIHDDSLTFKEDI | |
| | QKAQVSGQGDSLHEHIANLAGSPAIKKGILQTVKVVDELVKVMGR | |
| | HKPENIVIEMARENQTTQKGQKNSRERMKRIEEGIKELGSQILKEH | |
| | PVENTQLQNEKLYLYYLQNGRDMYVDQELDINRLSDYDVDAIVPQ | |
| | SFLKDDSIDNKVLTRSDKNRGKSDNVPSEEVVKKMKNYWRQLLNA | |
| | KLITQRKFDNLTKAERGGLSELDKAGFIKRQLVETRQITKHVAQIL | |
| | DSRMNTKYDENDKLIREVKVITLKSKLVSDFRKDFQFYKVREINNY | |
| | HHAHDAYLNAVVGTALIKKYPKLESEFVYGDYKVYDVRKMIAKSE | |
| | QEIGKATAKYFFYSNIMNFFKTEITLANGEIRKRPLIETNGETGEIV | |
| | WDKGRDFATVRKVLSMPQVNIVKKTEVQTGGFSKESILPKRNSDK | |
| | LIARKKDWDPKKYGGFDSPTVAYSVLVVAKVEKGKSKKLKSVKE | |
| | LLGITIMERSSFEKNPIDFLEAKGYKEVKKDLIIKLPKYSLFELENG | |
| | RKRMLASAGELQKGNELALPSKYVNFLYLASHYEKLKGSPEDNEQ | |
| | KQLFVEQHKHYLDEIIEQISEFSKRVILADANLDKVLSAYNKHRDK | |
| | PIREQAENIIHLFTLTNLGAPAAFKYFDTTIDRKRYTSTKEVLDATL | |
| | IHQSITGLYETRIDLSQLGGDGGSGGSGGSGGSGGSASTEDVDTPPR | |
| | KKKRKHRLWAAHCRKIQLKKDGSSNHVYNYQPCDHPRQPCDSSC | |
| | PCVIAQNFCEKFCQCSSECQNRFPGCRCKAQCNTKQCPCYLAVRE | |
| | CDPDLCLTCGAADHWDSKNVSCKNCSIQRGSKKHLLLAPSDVAG | |
| | WGIFIKDPVQKNEFISEYCGEIISQDEADRRGKVYDKYMCSFLFNL | |
| | NNDFVVDATRKGNKIRFANHSVNPNCYAKVMMVNGDHRIGIFAK | |
| | RAIQTGEELFFDYRYSQADALKYVGIEREMEIPTSGGGSGGGSKRPA | |
| | ATKKAGQAKKKKGGSGSGATNFSLLKQAGDVEENPGPAAA | |
| 90 | MPKKKRKVGGSGGSDYKDHDGDYKDHDIDYKDDDDKGGGSGGGSG | dCas9-FOG1[1-45]: |
| | TGGSGGSGGSGGSGGSGRPMDKKYSIGLAIGTNSVGWAVITDEYKV | location effector |
| | PSKKFKVLGNTDRHSIKKNLIGALLFDSGETAEATRLKRTARRRYT | domain (denoted by |
| | RRKNRICYLQEIFSNEMAKVDDSFFHRLEESFLVEEDKKHERHPIF | bold underlined |
| | GNIVDEVAYHEKYPTIYHLRKKLVDSTDKADLRLIYLALAHMIKFR | sequence) fused to C- |
| | GHFLIEGDLNPDNSDVDKLFIQLVQTYNQLFEENPINASGVDAKAIL | terminus of dCas9. |
| | SARLSKSRRLENLIAQLPGEKKNGLFGNLIALSLGLTPNFKSNFDLA | Underlined denotes |
| | EDAKLQLSKDTYDDDLDNLLAQIGDQYADLFLAAKNLSDAILLSDI | (GGS)₅ amino acid |
| | LRVNTEITKAPLSASMIKRYDEHHQDLTLLKALVRQQKKKRKVGL | linkers (SEQ ID NO: |
| | PEKYKEIFFDQSKNGYAGYIDGGASQEEFYKFIKPILEKMDGTEEL | 75). Bold denotes |
| | LVKLNREDLLRKQRTFDNGSIPHQIHLGELHAILRRQEDFYPFLKD | dCas9. Amino acids 2- |
| | NREKIEKILTFRIPYYVGPLARGNSRFAWMTRKSEETITPWNFEEV | 9: SV40 NLS. Amino |
| | VDKGASAQSFIERMTNFDKNLPNEKVLPKHSLLYEYFTVYNELTK | acids 15-36: 3xFLAG. |
| | VKYVTEGMRKPAFLSGEQKKAIVDLLFKTNRKVTVKQLKEDYFK | Italics denote |
| | KIECFDSVEISGVEDRFNASLGTYHDLLKIIKDKDFLDNEENEDILE | Neoplasmin NLS. |
| | DIVLTLTLFEDREMIEERLKTYAHLFDDKVMKQLKRRRYTGWGR | |
| | LSRKLINGIRDKQSGKTILDFLKSDGFANRNFMQLIHDDSLTFKEDI | |
| | QKAQVSGQGDSLHEHIANLAGSPAIKKGILQTVKVVDELVKVMGR | |
| | HKPENIVIEMARENQTTQKGQKNSRERMKRIEEGIKELGSQILKEH | |
| | PVENTQLQNEKLYLYYLQNGRDMYVDQELDINRLSDYDVDAIVPQ | |
| | SFLKDDSIDNKVLTRSDKNRGKSDNVPSEEVVKKMKNYWRQLLNA | |
| | KLITQRKFDNLTKAERGGLSELDKAGFIKRQLVETRQITKHVAQIL | |
| | DSRMNTKYDENDKLIREVKVITLKSKLVSDFRKDFQFYKVREINNY | |
| | HHAHDAYLNAVVGTALIKKYPKLESEFVYGDYKVYDVRKMIAKSE | |
| | QEIGKATAKYFFYSNIMNFFKTEITLANGEIRKRPLIETNGETGEIV | |
| | WDKGRDFATVRKVLSMPQVNIVKKTEVQTGGFSKESILPKRNSDK | |
| | LIARKKDWDPKKYGGFDSPTVAYSVLVVAKVEKGKSKKLKSVKE | |
| | LLGITIMERSSFEKNPIDFLEAKGYKEVKKDLIIKLPKYSLFELENG | |
| | RKRMLASAGELQKGNELALPSKYVNFLYLASHYEKLKGSPEDNEQ | |
| | KQLFVEQHKHYLDEIIEQISEFSKRVILADANLDKVLSAYNKHRDK | |
| | PIREQAENIIHLFTLTNLGAPAAFKYFDTTIDRKRYTSTKEVLDATL | |
| | IHQSITGLYETRIDLSQLGGDGGSGGSGGSGGSGGSASMSRRKQSNP | |
| | RQIKRSLGDMEAREEVQLVGASHMEQKATAPEAPSPTSGGGSGGG | |
| | SKRPAATKKAGQAKKKKGGSGSGATNFSLLKQAGDVEENPGPAAA | |
| 91 | MPKKKRKVGGSGGSDYKDHDGDYKDHDIDYKDDDDKGGGSGGGSG | dCas9-SUV[SET]: |
| | TGGSGGSGGSGGSGGSGRPMDKKYSIGLAIGTNSVGWAVITDEYKV | location effector |
| | PSKKFKVLGNTDRHSIKKNLIGALLFDSGETAEATRLKRTARRRYT | domain (denoted by |
| | RRKNRICYLQEIFSNEMAKVDDSFFHRLEESFLVEEDKKHERHPIF | bold underlined |
| | GNIVDEVAYHEKYPTIYHLRKKLVDSTDKADLRLIYLALAHMIKFR | sequence) fused to C- |
| | GHFLIEGDLNPDNSDVDKLFIQLVQTYNQLFEENPINASGVDAKAIL | terminus of dCas9. |
| | SARLSKSRRLENLIAQLPGEKKNGLFGNLIALSLGLTPNFKSNFDLA | Underlined denotes |
| | EDAKLQLSKDTYDDDLDNLLAQIGDQYADLFLAAKNLSDAILLSDI | (GGS)₅ amino acid |
| | LRVNTEITKAPLSASMIKRYDEHHQDLTLLKALVRQQKKKRKVGL | linkers (SEQ ID NO: |
| | PEKYKEIFFDQSKNGYAGYIDGGASQEEFYKFIKPILEKMDGTEEL | 75). Bold denotes |
| | LVKLNREDLLRKQRTFDNGSIPHQIHLGELHAILRRQEDFYPFLKD | dCas9. Amino acids 2- |
| | NREKIEKILTFRIPYYVGPLARGNSRFAWMTRKSEETITPWNFEEV | 9: SV40 NLS. Amino |
| | VDKGASAQSFIERMTNFDKNLPNEKVLPKHSLLYEYFTVYNELTK | acids 15-36: 3xFLAG. |
| | VKYVTEGMRKPAFLSGEQKKAIVDLLFKTNRKVTVKQLKEDYFK | Italics denote |
| | KIECFDSVEISGVEDRFNASLGTYHDLLKIIKDKDFLDNEENEDILE | Neoplasmin NLS. |
| | DIVLTLTLFEDREMIEERLKTYAHLFDDKVMKQLKRRRYTGWGR | |
| | LSRKLINGIRDKQSGKTILDFLKSDGFANRNFMQLIHDDSLTFKEDI | |
| | QKAQVSGQGDSLHEHIANLAGSPAIKKGILQTVKVVDELVKVMGR | |
| | HKPENIVIEMARENQTTQKGQKNSRERMKRIEEGIKELGSQILKEH | |
| | PVENTQLQNEKLYLYYLQNGRDMYVDQELDINRLSDYDVDAIVPQ | |
| | SFLKDDSIDNKVLTRSDKNRGKSDNVPSEEVVKKMKNYWRQLLNA | |
| | KLITQRKFDNLTKAERGGLSELDKAGFIKRQLVETRQITKHVAQIL | |
| | DSRMNTKYDENDKLIREVKVITLKSKLVSDFRKDFQFYKVREINNY | |
| | HHAHDAYLNAVVGTALIKKYPKLESEFVYGDYKVYDVRKMIAKSE | |
| | QEIGKATAKYFFYSNIMNFFKTEITLANGEIRKRPLIETNGETGEIV | |
| | WDKGRDFATVRKVLSMPQVNIVKKTEVQTGGFSKESILPKRNSDK | |
| | LIARKKDWDPKKYGGFDSPTVAYSVLVVAKVEKGKSKKLKSVKE | |
| | LLGITIMERSSFEKNPIDFLEAKGYKEVKKDLIIKLPKYSLFELENG | |
| | RKRMLASAGELQKGNELALPSKYVNFLYLASHYEKLKGSPEDNEQ | |
| | KQLFVEQHKHYLDEIIEQISEFSKRVILADANLDKVLSAYNKHRDK | |
| | PIREQAENIIHLFTLTNLGAPAAFKYFDTTIDRKRYTSTKEVLDATL | |
| | IHQSITGLYETRIDLSQLGGDGGSGGSGGSGGSGGSASPRQNLKCVR | |
| | ILKQFHKDLERELLRRHHRSKTPRHLDPSLANYLVQKAKQRRALR | |
| | RWEQELNAKRSHLGRITVENEVDLDGPPRAFVYINEYRVGEGITLN | |
| | QVAVGCECQDCLWAPTGGCCPGASLHKFAYNDQGQVRLRAGLPI | |
| | YECNSRCRCGYDCPNRVVQKGIRYDLCIFRTDDGRGWGVRTLEKI | |
| | RKNSFVMEYVGEIITSEEAERRGQIYDRQGATYLFDLDYVEDVYTV | |
| | DAAYYGNISHFVNHSCDPNLQVYNVFIDNLDERLPRIAFFATRTIRA | |
| | GEELTFDYNMQVDPVDMESTRMDSNFGLAGLPGSPKKRVRIECKC | |
| | GTESCRKYLFTSGGGSGGGSKRPAATKKAGQAKKKKGGSGSGATNFSL | |
| | LKQAGDVEENPGPAAA | |
| 92 | MPKKKRKVGGSGGSDYKDHDGDYKDHDIDYKDDDDKGGGSGGGSG | dCas9-G9A[SET]: |
| | TGGSGGSGGSGGSGGSGRPMDKKYSIGLAIGTNSVGWAVITDEYKV | location effector |
| | PSKKFKVLGNTDRHSIKKNLIGALLFDSGETAEATRLKRTARRRYT | domain (denoted by |
| | RRKNRICYLQEIFSNEMAKVDDSFFHRLEESFLVEEDKKHERHPIF | bold underlined |
| | GNIVDEVAYHEKYPTIYHLRKKLVDSTDKADLRLIYLALAHMIKFR | sequence) fused to C- |
| | GHFLIEGDLNPDNSDVDKLFIQLVQTYNQLFEENPINASGVDAKAIL | terminus of dCas9. |
| | SARLSKSRRLENLIAQLPGEKKNGLFGNLIALSLGLTPNFKSNFDLA | Underlined denotes |
| | EDAKLQLSKDTYDDDLDNLLAQIGDQYADLFLAAKNLSDAILLSDI | (GGS)₅ amino acid |
| | LRVNTEITKAPLSASMIKRYDEHHQDLTLLKALVRQQKKKRKVGL | linkers (SEQ ID NO: |
| | PEKYKEIFFDQSKNGYAGYIDGGASQEEFYKFIKPILEKMDGTEEL | 75). Bold denotes |
| | LVKLNREDLLRKQRTFDNGSIPHQIHLGELHAILRRQEDFYPFLKD | dCas9. Amino acids 2- |
| | NREKIEKILTFRIPYYVGPLARGNSRFAWMTRKSEETITPWNFEEV | 9: SV40 NLS. Amino |
| | VDKGASAQSFIERMTNFDKNLPNEKVLPKHSLLYEYFTVYNELTK | acids 15-36: 3xFLAG. |
| | VKYVTEGMRKPAFLSGEQKKAIVDLLFKTNRKVTVKQLKEDYFK | Italics denote |
| | KIECFDSVEISGVEDRFNASLGTYHDLLKIIKDKDFLDNEENEDILE | Neoplasmin NLS. |
| | DIVLTLTLFEDREMIEERLKTYAHLFDDKVMKQLKRRRYTGWGR | |
| | LSRKLINGIRDKQSGKTILDFLKSDGFANRNFMQLIHDDSLTFKEDI | |
| | QKAQVSGQGDSLHEHIANLAGSPAIKKGILQTVKVVDELVKVMGR | |
| | HKPENIVIEMARENQTTQKGQKNSRERMKRIEEGIKELGSQILKEH | |
| | PVENTQLQNEKLYLYYLQNGRDMYVDQELDINRLSDYDVDAIVPQ | |
| | SFLKDDSIDNKVLTRSDKNRGKSDNVPSEEVVKKMKNYWRQLLNA | |
| | KLITQRKFDNLTKAERGGLSELDKAGFIKRQLVETRQITKHVAQIL | |
| | DSRMNTKYDENDKLIREVKVITLKSKLVSDFRKDFQFYKVREINNY | |
| | HHAHDAYLNAVVGTALIKKYPKLESEFVYGDYKVYDVRKMIAKSE | |
| | QEIGKATAKYFFYSNIMNFFKTEITLANGEIRKRPLIETNGETGEIV | |
| | WDKGRDFATVRKVLSMPQVNIVKKTEVQTGGFSKESILPKRNSDK | |
| | LIARKKDWDPKKYGGFDSPTVAYSVLVVAKVEKGKSKKLKSVKE | |
| | LLGITIMERSSFEKNPIDFLEAKGYKEVKKDLIIKLPKYSLFELENG | |
| | RKRMLASAGELQKGNELALPSKYVNFLYLASHYEKLKGSPEDNEQ | |
| | KQLFVEQHKHYLDEIIEQISEFSKRVILADANLDKVLSAYNKHRDK | |
| | PIREQAENIIHLFTLTNLGAPAAFKYFDTTIDRKRYTSTKEVLDATL | |
| | IHQSITGLYETRIDLSQLGGDGGSGGSGGSGGSGGSASGSAAIAEVLL | |
| | NARCDLHAVNYHGDTPLHIAARESYHDCVLLFLSRGANPELRNKE | |
| | GDTAWDLTPERSDVWFALQLNRKLRLGVGNRAIRTEKIICRDVAR | |
| | GYENVPIPCVNGVDGEPCPEDYKYISENCETSTMNIDRNITHLQHC | |
| | TCVDDCSSSNCLCGQLSIRCWYDKDGRLLQEFNKIEPPLIFECNQA | |
| | CSCWRNCKNRVVQSGIKVRLQLYRTAKMGWGVRALQTIPQGTFI | |
| | CEYVGELISDAEADVREDDSYLFDLDNKDGEVYCIDARYYGNISRFI | |
| | NHLCDPNIIPVRVFMLHQDLRFPRIAFFSSRDIRTGEELGFDYGDRF | |
| | WDIKSKYFTCQCGSEKCKHSAEAIALEQSRLARLDPHPELLPELGS | |
| | LPPVNTSGGGSGGGSKRPAATKKAGQAKKKKGGSGSGATNFSLLKQAG | |
| | DVEENPGPAAA | |
| 93 | MPKKKRKVGGSGGSDYKDHDGDYKDHDIDYKDDDDKGGGSGGGSG | dCas9-DNMT3A: |
| | TGGSGGSGGSGGSGGSGRPMDKKYSIGLAIGTNSVGWAVITDEYKV | location effector |
| | PSKKFKVLGNTDRHSIKKNLIGALLFDSGETAEATRLKRTARRRYT | domain (denoted by |
| | RRKNRICYLQEIFSNEMAKVDDSFFHRLEESFLVEEDKKHERHPIF | bold underlined |
| | GNIVDEVAYHEKYPTIYHLRKKLVDSTDKADLRLIYLALAHMIKFR | sequence) fused to C- |
| | GHFLIEGDLNPDNSDVDKLFIQLVQTYNQLFEENPINASGVDAKAIL | terminus of dCas9. |
| | SARLSKSRRLENLIAQLPGEKKNGLFGNLIALSLGLTPNFKSNFDLA | Underlined denotes |
| | EDAKLQLSKDTYDDDLDNLLAQIGDQYADLFLAAKNLSDAILLSDI | (GGS)₅ amino acid |
| | LRVNTEITKAPLSASMIKRYDEHHQDLTLLKALVRQQKKKRKVGL | linkers (SEQ ID NO: |
| | PEKYKEIFFDQSKNGYAGYIDGGASQEEFYKFIKPILEKMDGTEEL | 75). Bold denotes |
| | LVKLNREDLLRKQRTFDNGSIPHQIHLGELHAILRRQEDFYPFLKD | dCas9. Amino acids 2- |
| | NREKIEKILTFRIPYYVGPLARGNSRFAWMTRKSEETITPWNFEEV | 9: SV40 NLS. Amino |
| | VDKGASAQSFIERMTNFDKNLPNEKVLPKHSLLYEYFTVYNELTK | acids 15-36: 3xFLAG. |
| | VKYVTEGMRKPAFLSGEQKKAIVDLLFKTNRKVTVKQLKEDYFK | Italics denote |
| | KIECFDSVEISGVEDRFNASLGTYHDLLKIIKDKDFLDNEENEDILE | Neoplasmin NLS. |
| | DIVLTLTLFEDREMIEERLKTYAHLFDDKVMKQLKRRRYTGWGR | |
| | LSRKLINGIRDKQSGKTILDFLKSDGFANRNFMQLIHDDSLTFKEDI | |
| | QKAQVSGQGDSLHEHIANLAGSPAIKKGILQTVKVVDELVKVMGR | |
| | HKPENIVIEMARENQTTQKGQKNSRERMKRIEEGIKELGSQILKEH | |
| | PVENTQLQNEKLYLYYLQNGRDMYVDQELDINRLSDYDVDAIVPQ | |
| | SFLKDDSIDNKVLTRSDKNRGKSDNVPSEEVVKKMKNYWRQLLNA | |
| | KLITQRKFDNLTKAERGGLSELDKAGFIKRQLVETRQITKHVAQIL | |
| | DSRMNTKYDENDKLIREVKVITLKSKLVSDFRKDFQFYKVREINNY | |
| | HHAHDAYLNAVVGTALIKKYPKLESEFVYGDYKVYDVRKMIAKSE | |
| | QEIGKATAKYFFYSNIMNFFKTEITLANGEIRKRPLIETNGETGEIV | |
| | WDKGRDFATVRKVLSMPQVNIVKKTEVQTGGFSKESILPKRNSDK | |
| | LIARKKDWDPKKYGGFDSPTVAYSVLVVAKVEKGKSKKLKSVKE | |
| | LLGITIMERSSFEKNPIDFLEAKGYKEVKKDLIIKLPKYSLFELENG | |
| | RKRMLASAGELQKGNELALPSKYVNFLYLASHYEKLKGSPEDNEQ | |
| | KQLFVEQHKHYLDEIIEQISEFSKRVILADANLDKVLSAYNKHRDK | |
| | PIREQAENIIHLFTLTNLGAPAAFKYFDTTIDRKRYTSTKEVLDATL | |
| | IHQSITGLYETRIDLSQLGGDGGSGGSGGSGGSGGSASRAPSRLQMF | |
| | FANNHDQEFDPPKVYPPVPAEKRKPIRVLSLFDGIATGLLVLKDLG | |
| | IQVDRYIASEVCEDSITVGMVRHQGKIMYVGDVRSVTQKHIQEWG | |
| | PFDLVIGGSPCNDLSIVNPARKGLYEGTGRLFFEFYRLLHDARPKE | |
| | GDDRPFFWLFENVVAMGVSDKRDISRFLESNPVMIDAKEVSAAHR | |
| | ARYFWGNLPGMNRPLASTVNDKLELQECLEHGRIAKFSKVRTITT | |
| | RSNSIKQGKDQHFPVFMNEKEDILWCTEMERVFGFPVHYTDVSNM | |
| | SRLARQRLLGRSWSVPVIRHLFAPLKEYFACVTSGGGSGGGSKRPAA | |
| | TKKAGQAKKKKGGSGSGATNFSLLKQAGDVEENPGPAAA | |
| 94 | MPKKKRKVGGSGGSDYKDHDGDYKDHDIDYKDDDDKGGGSGGGSG | dCas9-KRAB: location |
| | TGGSGGSGGSGGSGGSGRPMDKKYSIGLAIGTNSVGWAVITDEYKV | effector domain |
| | PSKKFKVLGNTDRHSIKKNLIGALLFDSGETAEATRLKRTARRRYT | (denoted by bold |
| | RRKNRICYLQEIFSNEMAKVDDSFFHRLEESFLVEEDKKHERHPIF | underlined sequence) |
| | GNIVDEVAYHEKYPTIYHLRKKLVDSTDKADLRLIYLALAHMIKFR | fused to C-terminus of |
| | GHFLIEGDLNPDNSDVDKLFIQLVQTYNQLFEENPINASGVDAKAIL | dCas9. Underlined |
| | SARLSKSRRLENLIAQLPGEKKNGLFGNLIALSLGLTPNFKSNFDLA | denotes (GGS)₅ amino |
| | EDAKLQLSKDTYDDDLDNLLAQIGDQYADLFLAAKNLSDAILLSDI | acid linkers (SEQ ID |
| | LRVNTEITKAPLSASMIKRYDEHHQDLTLLKALVRQQKKKRKVGL | NO: 75). Bold denotes |
| | PEKYKEIFFDQSKNGYAGYIDGGASQEEFYKFIKPILEKMDGTEEL | dCas9. Amino acids 2- |
| | LVKLNREDLLRKQRTFDNGSIPHQIHLGELHAILRRQEDFYPFLKD | 9: SV40 NLS. Amino |
| | NREKIEKILTFRIPYTVGPLARGNSRFAWMTRKSEETITPWNFEEV | acids 15-36: 3xFLAG. |
| | VDKGASAQSFIERMTNFDKNLPNEKVLPKHSLLYEYFTVYNELTK | Italics denote |
| | VKYVTEGMRKPAFLSGEQKKAIVDLLFKTNRKVTVKQLKEDYFK | Neoplasmin NLS. |
| | KIECFDSVEISGVEDRFNASLGTYHDLLKIIKDKDFLDNEENEDILE | |
| | DIVLTLTLFEDREMIEERLKTYAHLFDDKVMKQLKRRRYTGWGR | |
| | LSRKLINGIRDKQSGKTILDFLKSDGFANRNFMQLIHDDSLTFKEDI | |
| | QKAQVSGQGDSLHEHIANLAGSPAIKKGILQTVKVVDELVKVMGR | |
| | HKPENIVIEMARENQTTQKGQKNSRERMKRIEEGIKELGSQILKEH | |
| | PVENTQLQNEKLYLYYLQNGRDMYVDQELDINRLSDYDVDAIVPQ | |
| | SFLKDDSIDNKVLTRSDKNRGKSDNVPSEEVVKKMKNYWRQLLNA | |
| | KLITQRKFDNLTKAERGGLSELDKAGFIKRQLVETRQITKHVAQIL | |
| | DSRMNTKYDENDKLIREVKVITLKSKLVSDFRKDFQFYKVREINNY | |
| | HHAHDAYLNAVVGTALIKKYPKLESEFVYGDYKVYDVRKMIAKSE | |
| | QEIGKATAKYFFYSNIMNFFKTEITLANGEIRKRPLIETNGETGEIV | |
| | WDKGRDFATVRKVLSMPQVNIVKKTEVQTGGFSKESILPKRNSDK | |
| | LIARKKDWDPKKYGGFDSPTVAYSVLVVAKVEKGKSKKLKSVKE | |
| | LLGITIMERSSFEKNPIDFLEAKGYKEVKKDLIIKLPKYSLFELENG | |
| | RKRMLASAGELQKGNELALPSKYVNFLYLASHYEKLKGSPEDNEQ | |
| | KQLFVEQHKHYLDEIIEQISEFSKRVILADANLDKVLSAYNKHRDK | |
| | PIREQAENIIHLFTLTNLGAPAAFKYFDTTIDRKRYTSTKEVLDATL | |
| | IHQSITGLYETRIDLSQLGGDGGSGGSGGSGGSGGSASTLVTFKDVF | |
| | VDFTREEWKLLDTAQQIVYRNVMLENYKNLVSLGYQLTKPDVILR | |
| | LEKGEEPWLVEREIHQETHPTSGGGSGGGSKRPAATKKAGQAKKKKG | |
| | GSGSGATNFSLLKQAGDVEENPGPAAA | |

1. A fusion protein comprising (1) a catalytically inactive Cas9 (dCas9) domain and (2) an effector domain, wherein the effector domain is enhancer of zeste homolog 2 (Ezh2), Friend of GATA1 (FOG1), histone H3 lysine 9 methyltransferase G9A (G9A), histone-lysine N-methyltransferase SUV39H1 (SUV39H1), Krüppel-associated box (KRAB), or DNA (cytosine-5)-methyltransferase 3A (DNMT3A).

2. The fusion protein of claim 1, wherein the effector domain is located N-terminal and/or C-terminal to the dCas9 domain.

3. The fusion protein of claim 1, further comprising a nuclear localization signal (NLS) domain, a FLAG epitope tag, or an amino acid linker.

4. The fusion protein of claim 3, wherein the NLS domain, the FLAG epitope tag, and/or the amino acid linker are located N-terminal and/or C-terminal to the dCas9 domain.

5. The fusion protein of claim 3, wherein the amino acid linker comprises the amino acid sequence (GGS)ₙ, wherein the subscript n is the number of repeat units and is between 1 and 10 (SEQ ID NO: 95).

6. The fusion protein of claim 1, wherein the effector domain is KRAB or DNMT3A and wherein the effector domain is located N-terminal to the dCas9 domain.

7. The fusion protein of claim 1, wherein the effector domain is Ezh2 and wherein the Ezh2 effector domain comprises the conserved cysteine-rich (CXC) and Su(var)3-9, Enhancer-of-zeste and Trithorax (SET) domains.

8. The fusion protein of claim 7, wherein the Ezh2 effector domain further comprises the embryonic ectoderm development (EED) binding domain.

9. The fusion protein of claim 7, wherein the Ezh2 effector domain comprises amino acids 1-746 of Ezh2 (SEQ ID NO:1).

10. The fusion protein of claim 7, wherein the Ezh2 effector domain is located N-terminal to the dCas9 domain.

11. The fusion protein of claim 1, wherein the effector domain comprises amino acids 1-45 of FOG1 (SEQ ID NO:3), wherein a first NLS domain is located at the N-terminal end of the protein, and wherein a second NLS domain is located at the C-terminal end of the protein.

12. The fusion protein of claim 11, further comprising a FLAG epitope tag that is located between the first NLS domain and the N-terminal end of the dCas9 domain.

13. The fusion protein of claim 12, wherein the FOG1 effector domain comprises 1, 2, 3, or 4 FOG1 effector domains that are located between the FLAG epitope tag and the N-terminal end of the dCas9 domain.

14. The fusion protein of claim 13, further comprising an amino acid linker comprising the amino acid sequence (GGS)ₙ, wherein n is 5 (SEQ ID NO: 75) and wherein the amino acid linker is located between the FOG1 effector domain and the N-terminal end of the dCas9 domain.

15. The fusion protein of claim 13, further comprising an amino acid linker comprising the amino acid sequence (GGS)ₙ, wherein n is 5 (SEQ ID NO: 75) and wherein the amino acid linker is located between the C-terminal end of the dCas9 domain and the second NLS domain.

16. The fusion protein of claim 12, wherein the FOG1 effector domain is located between the second NLS domain and the C-terminal end of the dCas9 domain.

17. The fusion protein of claim 16, further comprising an amino acid linker comprising the amino acid sequence (GGS)ₙ, wherein n is 5 (SEQ ID NO: 75) and wherein the amino acid linker is located between the FLAG epitope tag and the N-terminal end of the dCas9 domain.

18. The fusion protein of claim 16, further comprising an amino acid linker comprising the amino acid sequence (GGS)ₙ, wherein n is 5 (SEQ ID NO: 75) and wherein the amino acid linker is located between the C-terminal end of the dCas9 domain and the FOG1 effector domain.

19. The fusion protein of claim 12, wherein a first FOG1 effector domain is located between the FLAG epitope tag and the N-terminal end of the dCas9 domain and a second FOG1 effector domain is located between the C-terminal end of the dCas9 domain and the second NLS domain.

20. The fusion protein of claim 19, further comprising an amino acid linker comprising the amino acid sequence (GGS)ₙ, wherein n is 5 (SEQ ID NO: 75), and wherein the amino acid linker is located between the first FOG1 effector domain and the N-terminal end of the dCas9 domain.

21. The fusion protein of claim 19, further comprising an amino acid linker comprising the amino acid sequence (GGS)ₙ, wherein n is 5 (SEQ ID NO: 75), and wherein the amino acid linker is located between the C-terminal end of the dCas9 domain and the second FOG1 effector domain.

22. A nucleic acid comprising a polynucleotide sequence encoding the fusion protein of claim 1.

23. An expression vector comprising the nucleic acid of claim 22.

24. A cell comprising the fusion protein of claim 1.

25. A method for producing an epigenetic modification of a target chromatin site comprising a Cas9 recognition site, the method comprising contacting the target chromatin site with the fusion protein of claim 1.

26. The method of claim 25, wherein the epigenetic modification comprises acetylation, deacetylation, or methylation.

27. The method of claim 26, wherein methylation comprises the addition of one, two, or three methyl groups.

28. The method of claim 25, wherein an epigenetic modification of a nucleic acid or a histone protein is produced.

29. The method of any claim 25, wherein an epigenetic modification of histone H3 is produced.

30. The method of claim 25, wherein lysine 9 on histone H3 is trimethylated (H3K9me3) and/or lysine 27 on histone H3 is trimethylated (H3K27me3).

31. The method of claim 25, wherein the epigenetic modification is produced in vitro.

32. The method of claim 25, wherein the fusion protein and the target chromatin site are in a cell.

33. The method of claim 25, further comprising contacting the target chromatin site with a single guide RNA (sgRNA).

34. The method of claim 25, wherein expression of the target chromatin site is suppressed.

35. A cell comprising the expression vector of claim 23.
